(19) United States
(12) Patent Application Publication
(10) Pub. No.: US 2019/0093147 A1
(43) Pub. Date: Mar. 28, 2019

(54) PURIFICATION PROCESS OF NASCENT DNA

(71) Applicant: CENTRE NATIONAL DE LA RECHERCHE SCIENTIFIQUE (CNRS), PARIS CEDEX (FR)

(72) Inventors: Marcel Mechali, Montferrier Sur Lez (FR); Philippe Coulombe, Montpellier (FR); Christelle Cayrou, Montpellier (FR); Eric Rivals, Montpellier (FR); Paulina Prorok, Montpellier (FR)

(21) Appl. No.: 16/155,421

(22) Filed: Oct. 9, 2018

Related U.S. Application Data
(63) Continuation-in-part of application No. 14/681,351, filed on Apr. 8, 2015, now Pat. No. 10,131,913.
Application No. 14/681,351 is a continuation-in-part of application No. 13/393,259, filed on May 2, 2012, now abandoned.
(60) Provisional application No. 61/238,315, filed on Aug. 31, 2009.

(51) Int. Cl. C12Q 1/6806; C12N 15/10; C12N 15/79

(52) U.S. Cl. CPC C12Q 1/6806; C12N 15/79; C12N 15/1003

(57) ABSTRACT

The invention relates to a method for initiating the replication of a deoxyribonucleic acid molecule, said method comprising a step of inserting, into said deoxyribonucleic acid molecule, at least one nucleic acid molecule representing a multicellular DNA replication origin, the replication origin comprising at least nine nucleotides, the at least nine nucleotides consisting of at least three uninterrupted origin repeating elements (OGRE).

This application is a continuation-in part of the patent application U.S. Ser. No. 14/681,351, filed on Apr. 8, 2015, which is a continuation-in-part of the patent application U.S. Ser. No. 13/393,259 filed on Aug. 31, 2010.

The present invention relates to a purification process of nascent DNA.

In metazoans, thousands of chromosomal sites are activated at each cell cycle to initiate DNA synthesis and permit total duplication of the genome. They all should be activated only once to avoid any amplification and maintain genome integrity. How these sites are defined remains elusive despite considerable efforts trying to unravel a possible replication origin code.

In Saccharomyces cerevisiae, DNA replication origins are specifically identified by specific DNA elements, called Autonomous Replication Sequence elements (ARS), which have a common AT-rich 11 bp specific consensus. However, sequence specificity identifies but not determines origin selection.

In multicellular organisms, it was more difficult to identify common features of DNA replication origins. No strict consensus sequence element has been found, which can have predictive value, although specific sites are recognized as DNA replication origins in chromosomes of somatic cells.

The identification of the sequence of DNA replication origin offers new perspectives in the comprehension of pathologies involving miss regulation of DNA replication, and new perspective in the cellular therapy, by using “humanized” vectors.

International application WO 98/27200 discloses a putative consensus sequence of human and mammalian replication origin. However, the consensus sequences disclosed in WO 98/27200 appears to be not representative of all the replications origins normally used in multicellular eukaryotic cells.

Prior art also discloses methods for purifying nascent DNA for mapping of DNA replication origins in multicellular eukaryotic cells [Prioleau et al. 2003, Molecular and Cellular Biology, 23(10), pages 3536-3549; Cadoret et al. 2008, P.N.A.S., 105(41), pages 15837-15842; Gomez et al. 2008, Genes & Development, 22(3), pages 375-385; Sequeira-Mendes, 2009, PloS Genetics, 5(4)].

Prioleau et al. 2003, Molecular and Cellular Biology, 23(10), pages 3536-3549 disclose methods for purification of nascent strands. The method included sucrose gradients, heat denaturation and exonuclease digestion. The heat denaturation step allows to open the DNA and separate the nascent DNA strand by sucrose gradient.

So, there is a need to provide a new consensus sequence representing all the DNA replication origins of a multicellular eukaryotic cell.

There is also a need to provide a new method for determining the DNA replication origins of a multicellular eukaryotic cell.

One aim of the invention is to provide a method for purifying RNA-primed nascent DNA in a large amount and with a very high purity.

One aim of the invention is to provide a method for identifying eukaryotic replication origin.

Another aim of the invention is to provide the sequence of said eukaryotic replication origin.

Another aim is the use of RNA-primed nascent DNA produced by said replication origin for providing a method of diagnosis.

The invention relates to the use of purified RNA-primed nascent DNA for the implementation of a process allowing the mapping and the numbering of the active DNA replication origins of multi cellular eukaryotic cells, and the characterization of the sequence of said replication origins,

said process comprising
a step of extracting a mixture of nucleic acid molecules, said mixture of nucleic acid molecules comprising DNA and RNA-primed nascent DNA, from multi cellular eukaryotic cells
a step of enrichment of RNA-primed nascent DNA from said mixture by eliminating proteins associated with said nucleic acid molecules, and
at least two steps of elimination of contaminating DNA from the mixture to recover purified nascent DNA.

The initiation of new DNA strands at origins of replication in multicellular eukaryotic cells requires de novo synthesis of RNA primers by the DNA primase activity of DNA polymerase alfa and subsequent elongation from RNA primers also by DNA polymerase alpha. These RNA-primed nascent DNA are thus hybrid molecules consisting of a short molecule of RNA fused in its 3′ end to a DNA molecule.

The inventors have demonstrated that a double treatment by lambda exonuclease in exhaustive digestion conditions allows to drastically enhances the purity of isolated nascent DNA.

The RNA-primed nascent DNAs are purified, which means that said nascent DNA are substantially pure: after one step of lambda exonuclease, contaminant DNA may represent up to 25% of the purified DNA.

According to the invention, at least two exonuclease treatments allows to eliminate contaminant DNA (after 2 steps: less than 5% of contaminating DNA is present in the mixture, after 3 steps less than 2% of contaminating DNA is present in the mixture).

In one advantageous embodiment, the invention relates to the use as defined above, wherein said RNA-primed nascent DNA are produced by the active replication origins.

In one advantageous embodiment, the invention relates to the use of purified RNA-primed nascent DNA for mapping and numbering the active DNA replication origins as defined above, wherein said process is carried out by using multicellular organism totipotent cells.

In one advantageous embodiment, the invention relates to the use of purified RNA-primed nascent DNA for mapping and numbering the active DNA replication origins as defined above, wherein process is carried out by using totipotent cells, such as ES cells or multicellular organism differentiated cells.

In one advantageous embodiment, the invention relates to the use of purified RNA-primed nascent DNA for the characterization of the sequence the active DNA replication origins as defined above, wherein said sequence consists of
the nucleic acid sequences selected from the group comprising the following sequences:

(SEQ ID NO: 1)

5′-(N₇)ₐ(N₈)_{b}(GN₁N₂)_{c}(N₇)_{d}(N₈)ₑ-3′

wherein N₁ is a G or a A and N₂ is a pyridine or a A

(SEQ ID NO: 2)

5′-(N₇)ₐ(N₈)_{b}(N₃GN₄)_{c}(N₇)_{d}(N₈)ₑ-3′

wherein N₃ is a Tor a G base and N₄ is a G or a C, and

(SEQ ID NO: 3)

5′-(N₇)ₐ(N₈)_{b}(N₅N₆G)_{c}(N₈)_{d}(N₉)ₑ-3′

wherein Nₛ is different from N₆, N₅ is a G or a C and N₆ is a Tor a A

wherein c vary from 3 to 20

wherein N₇ and N₈ represent any nucleotide,

wherein a and e independently from each other can be equal to 0, 1 2 or 3, or vary from about 15 to 30, and

wherein band d independently from each other can be equal to 0, 1 2 or 3 or vary from about 10 to 300,

N₈ being such that if b vary from 10 to 300, (N₈)_{b} represents a nucleic acid chain which is such that
it contains from about 50% to about 100% of A,
it contains from about 50% to about 100% of T,
it contains from 0% to about 10% of G, and
it contains from 0% to about 12% of C,

N₉ being such that if d vary from 10 to 300, (N₉)_{d} represents a nucleic acid chain which is such that
it contains from about 50% to about 100% of A,
it contains from about 50% to about 100% of T,
it contains from 0% to about 10% of G, and
it constrains from 0% to about 12% of C.
   or any fragment of the above sequence consisting of at least 9 nucleotides.

In the invention pyridine means T or C, or U for RNA.

In one other embodiment, the invention relates to the use of purified nascent DNA as defined above, wherein said nucleic acid sequence being such that
it contains from about 33% to about 66% of G,
it contains from about 27% to about 33% of C,
it contains from about 0% to about 12% of A,
it contains from about 0% to about 15% of T, and
it has a minimal consensus sequence chosen among the following consensus sequences:

5′-N1N2G-3′

wherein N1 is a Gor a A and N2 is a pyridine or a A

S′-N3GN4-3′

wherein N3 is a T or a G base and N4 is a G or a C, and

5′-GN5N6-3′

wherein Ns is different from N6, N5 is a G or a C and N6 is a T or a A said minimal consensus sequence being repeated from 3 to 20 times without interruption between said repeated minimal consensus sequence.

In one other embodiment, the invention relates to the use of purified RNA-primed nascent DNA. as defined above, wherein said nucleic acid sequence consists of the following sequence SEQ ID NO: 4:

5′-SHGCYGSYGGMGCYGSHGSTG-3′,

or any fragment of said nucleic acid sequence consisting of at least 9 nucleotides.

In the invention, the following nomenclature in nucleic acid sequence is used:

R represents A or G

Y represents C or T

M represents A or C

K represents G or T

S represents G or C

W represents A or T

B represents G, Tor C D represents G, A or T

H represents A, C or T

V represents G, C or A, and

N represents any nucleotide (A, T, G or C)

In one other embodiment, the invention relates to the use of purified nascent DNA as defined above, wherein said nucleic acid sequence consists of the following sequence SEQ ID NO: 5:

5′-CKGYKGCKGCDGCKGCDGYKG-3′

or any fragment of said nucleic acid sequence consisting of at least 9 nucleotides.

In one other embodiment, the invention relates to the use of purified nascent DNA as defined above, wherein said nucleic acid sequence consists of one of the following sequences

(SEQ ID NO: 6)

GTCCCAGTCCCAG

(SEQ ID NO: 7)

TGCTGCTGCTGCT

(SEQ ID NO: 8)

TATATATATATAT

(SEQ ID NO: 9)

AGCAGCAGCAGCA

(SEQ ID NO: 10)

GTTGCTGCTGCTG

(SEQ ID NO: 11)

TCAGACATCTTAG

(SEQ ID NO: 12)

AGCAGCAGCAACA

(SEQ ID NO: 13)

CAGACATCTTAGG

(SEQ ID NO: 14)

AGACATCTTAGGC

(SEQ ID NO: 15)

CAGCAGCAGCAGC

(SEQ ID NO: 16)

TAACGTGTGGTGA

(SEQ ID NO: 17)

TGTTGCTGCTGCT

(SEQ ID NO: 18)

CAGCAGCAGCAAC

(SEQ ID NO: 19)

TGCTGCTGC

(SEQ ID NO: 20)

CAGCAGCAG

(SEQ ID NO: 21)

CTGCTGCTG

(SEQ ID NO: 22)

CTCTCTCTCTCT

(SEQ ID NO: 23)

TCTCTCTCTCTC

(SEQ ID NO: 24)

AGCTGGGGCGGCA

(SEQ ID NO: 25)

CAGCTGGGGCGGC

(SEQ ID NO: 26)

GCTGGGGCGGCAG

(SEQ ID NO: 27)

AGCAGCTGGACAC

(SEQ ID NO: 28)

CAGCAGCTGGACA

(SEQ ID NO: 29)

GCAGCAGCTGGAC

(SEQ ID NO: 30)

CAGCTGGACACAC

(SEQ ID NO: 31)

AGCAGACTGGGCG.

The invention also relates to an isolated nucleic acid sequence representing multi cellular DNA replication origins, wherein said nucleic acid sequence consists of one of the following sequences
the nucleic acid sequences selected from the group comprising the following sequences:

(SEQ ID NO: 1)

5′-(N₇)ₐ(N₈)_{b}(GN₁N₂)_{c}(N₇)_{d}(N₈)ₑ-3′

wherein N₁ is a G or a A and N₂ is a pyridine or a A

(SEQ ID NO: 2)

5′-(N₇)ₐ(N₈)_{b}(N₃GN₄)_{c}(N₇)d(N₈)ₑ-3′

wherein N₃ is a Tor a G base and N₄ is a G or a C, and

(SEQ ID NO: 3)

5′-(N₇)ₐ(N₈)_{b}(N₅N₆G)_{c}(N₈)_{d}(N₉)ₑ-3′

wherein N₅ is different from N₆, N₅ is a G or a C and N₆ is a Tor a A

wherein c vary from 3 to 20

wherein N₇ and N₈ represent any nucleotide,

wherein a and e independently from each other can be equal to 0, 1, 2 or 3, or vary from about 15 to 30, and

wherein band d independently from each other can be equal to 0, 1, 2 or 3 or vary from about 10 to 300,

N₈ being such that if b vary from 10 to 300, (N₈)_{b} represents a nucleic acid chain which is such that
it contains from about 50% to about 100% of A,
it contains from about 50% to about 100% of T,
it contains from 0% to about 10% of G, and
it contains from 0% to about 12% of C,

N9 being such that if d vary from 10 to 300, (N9)d represents a nucleic acid chain which is such that
it contains from about 50% to about 100% of A,
it contains from about 50% to about 100% of T,
it contains from 0% to about 10% of G, and
it contains from 0% to about 12% of C.
or any fragment of the above sequence consisting of at least 9 nucleotides.

The invention relates to the use of an isolated nucleic acid sequence, as a multi cellular DNA replication origin wherein said nucleic acid sequence consists of
a) the nucleic acid sequence

(SEQ ID NO: 2)

5′-(N₇)ₐ(N₈)_{b}(N₃GN₄)_{c}(N₇)_{d}(N₈)ₑ-3′

wherein N₃ is a T or a G base and N₄ is a G or a C,
wherein c vary from 3 to 20
wherein N₇ and Ng represent any nucleotide,
wherein a and e independently from each other can be equal to 0, 1, 2 or 3, or vary from about 15 to 30, and
wherein b can be equal to 0, 1, 2 or 3 wherein d can be equal to 0, 1, 2 or 3 or vary from about 10 to 300,
b) or any fragment of the above sequence consisting of at least 9 nucleotides, said nucleic acid sequence being such that
   it contains from 33% to 66% of G,
   it contains from 27% to 33% of C,
   it contains from 0% to 12% of A,
   it contains from 0% to 15% of T,
   it has a minimal consensus sequence 5′-N3GN4-3′ wherein N3 is a T or a G base and N4 is a G or a C
said minimal consensus sequence being repeated from 3 to 20 times without interruption between said repeated minimal consensus sequence, for controlling the ex vivo replication of a nucleotidic sequence into a pluricellular eukaryotic cell. Advantageously, the invention relates to the use of the isolated nucleic acid sequence according the above definition, wherein said nucleic acid sequence consists of one of the following sequences:

(SEQ ID NO: 24)

AGCTGGGGCGGCA

(SEQ ID NO: 25)

CAGCTGGGGCGGC,
and

(SEQ ID NO: 26)

GCTGGGGCGGCAG.

The above sequences that correspond to DNA eukaryotic origins are novel.

The invention relates to the isolated nucleic acid sequence according to claim 10,

wherein said nucleic acid sequence being such that
it contains from about 33% to about 66% of G,
it contains from about 27% to about 33% of C,
it contains from about 0% to about 12% of A,
it contains from about 0% to about 15% of T, and
it has a minimal consensus sequence chosen among the following consensus sequences:

5′-N₁N₂G-3′

wherein N₁ is a G or a A and N₂ is a pyridine or a A

5′-N₃GN₄-3′

wherein N₃ is a T or a G base and N₄ is a G or a C, and

5′-GN₅N₆-3′

wherein N₅ is different from N₆, Ns is a G or a C and N₆ is a Tor a A said minimal consensus sequence being repeated from 3 to 20 times without interruption between said repeated minimal consensus sequence.

In one advantageous embodiment, the invention relates to the isolated nucleic acid sequence as defined above, wherein said nucleic acid sequence consists of the following sequence SEQ ID NO: 4:

5′-SHGCYGSYGGMGCYGSHGSTG-3′,

or any fragment of said nucleic acid sequence consisting of at least 9 nucleotides.

In one advantageous embodiment, the invention relates to the isolated nucleic acid sequence as defined above, wherein said nucleic acid sequence consists of the following sequence SEQ ID NO: 5:

5′-CKGYKGCKGCDGCKGCDGYKG-3′

or any fragment of said nucleic acid sequence consisting of at least 9 nucleotides.

In one advantageous embodiment, the invention relates to the isolated nucleic acid sequence as defined above, wherein said nucleic acid sequence consists of one of the following sequences

(SEQ ID NO: 6)

GTCCCAGTCCCAG

(SEQ ID NO: 7)

TGCTGCTGCTGCT

(SEQ ID NO: 8)

TATATATATATAT

(SEQ ID NO: 9)

AGCAGCAGCAGCA

(SEQ ID NO: 10)

GTTGCTGCTGCTG

(SEQ ID NO: 11)

TCAGACATCTTAG

(SEQ ID NO: 12)

AGCAGCAGCAACA

(SEQ ID NO: 13)

CAGACATCTTAGG

(SEQ ID NO: 14)

AGACATCTTAGGC

(SEQ ID NO: 15)

CAGCAGCAGCAGC

(SEQ ID NO: 16)

TAACGTGTGGTGA

(SEQ ID NO: 17)

TGTTGCTGCTGCT

(SEQ ID NO: 18)

CAGCAGCAGCAAC

(SEQ ID NO: 19)

TGCTGCTGC

(SEQ ID NO: 20)

CAGCAGCAG

(SEQ ID NO: 21)

CTGCTGCTG

(SEQ ID NO: 22)

CTCTCTCTCTCT

(SEQ ID NO: 23)

TCTCTCTCTCTC

(SEQ ID NO: 24)

AGCTGGGGCGGCA

(SEQ ID NO: 25)

CAGCTGGGGCGGC

(SEQ ID NO: 26)

GCTGGGGCGGCAG

(SEQ ID NO: 27)

AGCAGCTGGACAC

(SEQ ID NO: 28)

CAGCAGCTGGACA

(SEQ ID NO: 29)

GCAGCAGCTGGAC

(SEQ ID NO: 30)

CAGCTGGACACAC

(SEQ ID NO: 31)

AGCAGACTGGGCG

The invention also relates to a recombinant vector comprising at least one isolated nucleic acid sequence as defined above.

The above vector contains at least one origin of replication that replicates as the endogenous chromosomal DNA replication origins. Therefore, the vector is duplicated as an “endogenous chromosome”. The Inventors have shown that this replication is effective (the above origins are active).

The invention also relates to a method, for controlling the replication of a nucleotidic sequence into a pluricellular eukaryotic cell, including mammal cells, comprising the insertion of, into said nucleotidic sequence, a nucleic acid sequence as defined above.

In one advantageous embodiment, the invention relates to the method as defined above, comprising a step of introducing said nucleotidic sequence into a pluricellular eukaryotic cell.

In one advantageous embodiment, the invention relates to the method as defined above for treating pathologies involving a deregulation of DNA replication, said method comprising the administration to an individual in a need thereof of a pharmaceutically effective amount of a nucleic acid sequence as defined above.

In one advantageous embodiment, the invention relates to the use of a nucleic acid sequence as defined above, for the preparation of a drug intended for the treatment of pathologies involving a deregulation of DNA replication.

In one advantageous embodiment, the invention relates to a nucleic acid sequence as defined above, for its use for the treatment of pathologies involving a deregulation of DNA replication.

The invention also relates to a pharmaceutical composition comprising, in particular as active substance, a nucleic acid sequence as defined above, in association with a pharmaceutically acceptable carrier.

The invention also relates to a method for initiating the replication of a deoxyribonucleic acid molecule in a pluricellular eukaryotic cell or in an eukaryotic cell extract, said method comprising a step of inserting, into said deoxyribonucleic acid molecule, at least one nucleic acid molecule representing a multicellular DNA replication origin, the replication origin comprising a at least nine nucleotides sequence, the at least nine nucleotides sequence consisting of at least three uninterrupted origin repeating elements (OGRE) having the sequence N₃GN₄,

wherein N₃ is T or G and N₄ is G or C.

In the invention, “initiating the replication of a deoxyribonucleic acid molecule” means that all steps necessary for replicating a double strand DNA molecule are carried out.

Also, in the invention, the OGRE, repeated at least 3 times constitutes the core of the DNA replication origin of multicellular eukaryotic cells.

Advantageously, the invention relates to the method above-mentioned, wherein the replication origin comprises one of the following sequences:

TGCTGCTGC, TGCTGGTGC, TGCGGCTGC, TGCGGGTGC,
TGCTGCTGG, TGCTGGTGG, TGCGGCTGG, TGCGGGTGG,
TGCTGCGGC, TGCTGGGGC, TGCGGCGGC, TGCGGGGGC,
TGCTGCGGG, TGCTGGGGG, TGCGGCGGG, TGCGGGGGG,
TGGTGCTGC, TGGTGGTGC, TGGGGCTGC, TGGGGGTGC,
TGGTGCTGG, TGGTGGTGG, TGGGGCTGG, TGGGGGTGG,
TGGTGCGGC, TGGTGGGGC, TGGGGCGGC, TGGGGGGGC,
TGGTGCGGG, TGGTGGGGG, TGGGGCGGG, TGGGGGGGG,
GGCTGCTGC, GGCTGGTGC, GGCGGCTGC, GGCGGGTGC,
GGCTGCTGG, GGCTGGTGG, GGCGGCTGG, GGCGGGTGG,
GGCTGCGGC, GGCTGGGGC, GGCGGCGGC, GGCGGGGGC,
GGCTGCGGG, GGCTGGGGG, GGCGGCGGG, GGCGGGGGG,
GGGTGCTGC, GGGTGGTGC, GGGGGCTGC, GGGGGGTGC,
GGGTGCTGG, GGGTGGTGG, GGGGGCTGG, GGGGGGTGG,
GGGTGCGGC, GGGTGGGGC, GGGGGCGGC, GGGGGGGGC,
GGGTGCGGG, GGGTGGGGG, GGGGGCGGG and GGGGGGGGG.

Advantageously, the invention relates to the method above mentioned, wherein the ratio G/C in the replication origin is greater than 1.

The inventors have shown that a better efficiency is obtained when the replication origin is able to form a ternary structure that form a G-quadruplex.

In molecular biology, G-quadruplexes (also known as G-tetrads or G4-DNA) are nucleic acid sequences that are rich in guanine and are capable of forming a four-stranded structure. Four guanine bases can associate through Hoogsteen hydrogen bonding to form a square planar structure called a guanine tetrad, and two or more guanine tetrads can stack on top of each other to form a G-quadruplex. The quadruplex structure is further stabilized by the presence of a cation.

In one advantageous embodiment, the invention relates to the method according to the above definition, wherein the replication origin comprises one of the following sequences:

(SEQ ID NO: 24)

agctggggcggca,

(SEQ ID NO: 25)

cagctggggcggc,
and

(SEQ ID NO: 26)

gctggggcggcag.

In one another advantageous embodiment, the invention relates to the above-mentioned method, wherein the replication origin comprises one of the following sequences:

GGGGGCGGGGAGGGAAGGGGG (SEQ ID NO: 32), which is the replication origin of the mouse cc4 gene and

GGGGGATGGGGTTGGAATGGGGGCGGG (SEQ ID NO: 33), which is the replication origin of the mouse cc2 gene.

The invention also relates to a method for conferring autonomous replicative properties to a non self-replicating deoxyribonucleic acid molecule in a pluricellular eukaryotic cell or cell extract, said method comprising a step of inserting, into said deoxyribonucleic acid molecule, at least one nucleic acid molecule representing a multicellular DNA replication origin, the replication origin comprising at a least nine nucleotides sequence, the at least nine nucleotides sequence consisting of at least three uninterrupted origin G-rich Repeated elements (OGRE) having the sequence N₃GN₄,

wherein N₃ is T or G and N₄ is G or C.

Advantageously, the invention relates to the method above mentioned, wherein the ratio G/C in the replication origin is greater than 1.

Advantageously, the invention relates to the method above mentioned, wherein the replication origin comprises one of the following sequences:

TGCTGCTGC, TGCTGGTGC, TGCGGCTGC, TGCGGGTGC,
TGCTGCTGG, TGCTGGTGG, TGCGGCTGG, TGCGGGTGG,
TGCTGCGGC, TGCTGGGGC, TGCGGCGGC, TGCGGGGGC,
TGCTGCGGG, TGCTGGGGG, TGCGGCGGG, TGCGGGGGG,
TGGTGCTGC, TGGTGGTGC, TGGGGCTGC, TGGGGGTGC,
TGGTGCTGG, TGGTGGTGG, TGGGGCTGG, TGGGGGTGG,
TGGTGCGGC, TGGTGGGGC, TGGGGCGGC, TGGGGGGGC,
TGGTGCGGG, TGGTGGGGG, TGGGGCGGG, TGGGGGGGG,
GGCTGCTGC, GGCTGGTGC, GGCGGCTGC, GGCGGGTGC,
GGCTGCTGG, GGCTGGTGG, GGCGGCTGG, GGCGGGTGG,
GGCTGCGGC, GGCTGGGGC, GGCGGCGGC, GGCGGGGGC,
GGCTGCGGG, GGCTGGGGG, GGCGGCGGG, GGCGGGGGG,
GGGTGCTGC, GGGTGGTGC, GGGGGCTGC, GGGGGGTGC,
GGGTGCTGG, GGGTGGTGG, GGGGGCTGG, GGGGGGTGG,
GGGTGCGGC, GGGTGGGGC, GGGGGCGGC, GGGGGGGGC,
GGGTGCGGG, GGGTGGGGG, GGGGGCGGG and GGGGGGGGG.

Advantageously, the invention relates to the method above mentioned, wherein the replication origin comprises one of the following sequences:

(SEQ ID NO: 24)

agctggggcggca,

(SEQ ID NO: 25)

cagctggggcggc,
and

(SEQ ID NO: 26)

gctggggcggcag.,

wherein the replication origin comprises one of the following sequences:

(SEQ ID NO: 32)

GGGGGCGGGGAGGGAAGGGGG,
and

(SEQ ID NO: 33)

GGGGGATGGGGTTGGAATGGGGGCGGG.

The invention also relates to a process for preparing a recombinant non-naturally occurring DNA vector comprising as the unique means for replicating DNA at least one multicellular DNA replication origin, said process comprising a step of inserting into a vector at least one nucleic acid molecule representing a multicellular DNA replication origin, the replication origin comprising at a least nine nucleotides sequence, the at least nine nucleotides sequence consisting of at least three uninterrupted origin repeating elements (OGRE) having the sequence N₃GN₄,

wherein N₃ is T or G and N₄ is G or C,

wherein the replication origin is originated from a nucleic acid molecule, the nucleic acid molecule being absent in the vector before its insertion.

By “recombinant non-naturally occurring DNA vector” it is meant in the invention a vector that does not exist without man intervention.

In other words, the vectors encompassed by the invention are artificially constructed by biologists. Vectors such as artificial chromosomes (of mouse for instance) are not encompassed by the invention.

Vectors of the invention are commonly constituted by a backbone from prokaryotic or yeast vectors (such as pBR322 vector, cosmid vector, or yeast artificial chromosomes) in which has been introduced at least one replication origin according to the invention.

The invention also relates to a map referencing all the DNA replication organisms of multicellular eukaryotic cells, said map being obtainable by the process as defined above.

The invention also relates to a map referencing all the DNA replication origins of multicellular eukaryotic totipotent cells, said map being obtainable by the process as defined above.

The invention also relates to a map referencing all the DNA replication origins activated in multicellular eukaryotic differentiated cells, said map being obtainable by the process as defined above.

The invention also relates to a method for the diagnostic, preferably in vitro or ex vivo, of pathologies involving a deregulation of DNA replication in an individual, or in a biological sample from an individual, said method comprising the steps:
establishing the map referencing all DNA replication origins activated in multicellular eukaryotic differentiated cells of said individual, or of said biological sample from an individual,
comparing the map obtained in the previous step with a reference map, said reference map corresponding to the map referencing all DNA replication origins activated in multicellular eukaryotic healthy differentiated cells of said individual or of said biological sample from an individual,
concluding, from the previous comparison if said individual is afflicted by a pathology involving a deregulation of DNA replication

The invention also relates to a method for the diagnostic, preferably in vitro or ex vivo, of the genetic modification of a cell of an individual, preferably a pluripotent cell, said method comprising the steps:
establishing the map referencing all DNA replication origins activated in a cell of an individual,
comparing the map obtained in the previous step with a reference map, said reference map corresponding to the map referencing all DNA replication origins activated in an healthy cell of the same type than the cell used in the previous step,
concluding, from the previous comparison said cell have a genetic modification (genetic variation of a cell during passages in vitro).

The invention also relates to a process for purifying nascent DNA, said process comprising
a step of extracting a mixture of nucleic acid molecules, said mixture of nucleic acid molecules comprising DNA and RNA-primed nascent DNA, from multi cellular eukaryotic cells.
a step of enrichment of RNA-primed nascent DNA from said mixture by eliminating proteins associated with said nucleic acid molecules, and heat denaturation of DNA.
at least two steps of elimination of contaminating DNA from the mixture to recover purified RNA-primed nascent DNA.

In one advantageous embodiment, the invention relates to the process as defined above, for purifying RNA-primed nascent DNA allowing the localization and the numbering of the active DNA replication origins of multi cellular eukaryotic cells, said process comprising the steps:
Harvesting and lysing dividing cells to obtain DNA,
Extracting and digesting proteins anchored in DNA,
Washing DNA in ethanol,
Denaturating DNA by heating.
Purifying RNA-primed nascent DNA in a neutral 5 to 30% sucrose gradient,
Collecting fractions corresponding to DNA from about 0.5-1 kb, about 1-1.5 kb, about I 0.5-2 kb and about 2-3 kb,
Phosphorylating extremity of DNA and digesting contaminant DNA by Lambda Exoonuclease.
Repeating at least once the previous step.
Performing in parallel a measure of the background control by a similar Lamba exonuclease treatment of half of the RNA-primed nascent DNA previously isolated by sucrose gradient by a mixture of RNAse A/XRN-1 or a NaOH treatment to remove the RNA primer and treated with equal amounts of λ-Exo as samples.
Precipitating and purifying said RNA-primed nascent DNA and background control (for instance with Cyscibe-GFX kit)
Amplifying RNA-primed nascent DNA and background control (for instance WGA-Sigma kit),
Purifying amplicons, said amplicons being the products of the amplification of said RNA-primed nascent DNA obtained in the previous step,
Locating on whole genome RNA-primed nascent DNA (by DNA hybridization on WCA chip or by systematic DNA sequence of amplified DNA) to determine active replication origins, and numbering said active replication origins. The bioinformatic treatment of the data allows to measure the activity of origins above the background control.

In one advantageous embodiment, the invention relates to the process as defined above, for purifying nascent DNA allowing the localization and the numbering of all the DNA replication origins of multi cellular eukaryotic cells, said process being carried out in totipotent cells as well as on a variety of differentiating cells or cancer cells.

The invention also relates to a method for initiating the replication of a first double stranded deoxyribonucleic acid (DNA) molecule in a pluricellular eukaryotic cell, said first molecule being devoid of self-replication capabilities in a pluricellular eukaryotic cell, said method comprising
inserting into said first double stranded DNA molecule at least one multicellular DNA replication origin to obtain a second double stranded DNA molecule, said replication origin consisting essentially of a sequence from about 50 to about 800 nucleotides, said replication origin comprising at least a regulatory element (RE), and an initiation site (IS), wherein said IS is located downstream to the RE at about 50 to 800 nucleotides, preferably 250 to 350 nucleotides, the RE comprising at least a nine-nucleotide sequence consisting of at least three uninterrupted origin repeating elements (OGRE), each OGRE having one of the following sequences:
   N₁N₂G, wherein N₁ is a G or a A and N₂ is a pyrimidine or a A;
   N₃GN₄, wherein N₃ is T or G and N₄ is G or C; and
   GN₅N₆, wherein N₅ is different from N₆, N₅ is a G or a C and N₆ is a T or a A;
introducing said second double stranded DNA molecule into a pluricellular eukaryotic cell; and then
identifying the replicated molecules and measuring their efficiency of replication,
wherein the replication of said second DNA molecule comprising said DNA replication origin is solely initiated, in a pluricellular eukaryotic cell, by the presence of said replication origin.

Advantageously, the step of identifying replicated molecules above mentioned is carried out by identifying the nascent DNA synthesized from the IS of the inserted DNA replication origin in said a second double stranded DNA molecule, the nascent DNA identifying said IS.

According to the method disclosed above, it is possible to induce the replication of a double stranded DNA molecule (dsDNA) devoid of replicative properties, by inserting a replication origin characterized by the inventors.

The replication origin identified by the inventors contains is a sequence of about 50 nucleotides to about 800 nucleotides, containing two specific domains involved in the replication of DNA:
A regulatory element RE which is a G-rich sequence,
An initiation site IS, where the loop which is necessary to initiate the replication of the dsDNA opens.

According to the process largely discussed in the invention it is easy to determine an IS, since this site can be identified by the nascent DNA, i.e. the newly synthetized DNA by the DNA polymerase during the progression of the replication loop.

The inventors also identify that the IS is located downstream to a regulatory region having a large number of G nucleotides (G; called “G-rich region”) which is characterized by the fact that it contains at least 3 uninterrupted repeats of a core element (OGRE) each OGRE having one of the following sequences:
N₁N₂G, wherein N₁ is a G or a A and N₂ is a pyrimidine or a A;
N₃GN₄, wherein N₃ is T or G and N₄ is G or C; and
GN₅N₆, wherein N₅ is different from N₆, N₅ is a G or a C and N₆ is a T or a A.

Unfortunately, and contrary to lower eukaryotes and bacteria, the replication origins of the pluricellular eukaryotes, including mice and humans, are not defined by a unique strict sequence, but by specific domains having a relative flexibility of sequence to encompass the complex structure of chromosomes and the extreme variability of situations where DNA has to be replicated.

This is why, before the invention, the skilled person was not able to characterize such replications origins.

Moreover, advantageously, the inventors identify that the RE is may be able to form G-quadruplex structures.

The inherent propensity of G to self-associate forming four-stranded helical structures has been known since the early 1960s (1). Subsequently it was demonstrated that the conserved DNA sequence repeats of telomeres form G-quadruplex (or G4) structures in vitro. Since then numerous biochemical and structural analyses have established that nucleic acid sequences, both DNA and RNA, containing runs of guanines (G-tracts) separated by other bases spontaneously fold into G-quadruplex structures in vitro. The building blocks of G-quadruplexes are G-quartets that are formed through a cyclic Hoogsten hydrogen-bonding arrangement of four guanines with each other. The planar G-quartets stack on top of one another forming four-stranded helical structures. G-quadruplex formation is driven by monovalent cations such as Na+ and K+, and hence physiological buffer conditions favor their formation. G-quadruplex structures are topologically very polymorphic and can arise from the intra- or inter-molecular folding of G-rich strands. Intra-molecular folding requires the presence of four or more G-tracts in one strand, whereas inter-molecular folding can arise from two or four strands giving rise to parallel or antiparallel structures depending on the orientation of the strands in a G-quadruplex.

In an advantageous embodiment, the invention relates to the method as defined above, wherein the replication origin comprises one of the following sequences:

(SEQ ID NO: 34)

TGGGAGTGGGTGGGTAGGGGAGCAGGGCGGGT;

(SEQ ID NO: 35)

CGGGGAGGGGACGAAGGGGAGAGAGGATGGGGA;

(SEQ ID NO: 36)

TGGTGGGGGTGGGGGTAGGGGTGGGGGGAGA;

(SEQ ID NO: 37)

AGGGGGAGTGGGTGGGTAGGGGATTGGGGGGGT;

(SEQ ID NO: 38)

AGGAGGGGGATGTGGAGGAGGAGGGGGAGGGGGGGT;

(SEQ ID NO: 39)

TGGGCTGGGGGCTGGAGGGGGGGATACTGGGA;

(SEQ ID NO: 40)

AGGGGACAGGGGCAGGGGTGGT;

(SEQ ID NO: 41)

AGGGGGAGGAGAGAGGGGGTAGGGGA;

(SEQ ID NO: 42)

TGGGAAGGGTTTTTTTGGGGGGGCGGGA;

(SEQ ID NO: 43)

TGACTTTGGGGGTGGGGTGGGGT;

(SEQ ID NO: 44)

AGGGTTGGGGGAGGGGGATGGAGA;

(SEQ ID NO: 45)

TGGTGGGGAGGGTGGTGGGGTGGGGA;

(SEQ ID NO: 46)

AGGGGGATGGGAGGGGGAATGGGGGC;

(SEQ ID NO: 47)

AGGGGGAGTGGGAGTGTAGGGGATCGGGGGGGGGGGTGTGAGTATGGGG
GA;

(SEQ ID NO: 48)

AGGGAGAGGGAGGATGGGATAGGGGGT;

(SEQ ID NO: 49)

TGGTAGGGGTGGGGTGGGGGCTGGGGC;

(SEQ ID NO: 50)

TGGGGATGGGGTTGGGGGTAGGGGGA;

(SEQ ID NO: 51)

AGGGCAGGCGGGGGTGGGGGGGTGGGGGTGGGGGC;

(SEQ ID NO: 52)

AGGGTGGGTGGGTGGGTGGGTGGGTGGGGC;

(SEQ ID NO: 53)

TGGCGGGGCGGGGGGGGGGTGCGGGGGAGGT;

(SEQ ID NO: 54)

AGGAGGGAGGGAGGGAGGGAGGGA;

(SEQ ID NO: 55)

TGGGGGTGGGTGGGTATGGGGGA;

(SEQ ID NO: 56)

CGGGGGGGATGGGGGGTCCTCAGGGGGCAGGGA;

(SEQ ID NO: 57)

CGCTGTGGGGTGGGGTGGGGTGGGGGAGA;

(SEQ ID NO: 58)

TGGCATGGGGGTCCGGGGGAGGGT;

(SEQ ID NO: 59)

GG[CG]G[CG]GGG[CG]G[CG]GG[CG]GGGGG[CG]GG[CG]G[CG];

(SEQ ID NO: 60)

GG[CG][CG]GGGG[CG][CG]GGGG[ACG]GGGGG[CG]GGGGGG
[AG]G[CG];

(SEQ ID NO: 61)

GGG[GT]GGGGG[CG]GGGGGG[AG]GGGGGGGGGG[CG];

(SEQ ID NO: 62)

GG[AG]GGGGGG[AC]GGGGCGGGGG[CG][CG]GGGGC[GT]GGG CC;

(SEQ ID NO: 63)

GGG[CG]GGG[CG]G[CG]GGGGCGGGG[CG][CG] CC;

(SEQ ID NO: 64)

GGG[AG]GG[CG]GGCGGGGCGGGG[CG][CG];

(SEQ ID NO: 65)

[CG]G[CG][GT]GGGGG[CG]GGGGGG[CG]GGGGG[CGT]GGGG
[CG][CG]GGGG[CG]GGGG;

(SEQ ID NO: 66)

GG[CG]G[AG]GGGGG[CG]GGGGGG[AG]GGGG[CG];

(SEQ ID NO: 67)

G[CG][GT][CG]GGGG[CG][CG]GGGGG[CG]GGGG[CG]GGGGGG
[AG]GGGGG[CG]GG;

(SEQ ID NO: 68)

GGGGGAGGGG[AG]GGG CC;

(SEQ ID NO: 69)

GGG[ag]GGG[AG]GGG[ag]GGG[ag]GGG[ag]GGG[ag]GGG
[ag]GGG[AG]GgG[ag]GGG[ag]GGG;

(SEQ ID NO: 70)

GGGGGGGGGGG[AG]GGGGGGG[AG]GGGGGGG[AG];

(SEQ ID NO: 71)

gGGGG[ag]G[ag]GGGG[ag]GGGg[AG][gt]GGGGGG[ag]
[gt][ag][gt] CC;

(SEQ ID NO: 72)

G[AG]GGG[AG]G[AG]GGGA[AG]GGGG[AG]G[AGT]GGG[AG]
GGG[AG]GGG[AG]GG;

(SEQ ID NO: 73)

G[AG]GGG[AG]GGG[AG]GGG[AG]GGG[AG]GG[AG][AG]
GGG[AG]GGG;

(SEQ ID NO: 74)

[ag]GGG[ag]gGGGGGGaGGG[ag]G[gt]GGGGG
[ag] CC;

(SEQ ID NO: 75)

GGg[AG]GG[ag][ag]GGGGGG[ag]GGGG[ag]gGg CC;

(SEQ ID NO: 76)

GGG[AG]GGGGGGGAGGGGG[GT]GGGGG[AG];

(SEQ ID NO: 77)

GG[GT]GGG[GT][AG]GGGG[AG]GGGGG[GT]GGGGGG
[AG][GT][AG][GT];
and

(SEQ ID NO: 78)

GG[GT]GGGG[GT]GGGGGG[GT]GG;

wherein for instance [AG] represents A or G, and where a square bracket represents only one nucleotide.

Advantageously, the invention relates to the method mentioned above, wherein said RE controls progression of a replication loop initiated in said IS.

The inventors identify that the RE region may control the activity and progression of the initiation loop, i.e. the loop which occurs when the two stand of a dsDNA opens and which allows replication of DNA by a DNA polymerase.

The invention also relates to a process for preparing a recombinant non-naturally occurring double stranded DNA multicellular eukaryotic replicative vector, or replicative vector, comprising at least one multicellular DNA replication origin as the unique means for replicating the vector in a pluricellular eukaryotic cell or cell extract, said process comprising a step of

inserting into a first recombinant non-naturally occurring double stranded DNA vector at least one DNA molecule comprising at least one multicellular DNA replication origin in order to obtain a multicellular eukaryotic replicative vector, said replication origin consisting essentially of a sequence from about 50 to about 800 nucleotides, said replication origin comprising at least a regulatory element (RE), and an initiation site (IS), wherein said IS is located downstream to the RE at about 50 to 800 nucleotides, the RE comprising at least a nine-nucleotide sequence consisting of at least three uninterrupted origin repeating elements (OGRE), each OGRE having one of the following sequences:

N1N2G, wherein N1 is a G or a A and N2 is a pyrimidine or a A;

N3GN4, wherein N3 is T or G and N4 is G or C; and

GN5N6, wherein N5 is different from N6, N5 is a G or a C and N6 is a T or a A;

introducing said replicative vector into a pluricellular eukaryotic cell; and then recovering the vectors resulting from the replication;

wherein said a first recombinant non-naturally occurring double stranded DNA vector being devoid of self-replicative capabilities in a pluricellular eukaryotic cell, and

wherein the inserted at least one multicellular DNA replication origin allows said DNA vector to self-replicate in a pluricellular eukaryotic cell or cell extract.

Advantageously, the invention relates to the method as defined above, wherein the at least one multicellular DNA replication origin comprises at least one of the sequences as set forth in SEQ ID NO: 39 to SEQ ID NO: 81.

Advantageously, the invention relates to the method as defined above, wherein said RE forms a potential G quadruplex structure.

Advantageously, the invention relates to the method as defined above, wherein said RE interacts with a preRC complex.

The invention also relates to a double stranded DNA vector comprising as its unique replicative DNA replication origin a replication origin consisting essentially of a sequence from about 50 to about 800 nucleotides, said replication origin comprising at least a regulatory element (RE), and an initiation site (IS), wherein said IS is located downstream to the RE at about 50 to 800 nucleotides, the RE comprising at least a nine-nucleotide sequence consisting of at least three uninterrupted origin repeating elements (OGRE), each OGRE having one of the following sequences:

N1N2G, wherein N1 is a G or a A and N2 is a pyrimidine or a A;

N3GN4, wherein N3 is T or G and N4 is G or C; and

GN5N6, wherein N5 is different from N6, N5 is a G or a C and N6 is a T or a A;

wherein said vector is devoid of bacterial or unicellular eukaryotic replication origin.

Advantageously, the invention relates to the vector as defined above, wherein the at least one multicellular DNA replication origin comprises at least one of the sequences as set forth in SEQ ID NO: 39 to SEQ ID NO: 81.

Advantageously, the invention relates to the vector as defined above, said RE forms a potential G quadruplex structure.

Advantageously, the invention relates to the vector as defined above, wherein said RE interacts with the preRC complex.

### LEGEND TO THE FIGURES

FIGS. 1 A-I represent the association between genes and replication origins

FIG. 1A corresponds to a schematic representation of a gene, in which Tss (transcription initiation site), exon and intron are represented.

FIG. 1 B represents an example of the distribution of replication origins found on a 200 kb region of MEF cells and ES cells. Negative controls for the mouse cells are the P19 asynchronous cells or P19 arrested in late mitosis by nocodazole.

FIG. 1C represents an example of the distribution of replication origins found on a 200 kb region of Kc cells. Negative control for Kc cells comes from fragmented total DNA of mitotic cells and then treated by lambda exonuclease.

FIG. 1D represents a pie chart showing the percentage of origin sequences in genes sequences (light grey) and intergenic sequences (dark grey) in MEF cells. The value of gene association for randomized origins is indicated by the dashed pie (53%). Similar values were obtained for ES and P19 cells. (*:p<0.001)

FIG. 1E represents a graph showing the percentage of origin sequences in promotor sequences (white) and intronic sequences (light grey) and exonic sequences (dark grey) in MEF cells, ES cells and P19 cells. (*:p<0.001)

FIG. 1F represents a pie chart showing the percentage of origin sequences in genes sequences (light grey) and intergenic sequences (dark grey) in drosophila Kc cells. The value of gene association for randomized origins is indicated by the dashed pie (62%). (* :p<0.001)

FIG. 1G represents a graph showing the percentage of origin sequences in promotor sequences and intronic sequences and exonic sequences in drosophila Kc cells. The value of association for randomized origins is indicated by the dashed boxes. (* :p<0.001).

FIG. 1H represents a graph showing the association of replication origins with highly transcribed genes in MEF cells. The transcriptional output of gene associated (+) or not (−) with replication origins is indicated. The average transcription of genes associated with randomly distributed origins is also shown. (*:p<0.001)

FIG. 1I represents a graph showing the association of replication origins with highly transcribed genes in drosophila Kc cells. The transcriptional output of gene associated (+) or not (−) with replication origins is indicated. The average transcription of genes associated with randomly distributed origins is also shown. (*:p<0.001).

FIGS. 2 A-K represent the association between CpG Islands and replication origins

FIG. 2A represents the sum of all the RNA-primed nascent DNA signals (corresponding to replication origins) around the site of initiation of transcription (TSS: Transcription Start Sites) in mouse MEF. Shown is the cumulative Nascent strand signal associated with all TSS (black line) and TSS associated with active replication origins (gray line).

FIG. 2B represents the sum of all the Nascent Strands signals around TSS associated with CpG Islands (CGI, light grey line) or not associated (dark grey line) in mouse MEF.

FIG. 2C represents an example of the association of replication origins of MEF, ES and P19 cells with CpG Islands. Shown is the localization of genes, CpG islands and Nascent Strands signals.

FIG. 2D represents Venn diagram showing the strong association between replication origins and CpG Islands in mouse MEF. The percentage of association is indicated.

FIG. 2E represents the sum of all the Nascent Strands signals (corresponding to replication origins) around the site of initiation of transcription (TSS: Transcription Start Site) in drosophila Kc cells. Shown is the cumulative Nascent strand signal associated with all TSS (line ‘b’) and of TSS associated with active replication origins (line ‘a’) in proliferating cells. The cumulative signal of all TSS of mitotic and non-proliferating Kc cells is also shown (line ‘c’).

FIG. 2F represents an example of the association of replication origins of drosophila Kc cells with CpG Islands-like sequences. Shown is the localization of genes, CpG islands and Nascent Strands signals in proliferating and mitotic cells.

FIG. 2G represents Venn diagram showing the strong association between replication origins and CpG Islands in drosophila Kc cells. The percentage of association is indicated.

FIG. 2H represents the sum of all the Nascent Strands signals (corresponding to replication origins) around the CpG Islands in mouse MEF. Shown is the cumulative Nascent strand signal of all CpG Islands (grey line) and CpG Islands associated with active replication origins (black line).

FIG. 2I represents the size of replication origins with regard to their association with CpG islands. The lines show the frequency of finding a replication origin of a particular length. All origins (black line) and origins associated (light grey) with CpG islands or not (dark grey line) in MEF are illustrated.

FIG. 2J represents the sum of all the Nascent Strands signals (corresponding to replication origins) around the CpG Islands-like sequences in mouse MEF. Shown is the cumulative Nascent strand signal of all CpG Islands (line ‘b’) and of CpG Islands associated with active replication origins (line ‘a’) in proliferating cells. The cumulative signal of all CpG Islands of mitotic and non-proliferating Kc cells is also shown (line ‘c’).

FIG. 2K represents the size of replication origins with regard to their association with CpG islands. The lines show the frequency of finding a replication origin of a particular length. All origins (‘square’ line) and origins associated (‘diamond’ line) with CpG islands or not (‘triangle’ line) in Kc cells are illustrated.

FIGS. 3A-3G represent the common conserved motif in Metazoan replication origins.

FIG. 3A illustrates the consensus element found in metazoan replication origins. The ‘OGRE’ (for Origin G-rich Repeated Element) motif was generated using MEME server with drosophila origins. Also shown is a randomized motif to evaluate the specificity of the OGRE. The size of letter represents the base preference for every position of the motif.

FIG. 3B represents Venn diagram showing the strong association between replication origins and occurrences of the OGRE in drosophila cells. The much weaker overlap between origins and the randomized motif is shown. The percentage of association is indicated.

FIG. 3C represents an example of the association of replication origins of Kc cells with occurrences of the OGRE. Shown is the localization of genes, CpG islands-like sequences, Nascent Strands signals and occurrences of OGRE and randomized OGRE.

FIG. 3D represents the sum of all the Nascent Strands signals (corresponding to replication origins) around occurrences of the OGRE in drosophila Kc cells. Shown is the cumulative Nascent strand signal associated with non-orientated motif (grey shadow) or with oriented OGRE (black line). The x-axis represents the distance (in base pair) from OGRE occurrences. The y-axis corresponds to cumulative p-value.

FIG. 3E represents an example of the association of replication origins of P19 cells with occurrences of the OGRE. Shown is the localization of genes, CpG islands, Nascent Strands signals and occurrences of OGRE and randomized OGRE.

FIG. 3F represents Venn diagram showing the strong association between replication origins and occurrences of the OGRE in mouse MEP cells. The much weaker overlap between origins and the randomized motif is shown. The percentage of association is indicated.

FIG. 3G represents the sum of all the Nascent Strands signals (corresponding to replication origins) around occurrences of the OGRE in drosophila P19 cells. Shown is the cumulative Nascent strand signal associated with non-orientated motif (grey shadow) or with oriented OGRE (black line). The x-axis represents the distance (in base pair) from OGRE occurrences. The y-axis corresponds to cumulative p-value.

FIGS. 4A-L represent the grouping into functional clusters along the chromosome of Metazoan replication origins.

FIG. 4A shows an example of single-molecule analysis of the inter-origin spacing by molecular combing of DNA in Kc cells by two pulse labeling. The inferred position of replication origins is shown.

FIG. 4B illustrates the distribution of the inter-origin distances in Kc cells. The x-axis represents the inter-origin spacing in kb while the frequency in shown on the y-axis.

FIG. 4C shows an example of single-molecule analysis of the inter-origin spacing by molecular combing of DNA in MEF cells by two pulse labeling. Very similar results were obtained for ES cells.

FIG. 4D illustrates the distribution of the inter-origin distances in MEF cells. The x-axis represents the inter-origin spacing in kb while the frequency in shown on the y-axis. Very similar results were obtained for ES cells.

FIG. 4E illustrates the distribution of the inter-origin distances obtained from combing data (grey bars) and from micro-array analysis (blue bars) in Kc cells. The x-axis represents the inter-origin spacing in kb while the frequency in shown on the y-axis.

FIG. 4F illustrates the distribution of the inter-origin distances obtained from combing data (grey bars) and from micro-array analysis (blue bars) in MEF cells. The x-axis represents the inter-origin spacing in kb while the frequency in shown on the y-axis. Very similar results were obtained for ES cells.

FIG. 4G illustrates the Purely Stochastic Model of Ori firing. In this model, Oris are completely independent and are activated randomly (red cercles). Very short and long inter-origin distances are observed.

FIG. 4H illustrates the Hierarchical Stochastic Model. In this model, Oris are linked within functional units where activation of one Ori silences the others in the same group.

FIG. 4I shows the distribution of the inter-origin distances obtained from combing data of Kc cells (light grey bars) and from computational simulations (dark grey bars). In the tested model, replication origins were picked at random. Note the presence of short (arrow) and long (arrowhead) inter-origin distances in the simulated dataset not found in the combing analysis. The x-axis represents the inter-origin spacing in kb while the frequency in shown on the y-axis.

FIG. 4J shows the distribution of the inter-origin distances obtained from combing data of MEF cells (light grey bars) and from computational simulations (dark grey bars). In the tested model, replication origins were picked at random. Note the presence of short (arrow) and long (arrowhead) inter-origin distances in the simulated dataset not found in the combing analysis. The x-axis represents the inter-origin spacing in kb while the frequency in shown on the y-axis. Very similar results were obtained for ES cells.

FIG. 4K shows the distribution of the inter-origin distances obtained from combing data of Kc cells (light grey bars) and from computational simulations (light grey bars). In the tested model, replication origins are clustered into functional groups where the firing of one randomly chosen replication origin suppresses the activation of the other origins within the same group. Both set of data correlate well. The x-axis represents the inter¬origin spacing in kb while the frequency in shown on the y-axis.

FIG. 4L shows the distribution of the inter-origin distances obtained from combing data of MEF cells (light grey bars) and from computational simulations (light grey bars). In the tested model, replication origins are clustered into functional groups where the firing of one randomly chosen replication origin suppresses the activation of the other origins within the same group. Both set of data correlate well. The x-axis represents the inter¬origin spacing in kb while the frequency in shown on the y-axis. Very similar results were obtained for ES cells.

FIGS. 5 A-D represent the domains of origin density correlated with domains of CpG island density and replication timing

FIG. 5A represents the totality of the 60 MB on the region defined for the mouse chromosome 11. Diagrams show the replication timing, CpG island density, exon and gene density and replication origins density for mouse cells. The panels below represent the significant overlay of MEF origins and CpG or replication timing domains. The region analyzed in Figure SB and SC are highlighted.

FIG. 5B represents a 3.S Mb region of mouse chromosome 11. Note that all indicators are relatively high in this early replication region as defined in ES cells.

FIG. 5C represents a 3.5 Mb region of mouse chromosome 11. Note the differences in origin density between MEF and pluripotent cells in the late replicating domain.

FIG. 5D shows a model illustrating genomic distribution and usage of replication origins in metazoan. Multiple loops could cluster several fired replication origins in foci. For illustration purposes, BrdU positive replication foci are shown (top panel). CpG Island could be a regulatory element for location and for efficiency firing of replication origins. In this model one origin by cluster can be fired in each cell.

FIGS. 6 A-D represent the purification process of Nascent Strands DNA from cultured cells.

FIG. 6A shows the scheme used for the purification and the analysis of metazoan replication origins.

FIG. 6B shows the analysis of the fraction obtained after the sucrose ultracentrifugation step. Fractions were analyzed by alkaline agarose gel electrophoresis. In this particular experiment, proteinase K (PK) was added (+) or not (−) during lysis. Fractions of 0.5-2 kb DNA are pooled (black box) for the following step.

FIG. 6C illustrates the specificity of lambda exonuclase. DNA (upper panel) or RNA (lower panel) samples were incubated with (+) or without (−) lambda exonuclease. The reaction was separated by agarose gel electrophoresis and visualized using GelRed staining.

FIG. 6D shows that Nascent Strands signals from microarrays can be observed by qPCR in mouse P19 and ES cells. Genes localization, Nascent Strands signals and qPCR analysis are shown.

FIGS. 7 A-C represent the reproducibility of Nascent Strands purification.

FIG. 7 A show scatter plots comparing two biological replicates of purified Nascent Strands from P19 cells. Every dot represents a single probe on the microarray. Its position is determined by the value of the log ratio of the two compared replicates. The coefficient of determination (R2) is 0.7935912.

FIG. 7B show scatter plots comparing two biological replicates of purified Nascent Strands from Kc cells. Every dot represents a single probe on the microarray. Its position is determined by the value of the log ratio of the two compared replicates. The coefficient of determination (R2) is 0.7057634.

FIG. 7C show scatter plots comparing two biological replicates of purified Nascent Strands from ES cells. Every dot represents a single probe on the microarray. Its position is determined by the value of the log ratio of the two compared replicates. The coefficient of determination (R2) is 0.3724884.

FIGS. 8 A-F represent the confirmation using qPCR analysis of replication origins identified by microarrays.

FIG. 8A represents replication origins analysis of the LoxB locus. Shown is the localization of genes, the Nascent Strands signals from microarray analysis and qPCR analysis for ES and P 19 cells.

FIG. 8B shows that our Nascent Strands preparation contains a known origin.

Represented is a qPCR analysis of the replication origin of e-mye gene.

FIGS. 8C-8F show that novel replication origins identified in our microarrays can be observed by qPCR in mouse P19 and ES cells. Genes localization, Nascent Strands signals and qPCR analysis are shown. In Figure SC, SD and 8F, the upper panel of microarray data is for ES cells while the lower panel is for P19 cells. In Figure SE, results for ES cells are shown.

FIGS. 9A-F represent the cell cycle distribution of cells used for the Nascent Strands purifications.

The DNA content of individual cells is stained and quantified using a flow cytometer. The populations of cells before (2n) and after (4n) DNA replication are indicated. Cells in between 2n and 4n are replicating DNA.

FIG. 9A represents DNA content of MEF cells actively proliferating FIG. 9B represents DNA content of ES cells actively proliferating. FIG. 9C represents DNA content of P19 cells actively proliferating. FIG. 9D represents DNA content of P19 cells arrested in mitosis. FIG. 9E represents DNA content of Kc cells actively proliferating.

FIG. 9F represents DNA content of Kc cells arrested in mitosis.

FIGS. 10 A-H represent the association between CpG Islands and replication origins in ES and P19 cells.

FIG. 10A represents the sum of all the Nascent Strands signals (corresponding to replication origins) around the site of initiation of transcription (TSS: Transcription Start Sites) in mouse ES cells. Shown is the cumulative Nascent Strands signals associated with all TSS.

FIG. 10B represents the sum of all the Nascent Strands signals around TSS associated with CpG Islands (CGI, light grey line) or not associated (dark grey line) in mouse ES cells.

FIG. 10C represents the sum of all the Nascent Strands signals (corresponding to replication origins) around the CpG Islands in mouse ES cells. Shown are the cumulative Nascent Strands signals of all CpG Islands.

FIG. 10D represents Venn diagram showing the strong association between replication origins and CpG Islands in mouse ES cells. The percentage of association is indicated.

FIG. 10E represents the sum of all the Nascent Strands signals (corresponding to replication origins) around the site of all initiation of transcription (TSS: Transcription Start Sites) in mouse P19 cells.

FIG. 10F represents the sum of all the Nascent Strands signals around TSS associated with CpG Islands (CGI, light grey line) or not associated (dark grey line) in mouse P19 cells.

FIG. 10G represents the sum of all the Nascent Strands signals (corresponding to replication origins) around the CpG Islands in mouse P 19 cells. Shown are the cumulative Nascent Strands signals of all CpG Islands.

FIG. 10H represents Venn diagram showing the strong association between replication origins and CpG Islands in mouse P 19 cells. The percentage of association is indicated.

FIG. 11 A-B correspond to a schematic representation of the Replication origin mapping by RNA-primed nascent DNA enrichment assay.

FIG. 11A is a schematic representation of the process: Nascent strands are purified and then analyzed by qPCR. Brocken lines represent nascent DNA, black boxes represent RNA primers.

FIG. 11B represents the detailed process. Cells are first lysed in the DNAzol then purified and total DNA is heated and placed on a sucrose gradient. The sucrose fractions containing DNA fragments of interest between 500 and 2000 base pairs are once phosphorylated by T4 polynucleotide kinase and then digested by lambda exonuclease. After extraction by phenol-chloroform, DNA remaining was again treated with T4 PNK and lambda exonuclease. Purified nascent strands are analyzed by qPCR. Grey lines represent contaminant DNA.

FIGS. 12 A-F represent the improvement of purification steps of nascent strains FIG. 12A represents the migration in an agarose gel of nascent strands recovered at the end of the purification after sucrose fractionation, after treatment (+PK) or not (−PK) of cell lysate obtained with DNAzol, with T4 PNK kinase.

FIG. 12B represents an histogram showing the increase of the amount and enrichment of nascent strands on hoxB9 locus. NS means Nascent strands. Black columns correspond to DNA treated with T4 PNK, and grey columns correspond to non-treated DNA.

FIG. 12D represents Hoxb9 locus. Black boxes represent genes and triangles represent primers used for qPCR. Scale: in kilobases FIG. 12D represents an histogram showing the increase of enrichment after second round of T4 PNK+lambda exonuclease treatment on hoxb9 origin. Y-axis corresponds to enrichment.

FIG. 12E represents an histogram showing the increase of enrichment, of nascent strands of 1-1. Skb after second round of T4 PNK+lambda exonuclease treatment on hoxb9 origin. Y-axis corresponds to enrichment. NS means nascent strand.

FIG. 12F represents an histogram showing the increase of enrichment, of nascent strands of 1-1.Skb after second round of T4 PNK+lambda exonuclease treatment on c-myc origin. Y-axis corresponds to enrichment. NS means nascent strand.

FIGS. 13A and B illustrate the efficiency of the replication origin on plasmids

FIG. 13A represents the procedure followed for the experiment.

FIG. 13B represents a graph showing the plasmid enrichment (i.e the DNA replication) compared to c-myc origin. A: OriP, a replication origin of a virus, B: c-myc origin, C: WT OGRE, D: Delta OGRE and E: Modified OGRE.

FIGS. 14 A to B show that deletions of the G4 element in the OGRE motifs strongly affects DNA replication origin activity Two known origins (CC2 and CC4) were selected, which contain OGRE/G4 elements. Using the CRISPR/cas method, G4 elements were deleted and the replication origin activity analysed at the corresponding loci as well as in a known control region containing (Myc 2) or not containing (Myc 12) an origin. The analysis of replication origin activity was performed by the purification of nascent strands, followed by qPCR analysis.

FIG. 14A represents the results in cc2 gene and compares WT cc2 origin to an heterozygote deletion of the cc2 origin.

FIG. 14B represents the results in cc4 gene and compares WT cc2 origin to an homozygote deletion of the cc4 origin.

FIG. 15A. DNA replication origin mapping by nascent DNA strand isolation.

Short nascent DNA strands (0.5-2 kb) were isolated by purification and denaturation of the

genomic DNA and isolation of nascent strands on sucrose gradients. The nascent strand population was further treated by exhaustive λ-exonuclease digestion, as described in Cayrou et al (2015) and Methods. The background level which might be left after the λ-exonuclease digestion was measured by treating half of the sample containing the nascent DNA strands with RNAseA/RNaseT2 prior to another λ-exonuclease digestion. Purified nascent strands were then analysed by qPCR or high-throughput whole-genome sequencing.

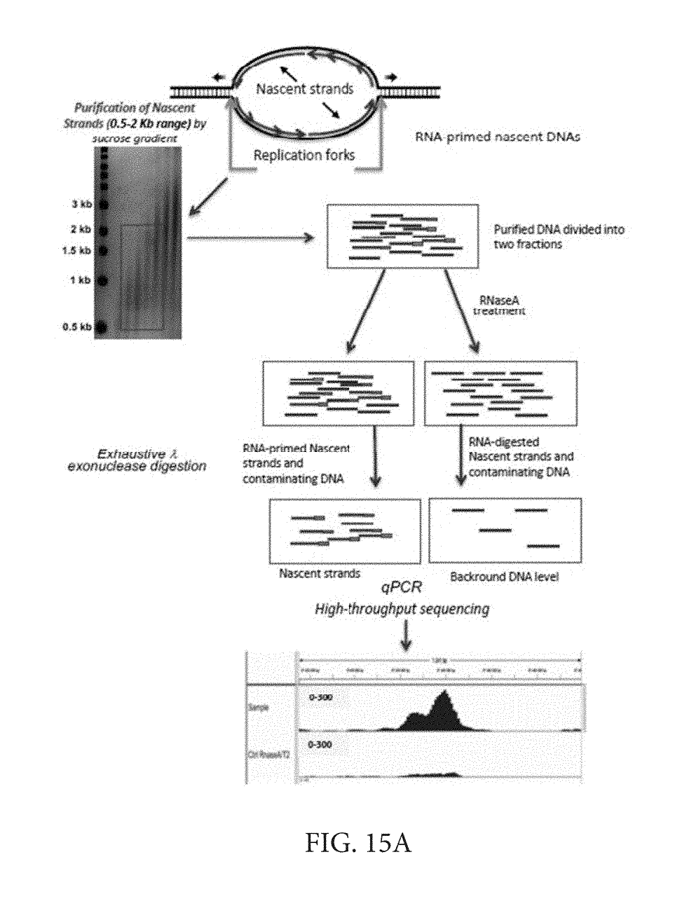

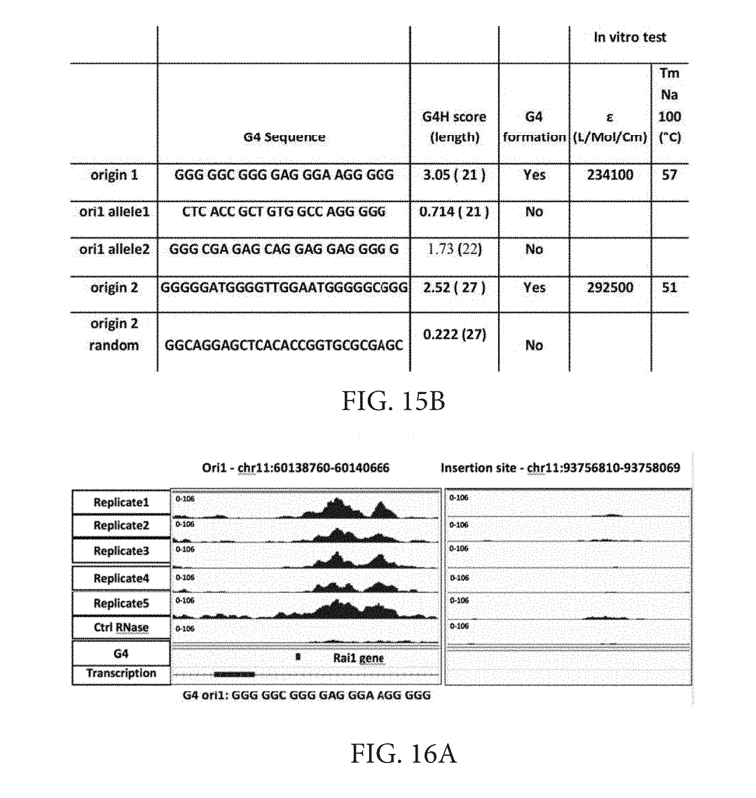

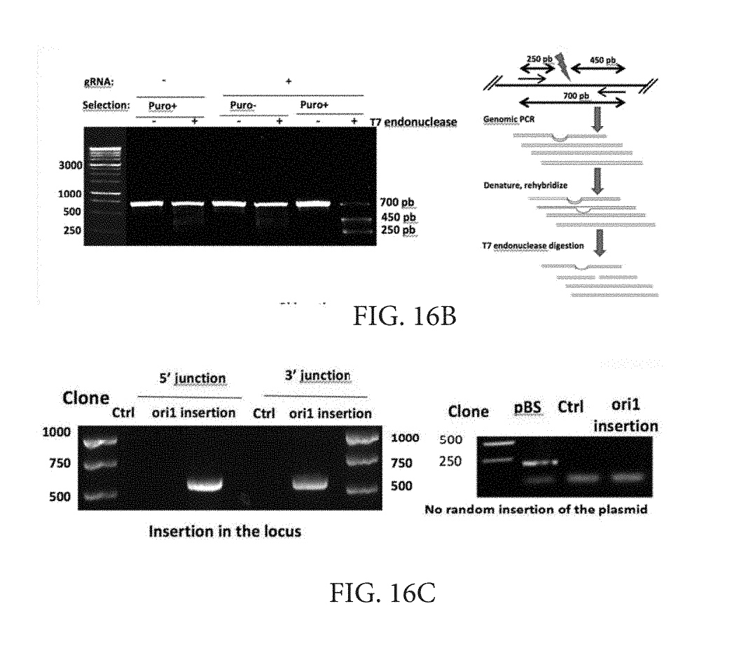

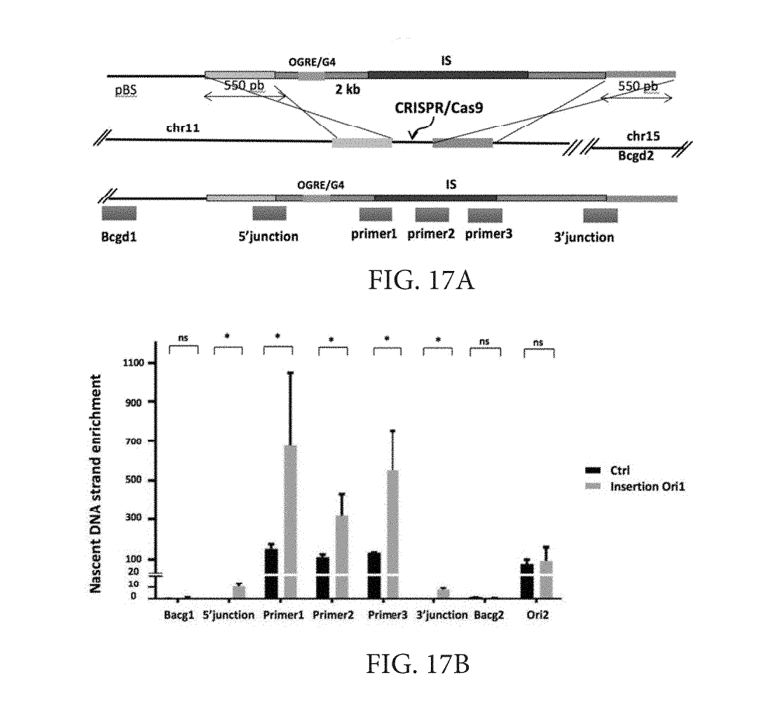

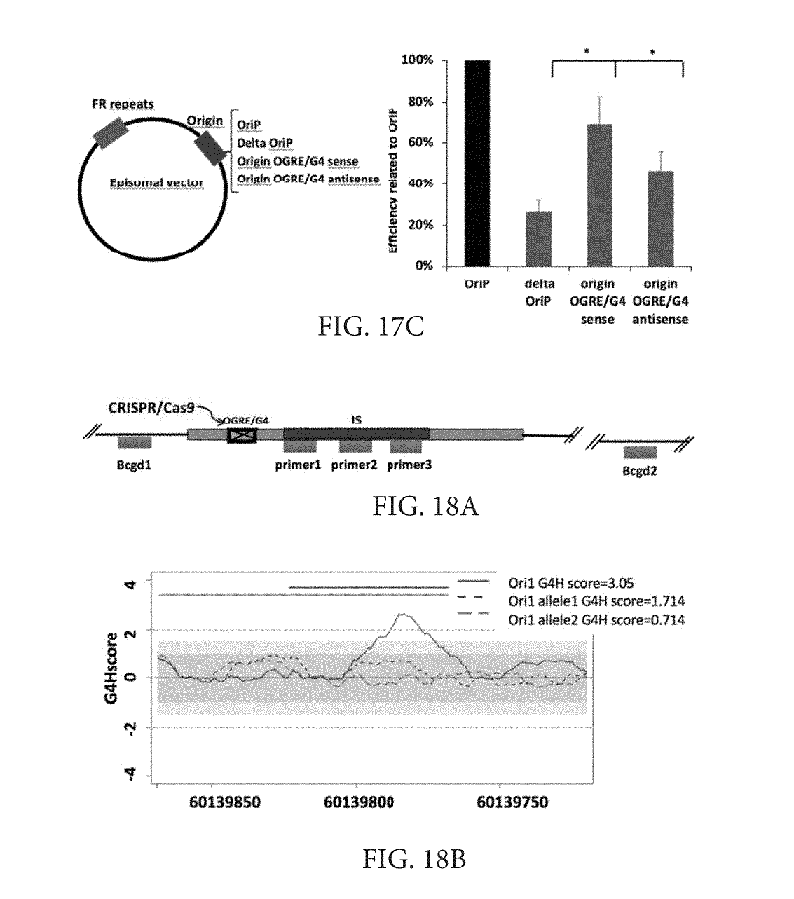

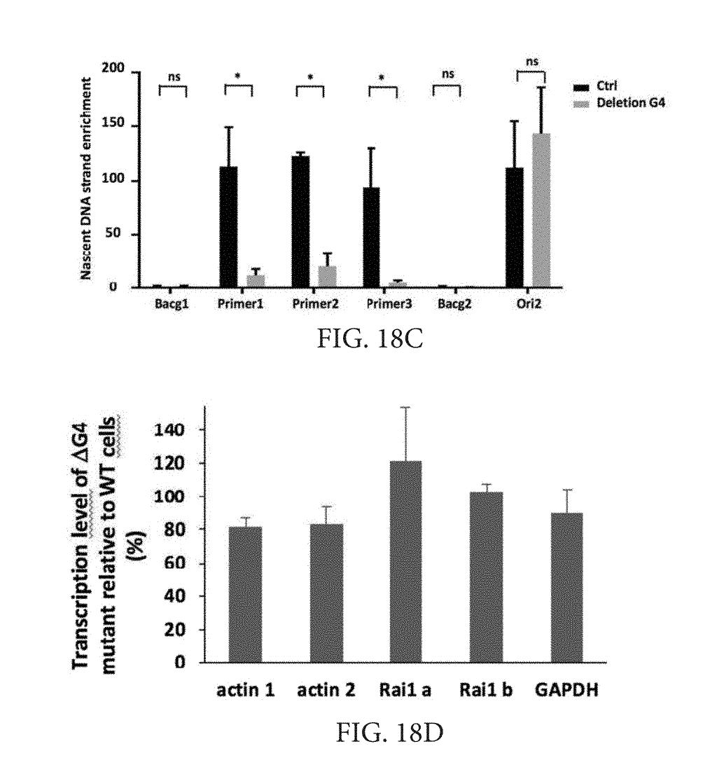

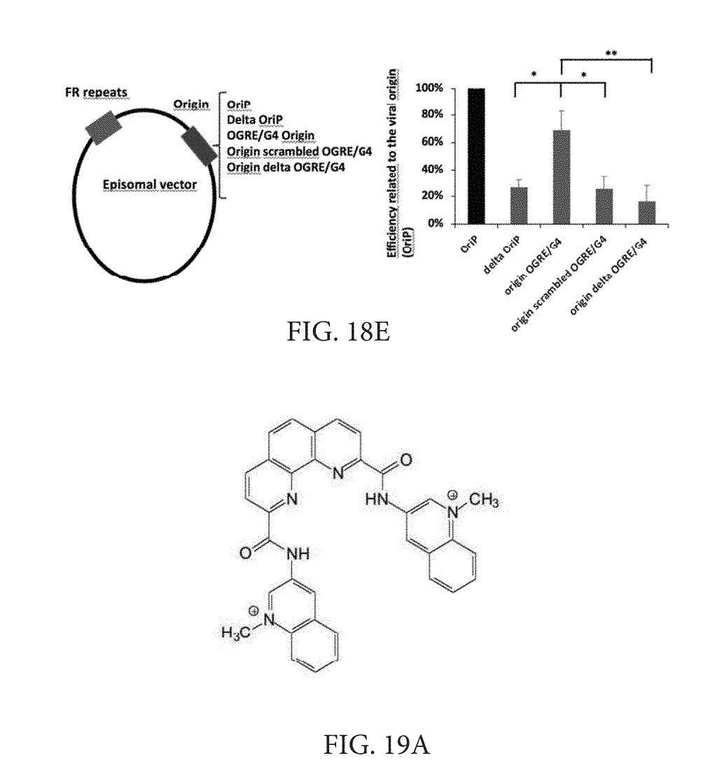

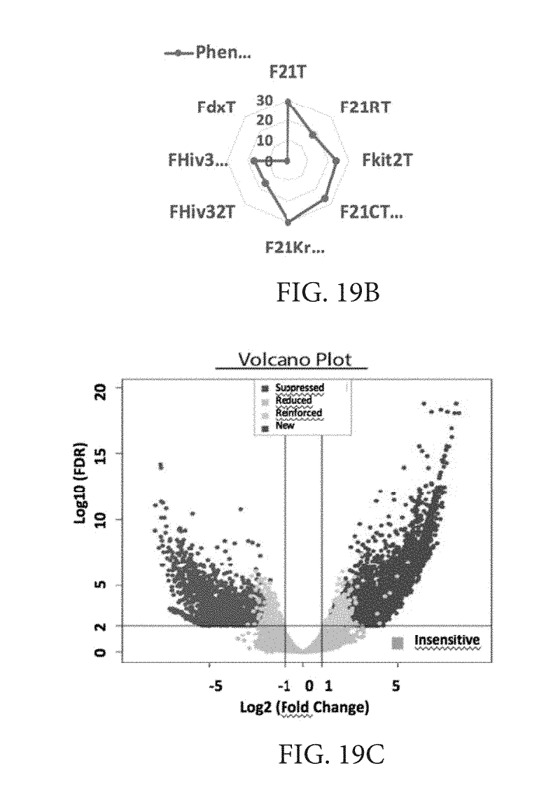

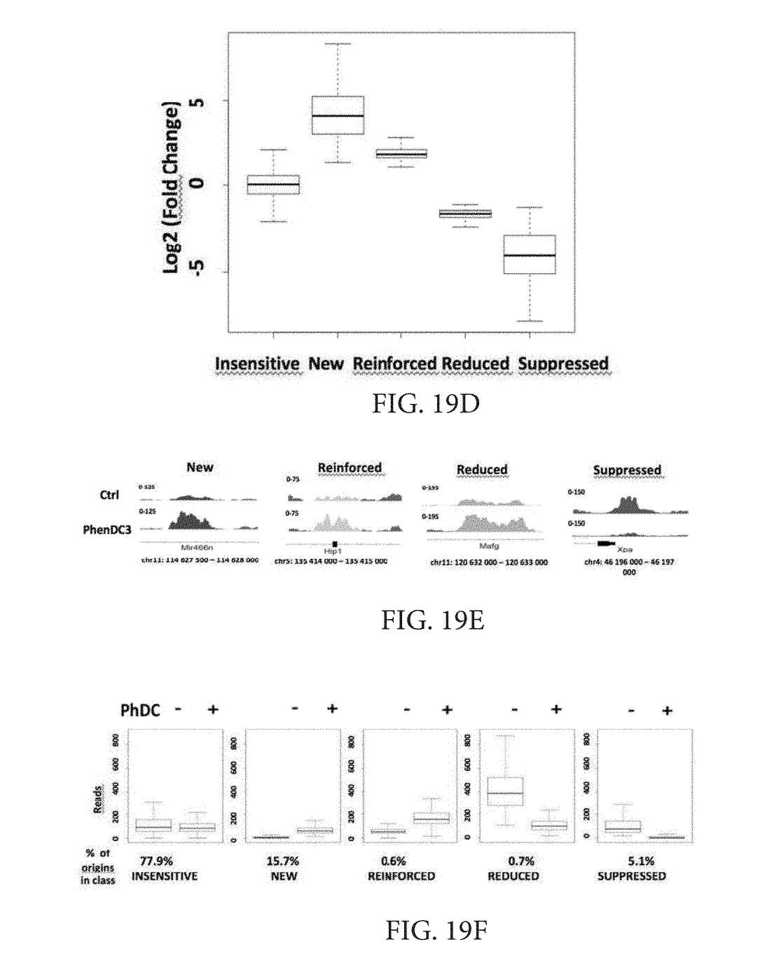

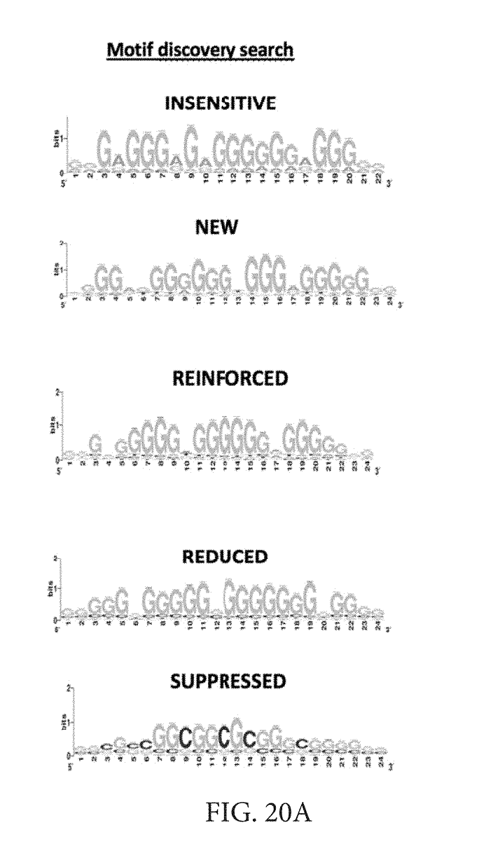

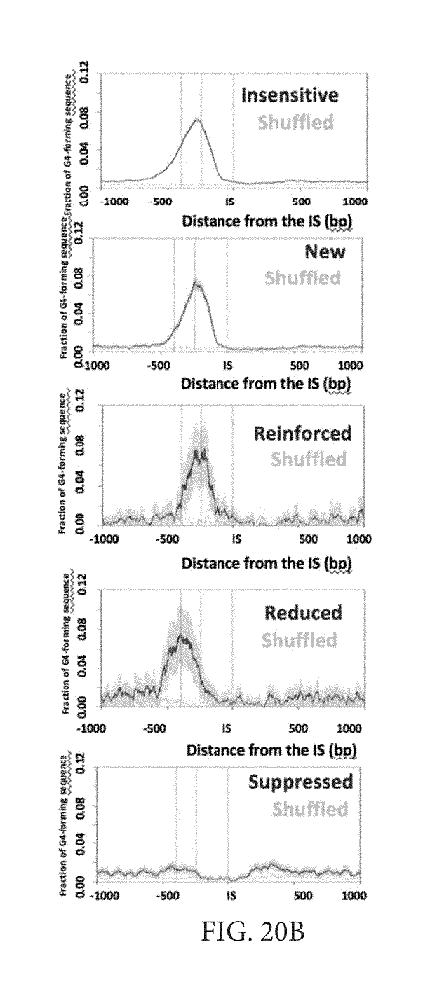

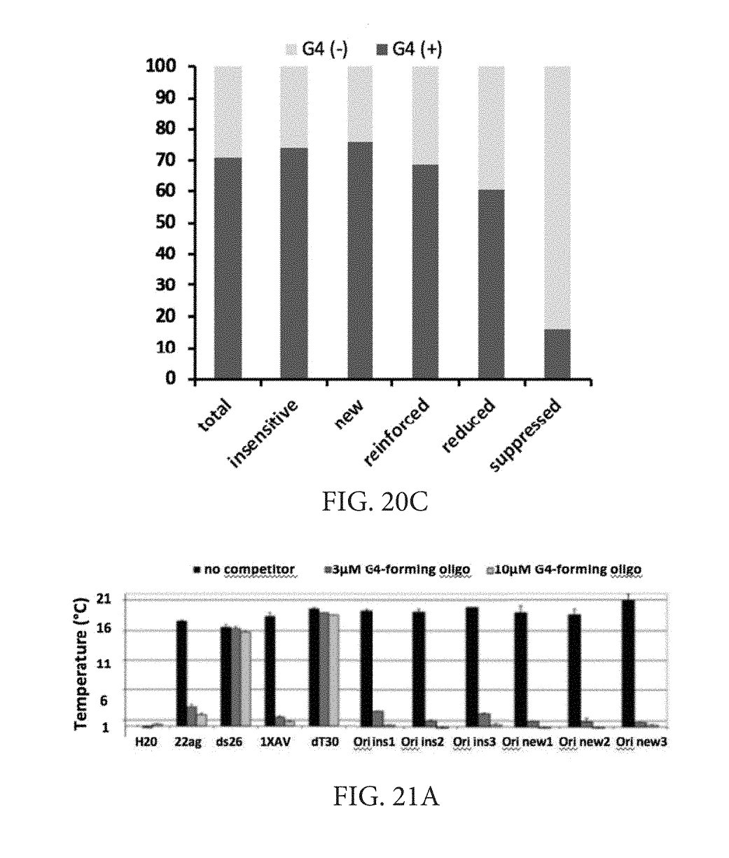

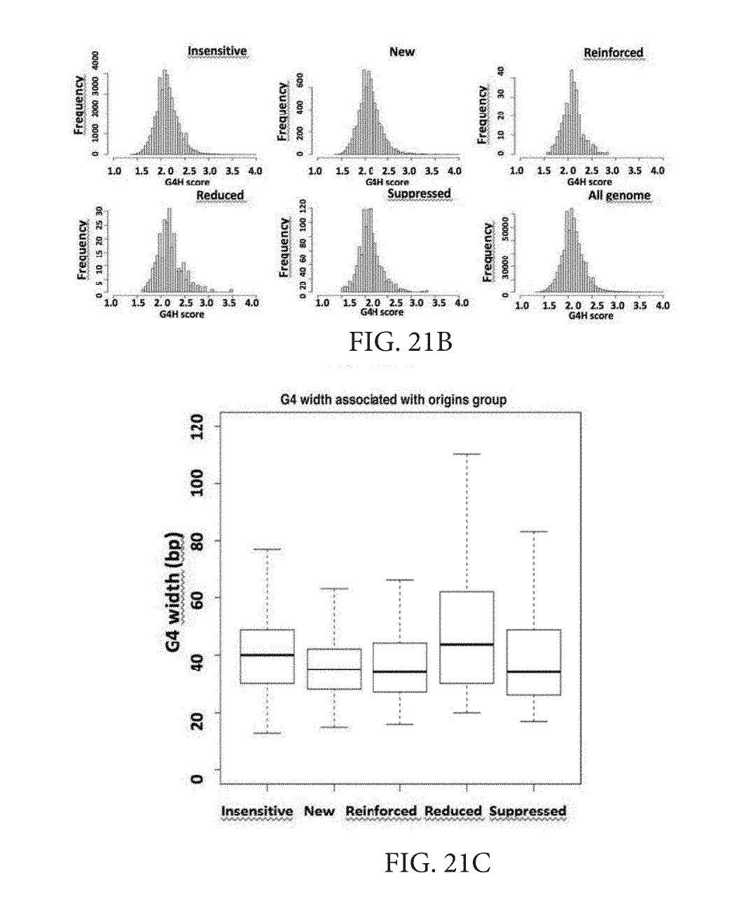

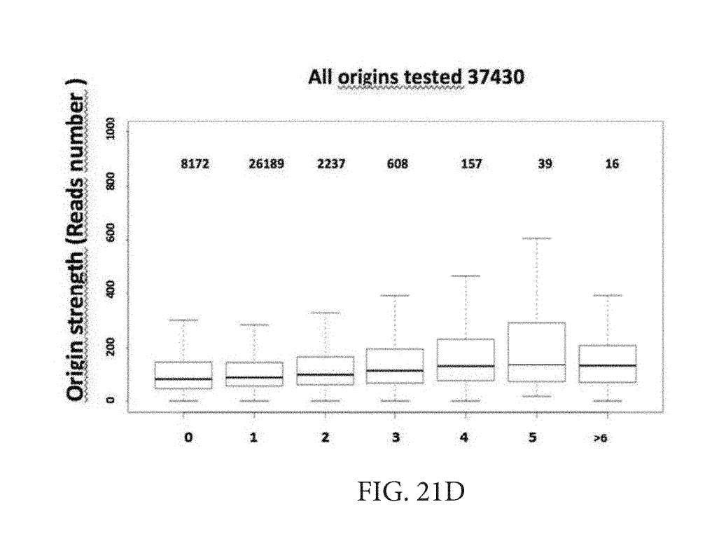

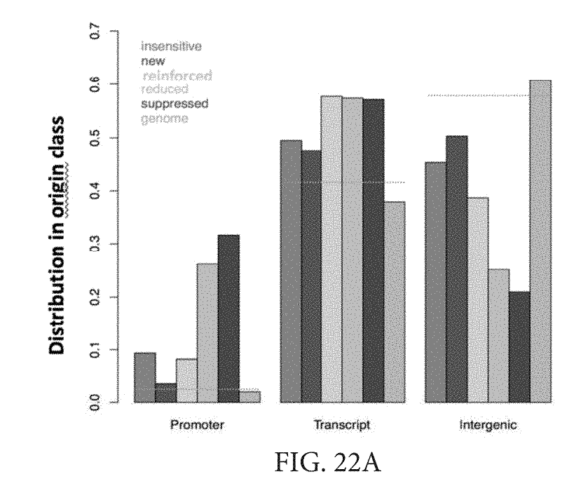

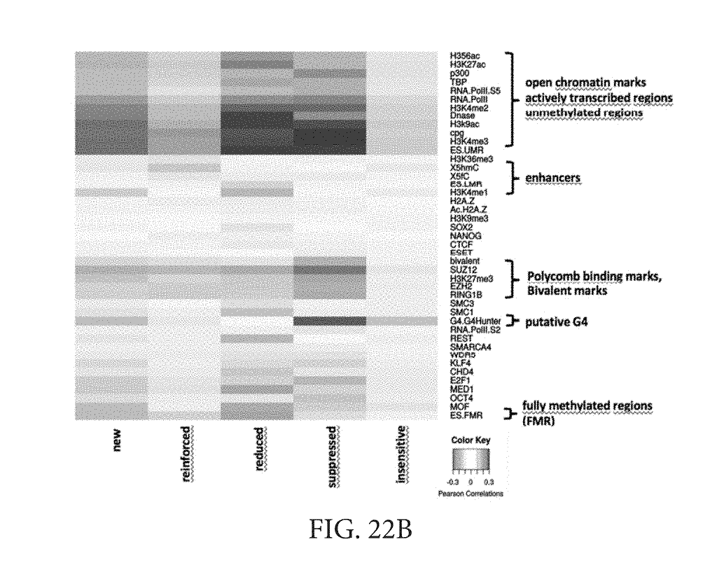

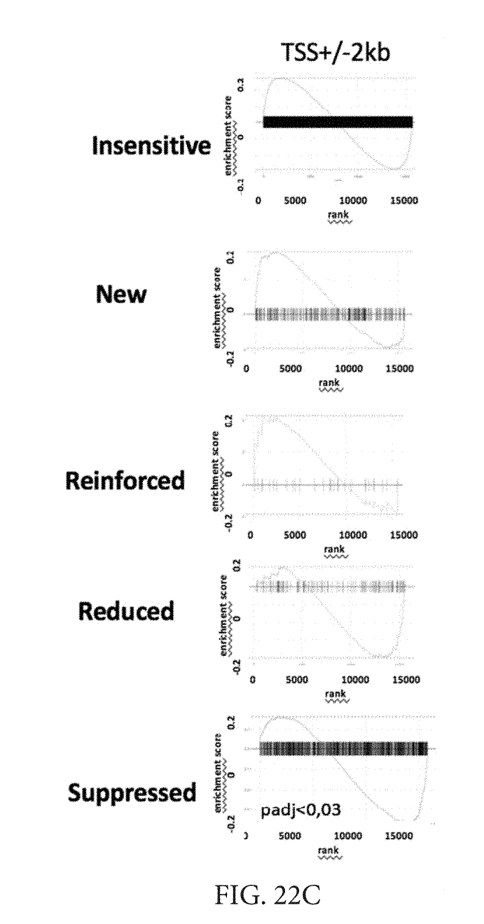

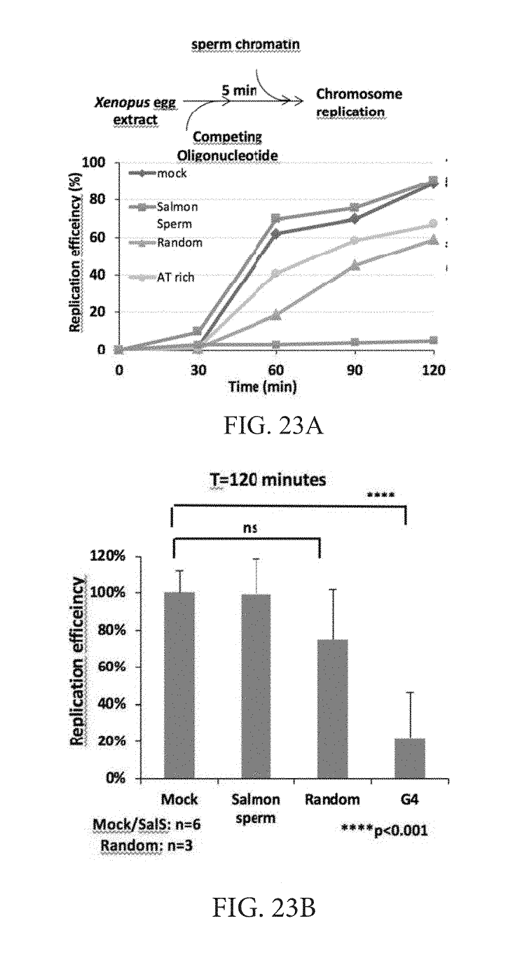

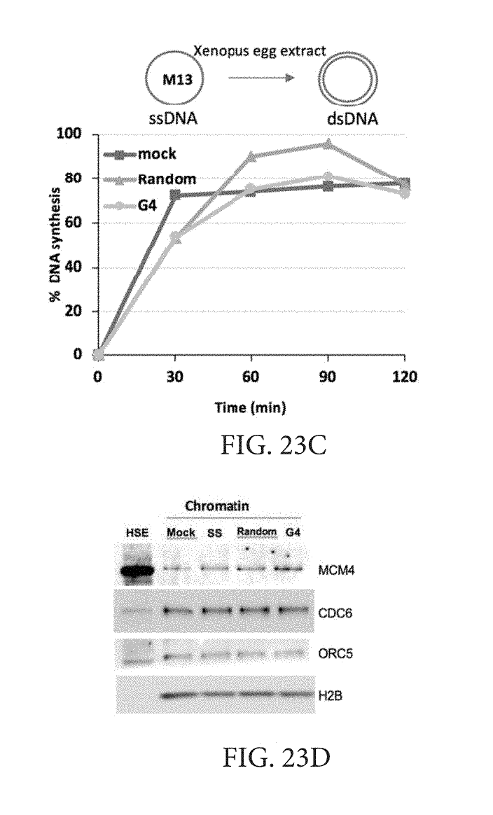

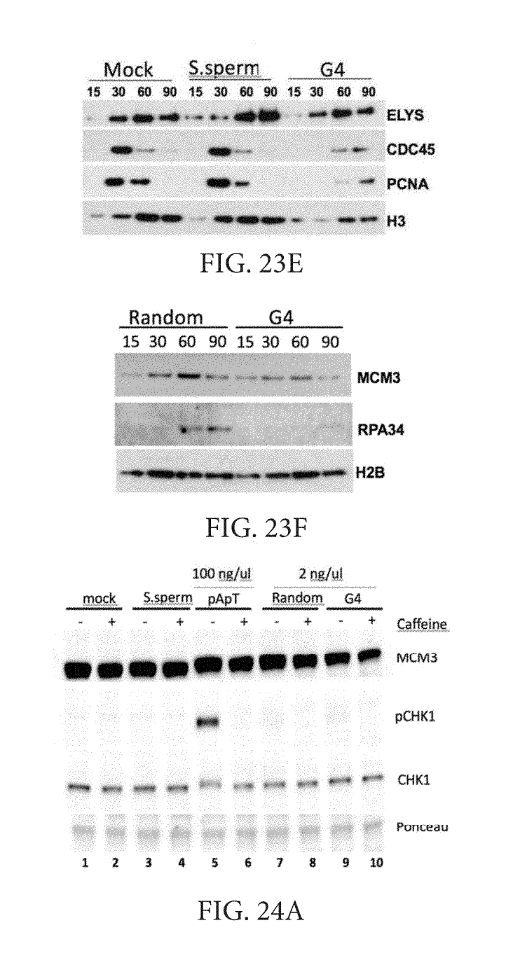

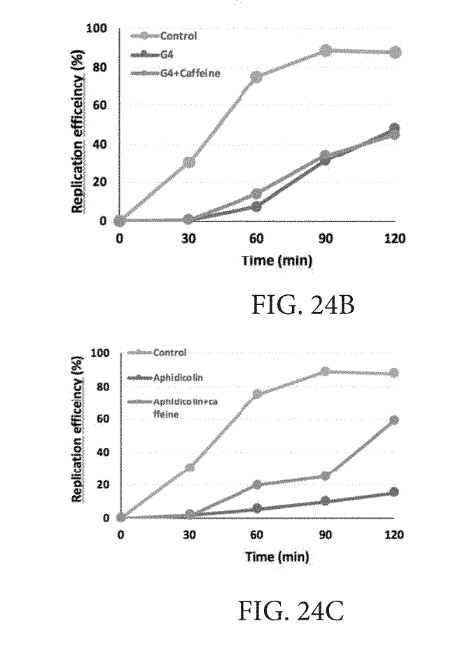

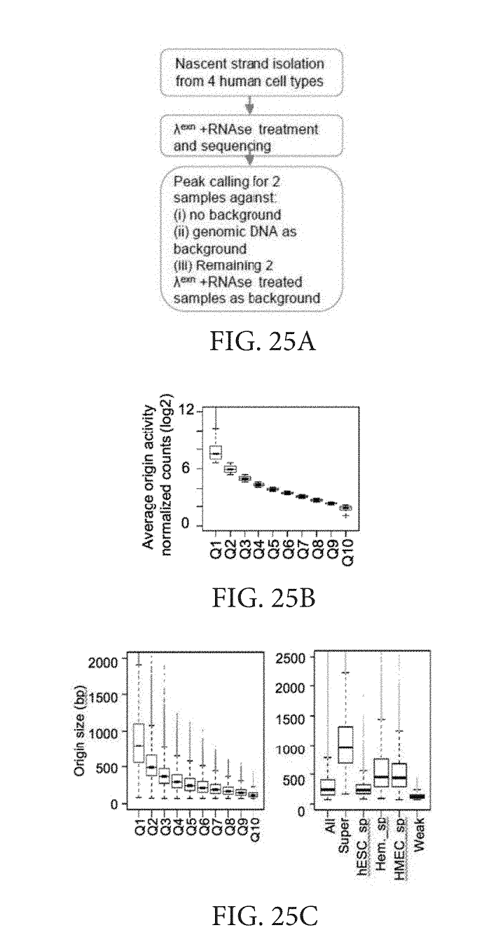

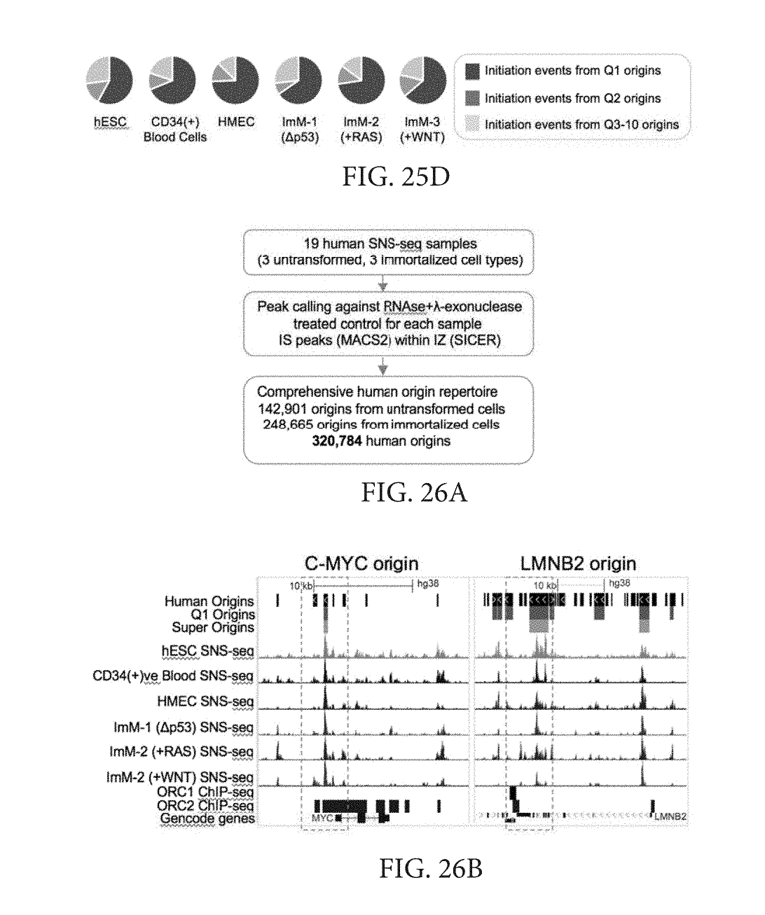

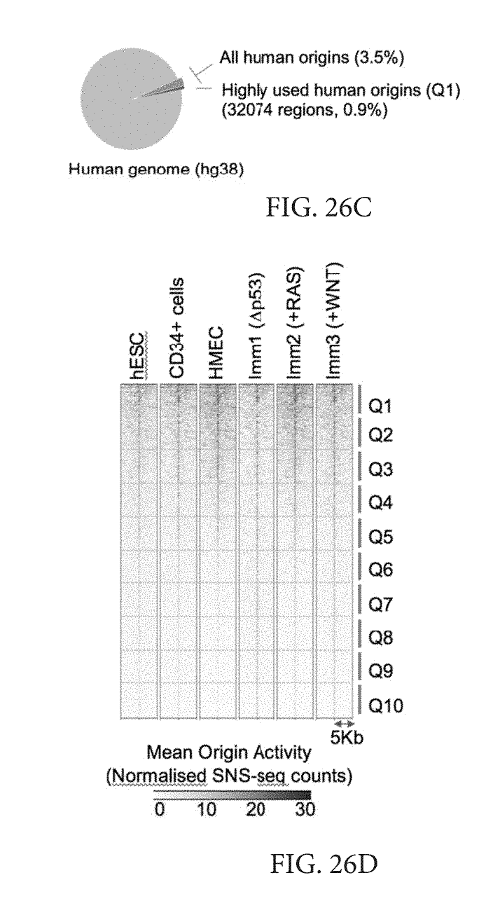

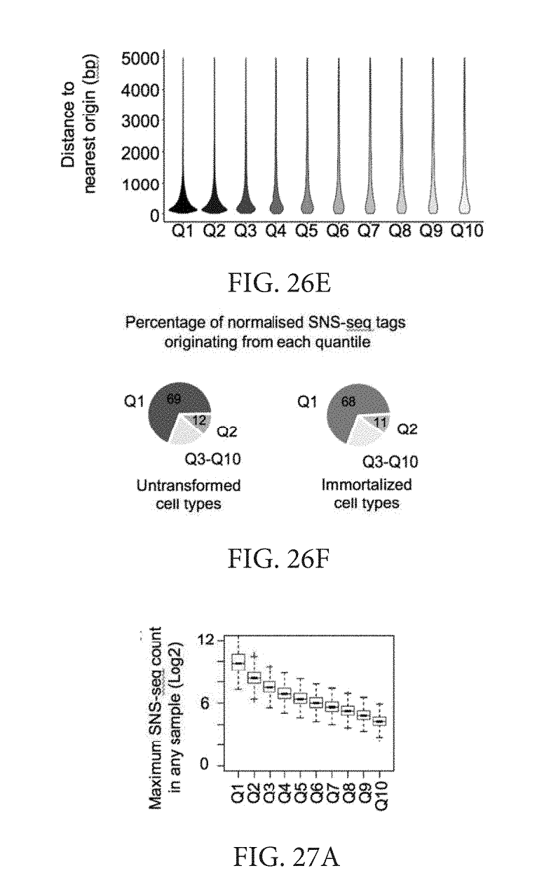

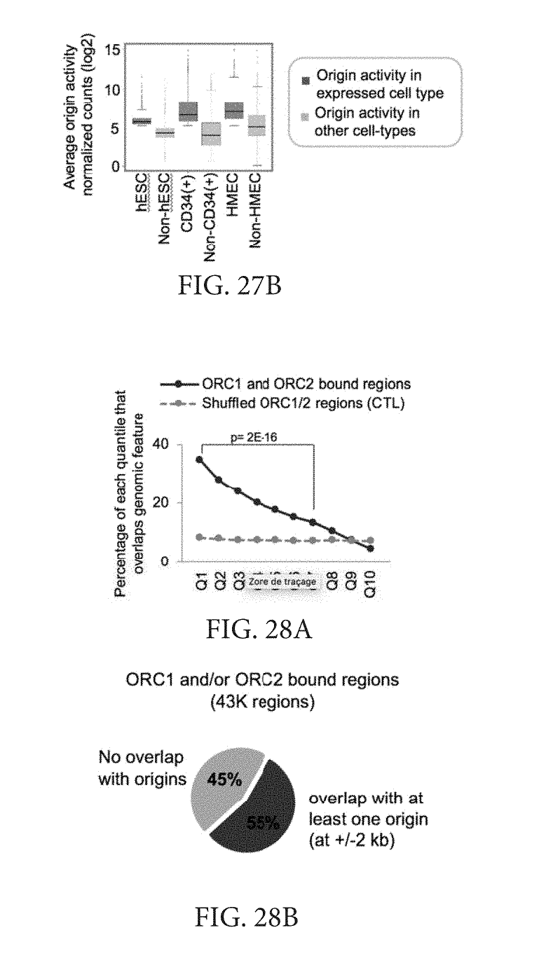

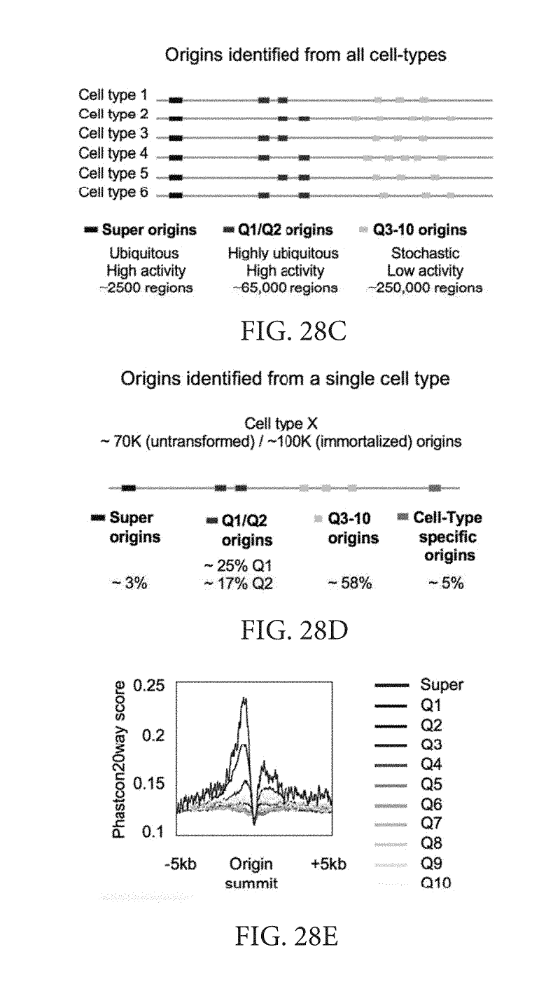

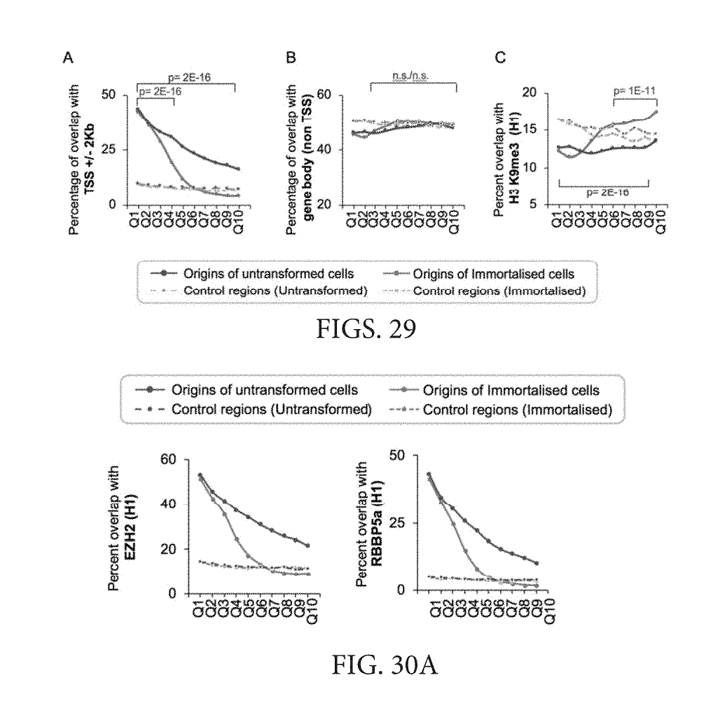

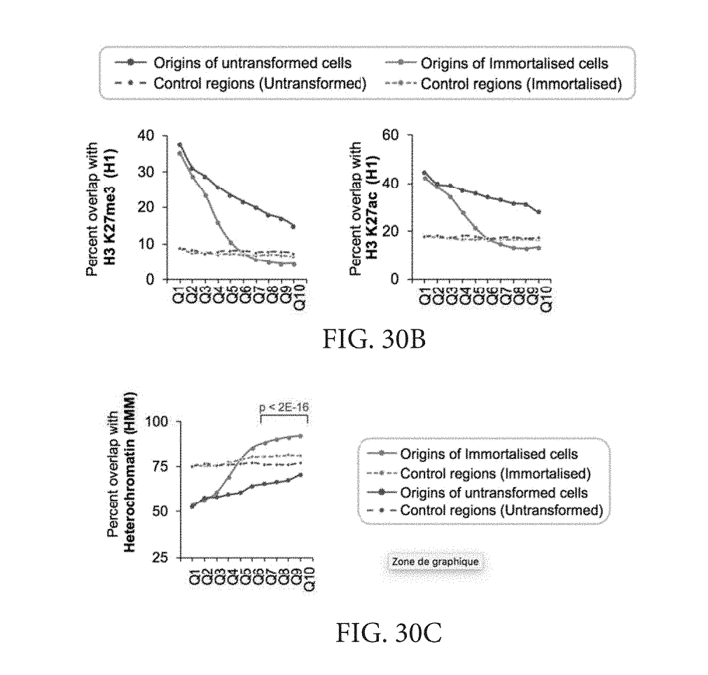

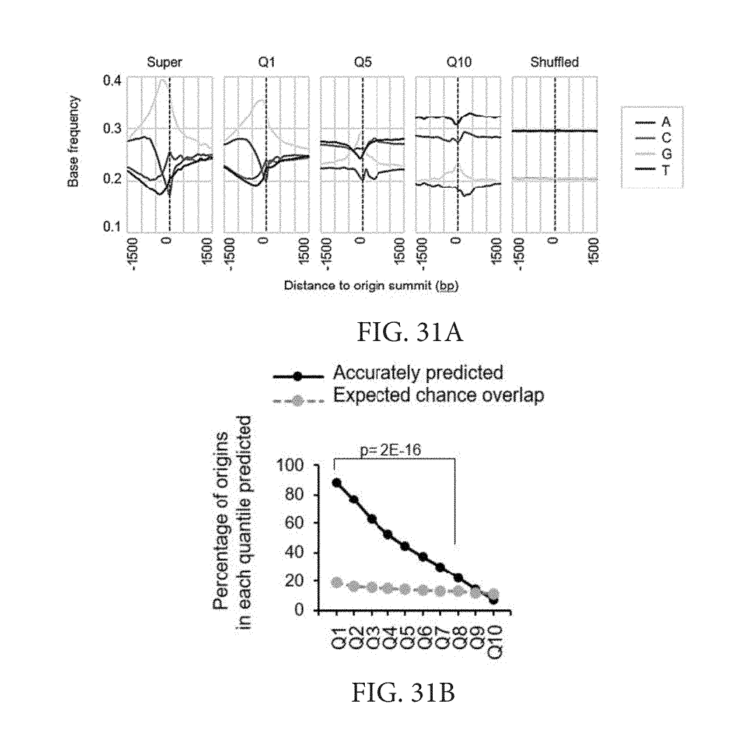

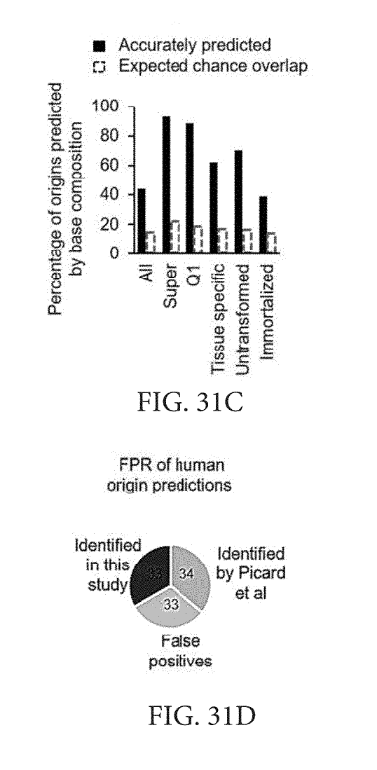

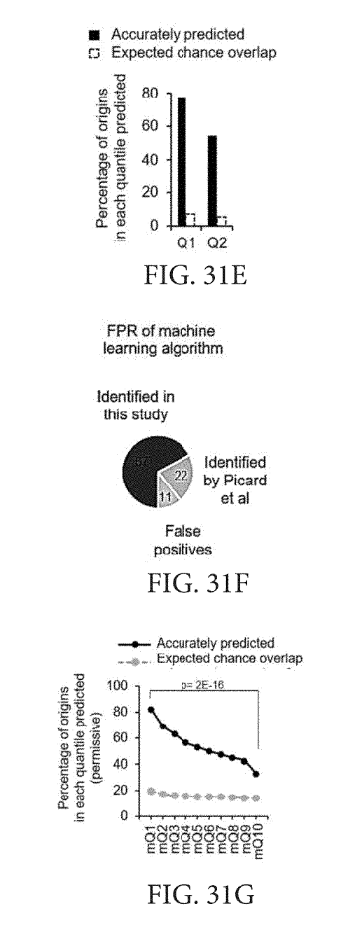

FIG. 15B. Sequence of origins used for genetic modifications in human cells and episomal vector replication.

The potential to form a G4 by each sequence was predicted by G4H and confirmed by a combination of two spectroscopic techniques—thermal difference spectra and circular dichroism. All sequences except their mutated and randomised counterparts exhibit the hallmarks of quadruplex formation.

FIGS. 16A-C Creation of a new replication origin by insertion of an origin containing an OGRE element at an ectopic position in the genome

FIG. 16A. Replication profile of the origin site used, shown in 5 entirely independent replicates of mES cells (left panel). The selected origin is located on chromosome 11 and is associated with a putative G4-forming sequence, that is located 290 bp downstream to the IS. The insertion locus (right panel) is situated in a large origin-, transcription- and G4-free region.

FIG. 16B. Ectopic insertion was obtained by homologous recombination between the linear recombination template and the targeted site in the genome, that was stimulated by a double strand break induced by Cas9 endonuclease expressed in transfected cells. The specificity of a double break (DSB) formation by Cas9 was checked using the Surveyor assay. T7 endonuclease cuts mismatched regions in the dsDNA. A DSB was created at the targeted site only in the presence of a gRNA and when puromycin was used for selection of transfected cells.

FIG. 16C. The correct ectopic insertion to the selected locus was confirmed by PCR detection of the newly created junctions in the genome. Moreover, the absence of any random insertions of pBluescript bearing the insert was confirmed using primers specific to the plasmid.

FIGS. 17A-C. A replication origin containing an OGRE element is functional when inserted at an ectopic position in the genome, as well as on episomal plasmids

(FIG. 17A) An ectopic replication origin (Ori1, containing an OGRE/G4 element center present 290 bp upstream of the IS was inserted by Cas9-stimulated homologous recombination into an origin-free region on chromosome 11 in NIH 3T3 mouse cells. Insertion of a 1907 bp fragment (marked in red) occurred thanks to 500 bp homology arms present on the insertion template (marked in blue and green).

(FIG. 17B) DNA replication activity of the replication origin in the parental cell line (Control in black) and the recombinant cells (in grey). As expected, a two-fold increase in the replication activity was detected comparatively to the parental cell line, whereas external Ori 2 origin exhibited the same replication activity in both cell lines. Note that NS activity was also detected in the 5′ and 3′ junction of the insertion site, but not in the corresponding control regions. Background control regions, bcgd1 et 2, are located in origin-free region.

(FIG. 17C) An EBV episomal plasmid was tested for DNA replication in HEK293 EBNA1 cells using the Dpn1 digestion method and colony counting (ref and Methods). DNA replication activity was assayed either with the OriP origin, or a 500 bp fragment of origin 2 containing an OGRE/G4 element in sense or antisense orientation. SD deviations were calculated from at least 4 independent experiments.

FIGS. 18A-E: G4 structure deletion strongly decreases replication activity of an endogenous origin.

(FIG. 18A) Deletion of the G4 of an endogenous replication origin (Ori1) was obtained using CRISP/Cas9 and the mutation occurrence was checked thanks to a restriction site situated in the vicinity of targeted G4 (see methods).

(FIG. 18B) G4-propensity profiling of origin-associated sequence targeted for deletion. Origin1 wt-sequence is located on chr11 near the 60/139,800 and presents a strong peak on the G4Hunter score profile (red line). Such peak is not present for the sequences of the deleted alleles 1 and 2 (blue and green dotted lines respectively) and no point above 1 or below −1 is observed, arguing against the probability of G4 formation at this locus. The dotted lines in top indicate the extend of the deletions for allele 1 (red and blue) and allele 2 (green and blue).

(FIG. 18C) Nascent strand enrichment of the replication origin in the parental cell line (in black) and in cells containing the G4-mutate origin (in grey). The replication activity is strongly decreased after the mutation, whereas the activity of external origins (Ori 2; right panel) did not vary. Background control regions, bcgd1 et 2 were located in origin-free regions.

(FIG. 18D) Deletion of the G4 did not affect transcription level of the Rail gene, associated with the origin. Transcription efficiency is presented as a ratio of the respective RNA level between wt and G4-deleted cell line. As a control, housekeeping genes were used—actin, GAPDH. For primer positions see Methods.

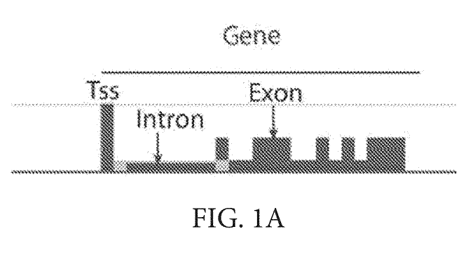

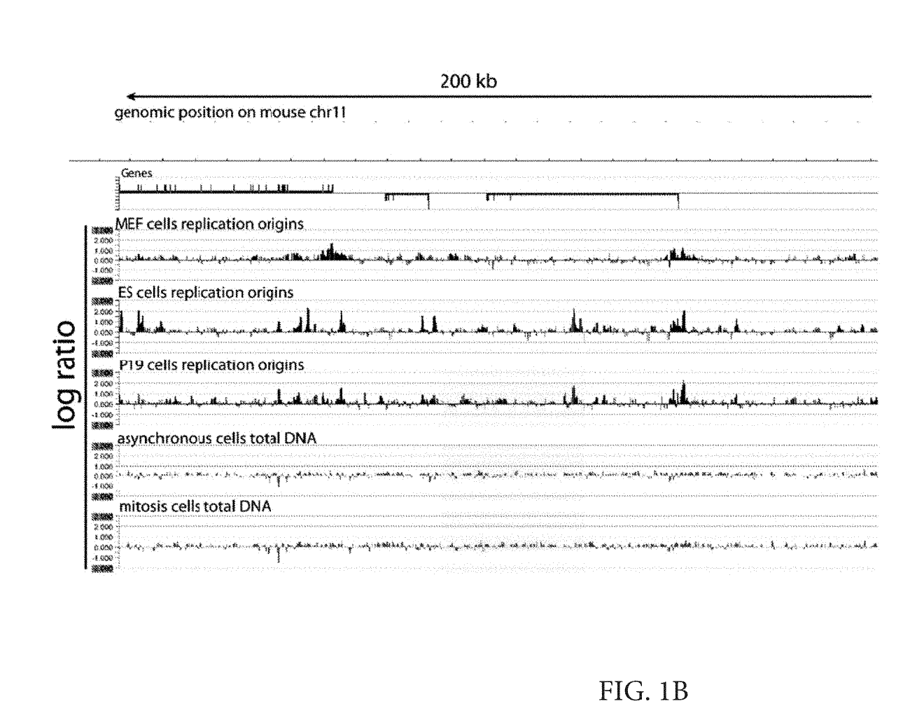

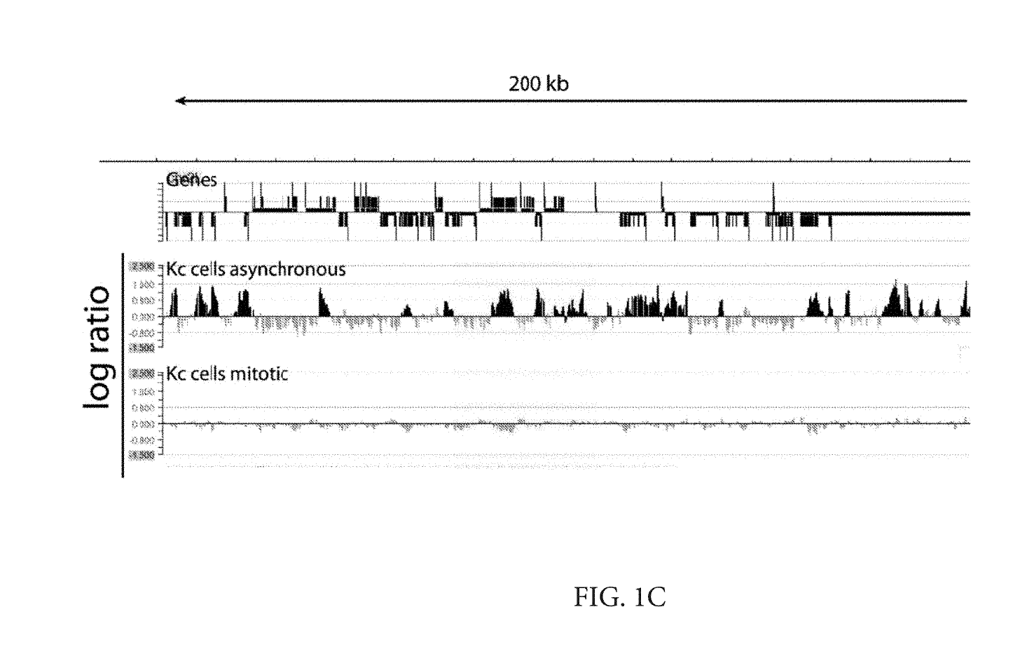

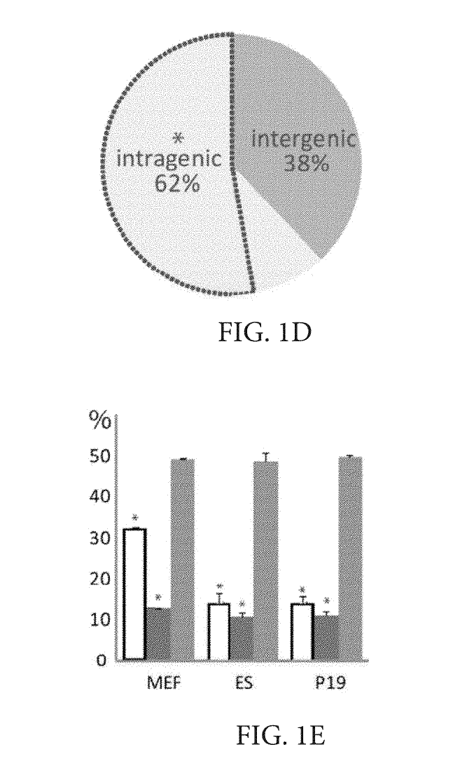

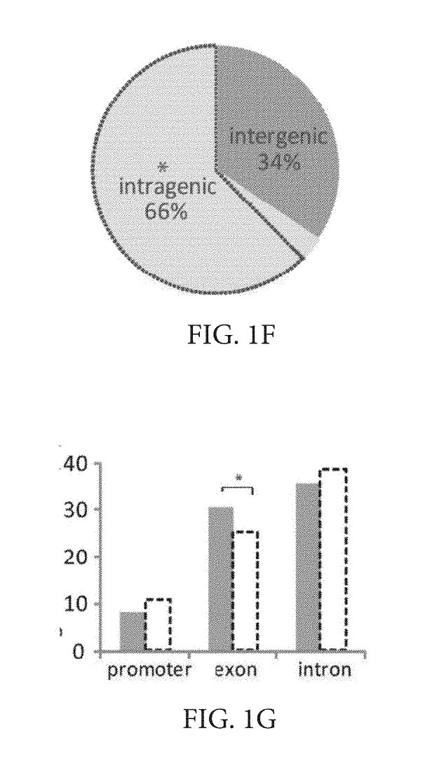

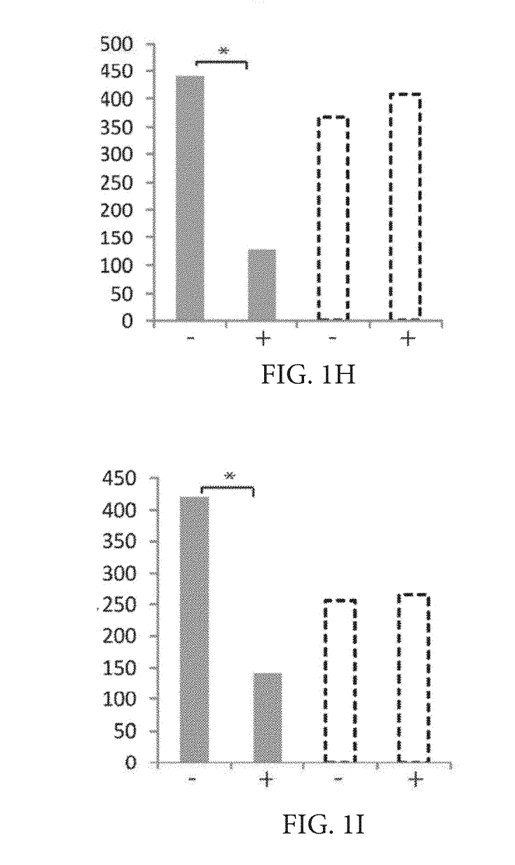

(FIG. 18E) DNA replication activity was assayed as in FIG. 1C either with the EBV origin, or with the 500 bp OGRE/G4 element of Ori 2, or the same sequence with the OGRE/G4 sequence scrambled or deleted. SD deviations were calculated from 4 to 7 independent experiments

FIGS. 19A-F. Changes in the replication origin repertoire upon G4-stabilization by PhenDC3.

(FIG. 19A) Formula of PhenDC.

(FIG. 19B) FRET melting assay: stabilisation effect of PhenDC3 for several G4 sequences labelled with Fam on 5′ and Tamra on 3′. Each G4 sequence was prefolded at 0.2 μM in 10 mM LiCaco pH 7.2 supplemented with 10 mM KCl and 90 mM LiCl before adding the PhenDC3 ligand at 1 μM. Stabilization (increase in T1.2, expressed in ° C.) is plotted for 8 different sequences (7 different quadruplexes and one duplex). The sequences tested were F21T (human telomeric DNA), F21RT (human telomeric RNA), Fkit2T (c-Kit2 human oncogene promoter), F21CTAT (mutant human telomeric DNA), FHiv32T (HIV PRO2 sequence), FHiv321T (HIV PRO1 sequence) and FdxT (intramolecular duplex). PRO1 and PRO2 actually correspond to two different parts of the same HIV promoter region. PhenDC3 shows high affinity for various G4 considered, with no obvious preference for a given topology and can be considered as a “generalist” G4 ligand.

(FIG. 19C) Volcano plot representation of origins affected by PhenDC3 treatment. We identified the universe of bound sites from all NS-seq samples and performed differential binding analysis. For each origin we plotted corrected p-values (false discovery rates, −log 10(FDR)) and the log 2 fold change (FC) of control versus treated samples. The horizontal and vertical lines correspond to thresholds for detecting differential origins. According to fold changes and peak reproducibility we classified differential origins into five different impact group (suppressed, reduced, reinforced, new and insensitive) as described in the material and method.

(FIG. 19D) Fold change (FC) in reads number in control to PhenDC3 treated condition was used to define differential origin classes. No change in the fold change was observed for the class insensitive. The highest positive FC was scored for the class new, following reinforced, reduced and the most negative FC in the class suppressed.

(FIG. 19E) Examples of the activity of the corresponding origins before or after PhenDC3 treatment, with the corresponding genomic regions indicated and origin location colored according to the class it belongs to. (Faire la figure avec les insensitive).

(FIG. 19F) Activity of the replication origins (reads number) in each origin class in control and PhenDC3-treated cells with a distribution of origins between classes.

FIGS. 20A-C. Characterization of genetic elements associated with origin classes

(FIG. 20A) In each affected origin class, except the suppressed class, an OGRE/G4 element was found by a de novo motif discovery using the RSAT suite (that confirms our previous discovery Cayrou et al, 2015).

(FIG. 20B) Fraction of a G4-forming sequence in function of the distance from the IS. The G4 element forms a relatively sharp peak upstream to the IS at an average distance of 250 bp in all origin classes except in the suppressed class. The standard deviation is shown in pink. In yellow and light yellow, the fraction of G4-forming sequence in shuffled regions and their standard deviation is shown, respectively.

(FIG. 20C) G4-association in the different origin classes. Insensitive, new, reinforcedand reduced replication origins are mainly G4-associated in contrast to suppressed origins.

FIGS. 21A-D. G4 properties at the different origin classes after G4-stabilization.

(FIG. 21A) PhenDC3 binds to G4 motif found in selected insensitive and new replication origins. A FRET competition assay is used, in which stabilization (ΔT1/2, in ° C.) of the human telomeric quadruplex F21T by PhenDC3 is provided either without (black bars) in the presence of increasing amounts of each Ori G-rich sequence at 3 or 10 μM strand concentration (dark green and light green bars, respectively). Positive (22Ag, 1XAV, both forming G4 structures) and negative (ds26, dT30 double- and single-stranded, respectively) control sequences are also provided. An efficient competition by a quadruplex forming oligonucleotide is evidenced by a sharp drop in stabilization, all sequenced tested induced this effect, confirming excellent G4-forming capacities.

(FIG. 21B) The distribution of the G4 Hunter score is similar in all defined origin classes and genome-wide for all pG4s.

(FIG. 21C) The length of the potential G4 does not significantly vary in different origin classes of origins.

(FIG. 21D) Replication origin strength (measured In reads number) increased slightly with an increasing number of G4s associated with the tested origin.

FIGS. 22A-C. Transcription and epigenetic landscape related to origin classes.

(FIG. 22A) Genomic localization of origins in different classes with elements relative to transcription. Down-regulated origins ((suppressed and reduced) are mainly located at promoters, in contrast to the origins belonging to the other classes. Random origins are equally distributed in transcription-related regions (dotted lines).

(FIG. 22B) Epigenetic marks association with origin classes. All open chromatin marks tested were found in the vicinity of reduced and suppressed origins. New and reinforced origins were located mainly in highly methylated regions.

(FIG. 22C) GSEA analysis for origins situated in transcribed regions (TSS±2 kb). Origins have a tendency to follow the transcription changes upon the G4-stabilization, the transcription downregulation was found to be significantly correlated with suppressed replication origins (p.adj<0.05), other classes follow similar trend without significant p-values.

FIGS. 23A-F. OGRE/G4 elements compete for ds- but not ssDNA replication, at the activation step of DNA replication.

(FIG. 23A) Scheme of the experiment using competitions by ds oligonucleotides in replication kinetics of sperm nuclei in Xenopus Low-speed Egg Extracts (LSE). The extracts were pre-incubated with the competing oligonucleotide for 5 min at 22° C. Control: LSE incubated in parallel in the same experiment with the same volume of ultrapure H2O.

(FIG. 23B) Average DNA replication efficiency (mean+SD) of nuclei incubated in competing DNA treated extracts (n=6 for mock/salmon sperm DNA preincubated extracts, n=3 for mock/random oligonucleotides). Total incubation time was 2 hrs. Error bar represents standard deviation (SD). P-values were obtained from two tailed Student t-test analysis.

(FIG. 23C) Replication kinetics of ssM13 complementary DNA strand synthesis in HSE that were pre-incubated with the indicated competing DNA or H₂O (mock).

(FIG. 23D) Competition by G4 oligonucleotides does not affect the formation of PreRCs. Sperm nuclei were incubated in mock- or competing oligonucleotides-treated HSE. chromatin was isolated and immunoblotted with the indicated antibodies. The level of histone H2B was used as a control.

(FIG. 23E and FIG. 23F) Competition by G4 oligonucleotides affects the activation of DNA replication. Time-course analysis of replication initiation factors recruitment to chromatin following incubation of sperm nuclei in LSE pre-treated with competing DNA. At the indicated time points, chromatin was isolated and immunoblotted with the indicated antibodies.

FIGS. 24A-C. G4 oligonucleotides do not induce a checkpoint response in Xenopus egg extracts.

(FIG. 24A) Competing ds oligonucleotides were incubated in egg extracts at the same concentration than that used in the study. The checkpoint response was analyzed by western blot using phosphoCHK1 and CHK1 antibodies. pApT oligonucleotides were used as a positive control. Phosphorylation of CHK1 was sensitive to the ATR/ATM kinases inhibitor caffeine.

(FIG. 24B, FIG. 24C) Caffeine, which overcomes pCHK1 dependent inhibition of DNA replication (right graph) did not restore replication of sperm nuclei in presence of G4 ds oligonucleotides DNA (left panel) whereas it can in a control experiment where DNA replication was inhibited by aphidicolin.

FIGS. 25A-D Human DNA replication origins

FIG. 25A. Scheme of controls used for the treatment. Peak calling in the absence of a background reveals more than 200 000 peaks as a mean. The number of peaks is reduced to 140 000 when genomic DNA is used as a background and to 60 000-100 000 peaks if the RNA-primed nascent DNA sample treated with RNAse+λexn is used as a background. The individual values agree with previously published numbers and in all our analyses, we used the RNase treated NS samples as a control for λexo digestion which is for us the more logic one to use in such analyses.

(FIG. 25B) Boxplot representing average normalized SNS-seq counts (Log 2 scale) per quantile. (FIG. 25C) Boxplots comparing the size (in bp) distribution of human origins. The average size in each quantile as well as all, super, tissue specific origins are plotted.

(FIG. 25D) Pie charts representing the percentage of DNA replication initiation events at known origins (Normalised SNS-seq counts) that originate from origins from Q1, Q2 or Q3-10 quantiles in all cell types used in this study.

FIGS. 26A-F: Human origin repertoire

(FIG. 26A) Schematic representation of experimental workflow to define comprehensive human origin repertoire based on 19 human SNS-seq samples across 6 cell types.

(FIG. 26B) UCSC genome browser snapshots of two previously studied human replication origins captured by SNS-seq. Representative SNS-seq read-profiles of hESC, CD34(+)ve blood cell, HMEC and immortalized (ImM1-3) cells are also shown. For reference, previously published ORC1 (black) and ORC2 (red) regions are shown on the bottom as well as Gencode genes (v24). Positioning of all human origins (black), Q1 (brown) and super (orange) origins defined in this study are shown on top.

(FIG. 26C) Pie chart representing the percentage of the human genome (hg38, in grey) which are DNA replication initiation sites (mustard). Q1 origins, 32074 regions, (in brown) occupy only 0.9% of the human genome.

(FIG. 26D) Heatmap of origin activity of all identified human origins (320,748) across 6 different cell-types. Origins are sorted from highest average activity to lowest average activity based on number of normalized SNS-seq reads. Human origins are divided into 10 equal-size quantiles (Q1-Q10) with 32,074 origins in each.

(FIG. 26E) Violin plots comparing the distribution of distance to nearest origin for origins belonging to different quantiles. Q1 origins display the highest accumulation on the bottom, suggesting localization in origin-dense regions.

(FIG. 26F) Pie chart representing the percentage of DNA replication initiation events at known origins (Normalised SNS-seq counts) that originate from origins from Q1, Q2 or Q3-10 quantiles in both untransformed (left panel, blue) and immortalized (right panel, rose) cell types.

FIGS. 27A and B: Higher activity origins display higher ubiquity across replicates and cell types

(FIG. 27A) Origins from higher quantiles (Q5-10) have low activity across samples. Boxplot representing the maximum normalized SNS-seq read count of origins in each quantile in any given sample (Log 2 scale).

(FIG. 27B) Boxplot representing the activity (normalized SNS-seq counts) of cell-type specific origins, both in the cell type (mustard) and in other cell-types (light green).

FIGS. 28A-E: Correlations with ORC binding sites and other properties (FIG. 28A) Graphical representation of the percentage of origins (in red) in each quantile that overlap the ORC1/2-bound regions (within +/−2 Kb of the ORC-peak) and control regions (in gray, obtained as described above). p-value for significance in this figure is obtained using Chi-square Test of Goodness-of-Fit in R with observed and expected values for overlap.

(FIG. 28B) Pie chart representing the percentage of ORC-bound regions that overlap DNA replication initiation sites as determined by SNS-seq.

(FIG. 28C) Schematic representation of ubiquity vs consistency in of placement of human origins. Super origins are constitutively active in all cell types and replicates examined. Q1/Q2 origins are active in, on average, half the replicates examined whereas Q3-Q10 origins are usually active in less than three replicates examined.

(FIG. 28D) Schematic representation of origins belonging to different quantiles identified in a single cell-type. On average, ˜40% of origins in a single cell type belong to Q1/Q2 origins and the remaining origins belong to Q3-Q10 origins. It is worth noting that super and tissue-specific origins make up approximately 3% and 5% of the origin repertoire of a single cell type.

(FIG. 28E) Super and Q1 origins have conserved sequences upstream of the initiation site. Graph represents averaged Phastcon20scores of human origins (Super, Q1-Q10), centred on the origin summit with 5 Kb flanking regions on each side.

FIG. 29 Weak origins from immortalized cell types are depleted from open chromatin and are associated with heterochromatin Percentage of origins in each quantile (untransformed Q1-10 in blue, immortalized Q1-Q10 in rose) that intersect TSS (+/−2 kb) or (32B) gene body (excluding TSS+2 kb; non-TSS) of Gencode (v25) genes. (32C) Percentage of origins in each quantile that overlap regions with heterochromatin-associated histone mark H3K9me3 in H1 (hESC) cells. p-value for significance in this figure is obtained using Chi-square Test of Goodness-of-Fit in R with observed and expected values for overlap.

FIG. 30 A-C Association of human origins with epigenetic marks

Association with EzH2 and RBPP5a sites (FIG. 30A) H3K27me3 sites, and H3K27ac (FIG. 30B), heterochromatin (FIG. 30C).

FIGS. 31A-G: Mammalian origins can be predicted based on DNA-sequence alone

(FIG. 31A) Base content of regions flanking human DNA replication initiation sites and shuffled control regions. Frequency plots are centered at origin summits (highest point of read pile-up). Base frequency represents the proportion of each base in sliding windows of 100 bp, on a scale of 0 to 1.

(FIG. 31B) Percentage of origins in each quantile that overlap predictions (in black) based on genome scanning algorithm and randomly shuffled matched-size regions (in gray).

(FIG. 31C) Barplot representing the percentage of origins that can be predicted by permissive criteria for specified set of origins (for all comparisons, p<2E-16).

(FIG. 31E) Accuracy of predictions based on a machine learning algorithm that considers the CC, CG and GG content of candidate regions.

(FIG. 31G) Percentage of mouse origins in each quantile that overlap predictions based on predicted regions (in black) and randomly shuffled matched-size control regions (in gray). p-values for significance in this figure are obtained using Chi-square Test of Goodness-of-Fit in R with observed and expected values for overlap.

### EXAMPLES

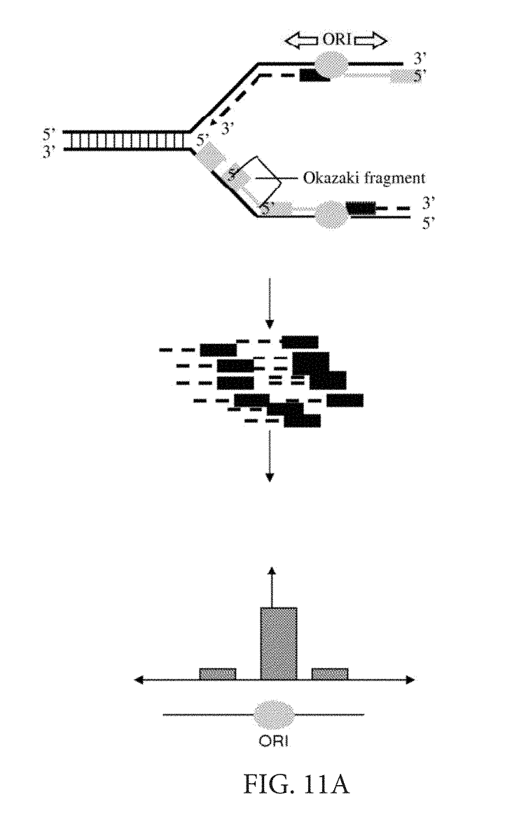

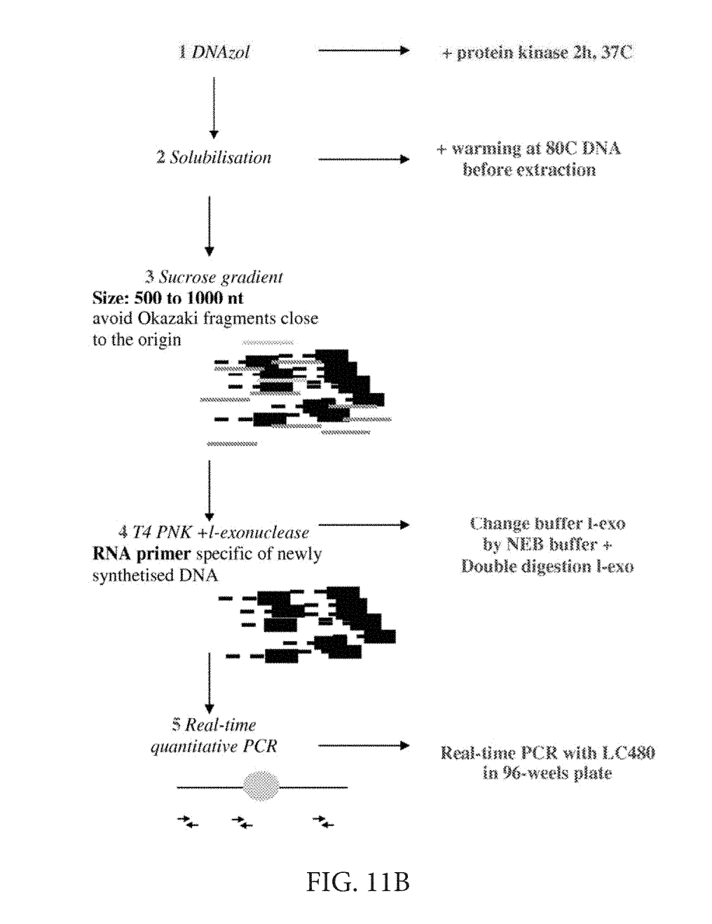

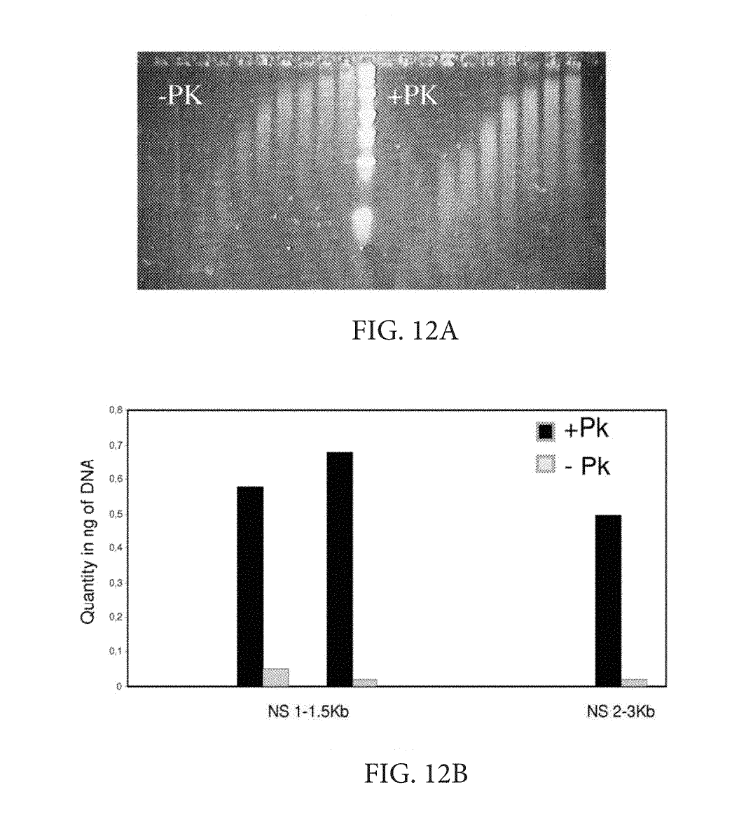

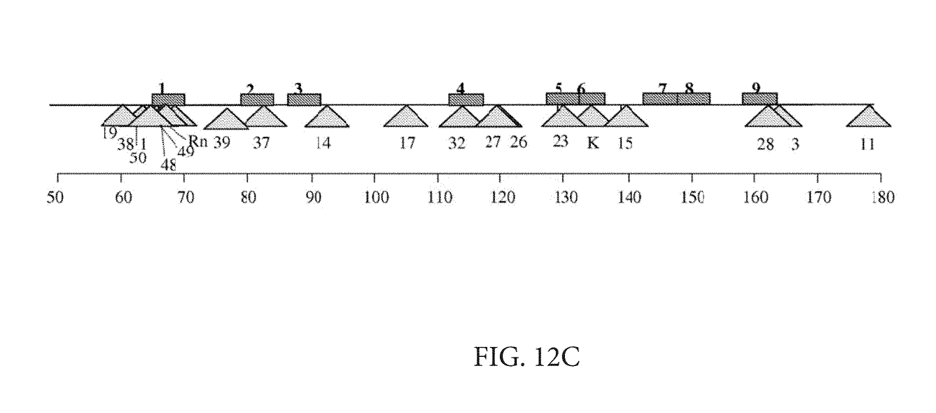

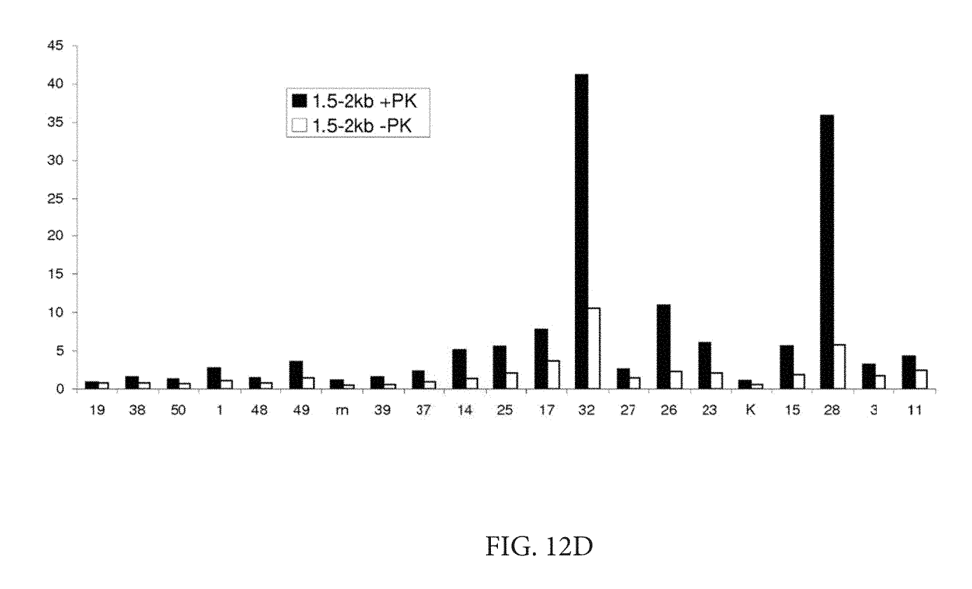

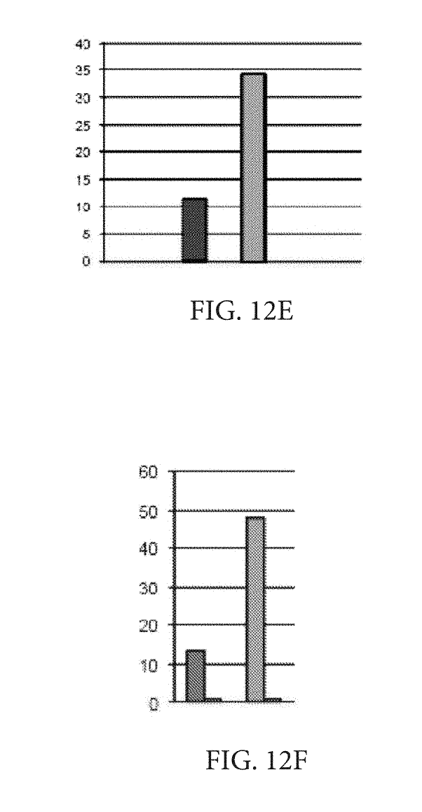

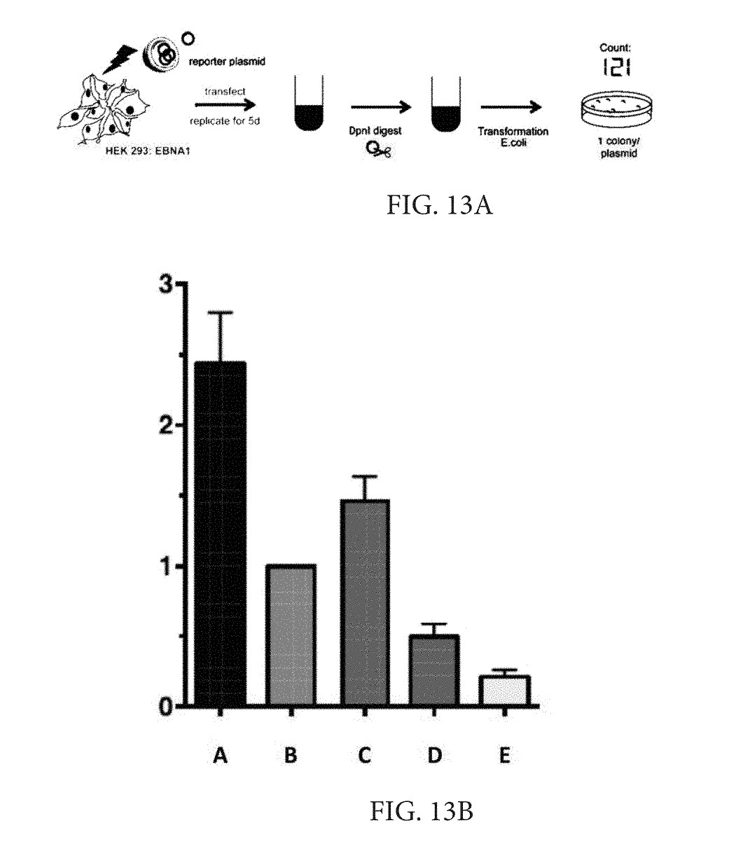

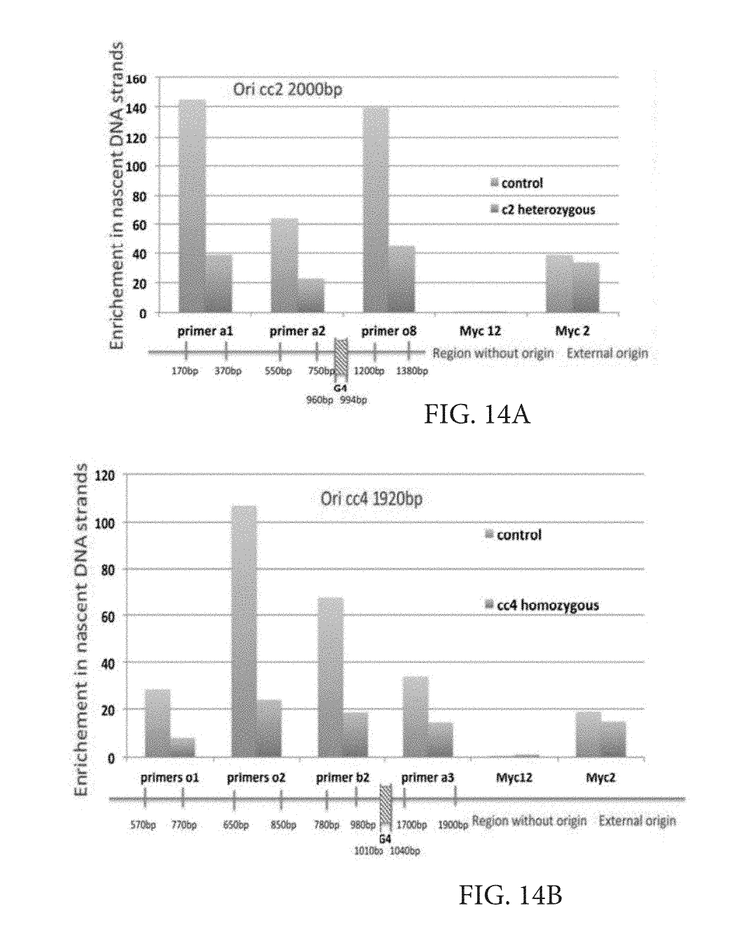

### Example 1: Protocole for Nascent DNA Purification (FIGS. 11 and 12)

Precipitation DNA
dividing cells (2.5×108 to 5×108=2*150 mm) were washed with PBS.
cells were harvested and lysed in 15 ml of DNAzol® for 5 min at room Temperature (RT)
Proteinase K was added in DNAzol to 200 μg/ml, and incubated at 3 7° C. 2 hours. Centrifugation at 4000 RPM, 15 min and the supernatant is rescued.
To the supernatant, 15 ml of ethanol 100% were added to precipitated for S min at RT.
Spooled out the DNA using a drawn pasteur pipette in a tube with 5 ml of ethanol 70% for 5 min at RT
spooled out the DNA using a drawn pasteur pipette in a new dry tube 2 ml to dry the pellet (30 min at RT).

DNA is resuspended in 2 ml of TEN20 at 70° C. tris 10 mM pH7.9 final
EDTA 2 mM final
NaCl 20 mM final
SDS 0.1%
RNasin 1000 U

The solution was boiled for 10-15 mM, chilled on ice

Sucrose Gradient NS Purification

Load 1 mL onto a single neutral 5 to 30% sucrose gradient prepared in TEN500 in a 38.5-ml centrifuge tube. tris 10 mM pH7.9 final
EDTA 2 mM final
NaCl 300 mM final

Gradients were centrifuged in a Beckman SW28 rotor for 20 h at 24 000 rpm at 4° C.

1 ml Fractions were withdrawn from the top of the gradient using a wide-bore pipette tip 50 μl of each fraction was run with appropriate size markers on a 2% alkaline agarose gel, ON at 4° C. at 40-50 volt.

neutralized gel with TBE1 X and stained with GelRed.

Fractions corresponding to 0.5-1 kb, 1-1.5 kb, 1.5-2 kb and 2-3 kb were rescued and precipitated with 2.5 Vol of ethanol 100% 15 min at −80° C.

Pellets were washed with I ml of ethanol 70% and resuspended in 20 μl of water with 100 U of RNasin.

DNA Contaminant Withdrawn

1—After addition of 2 μl Buffer PNK (New England Biolabs), fractions were boiled for 5 min, chilled on ice,

2—phosphorylation with T4 polynucleotide kinase in a volume of 1 OO μl final vol T4 mix:

| | |
|---|---|
| water | qsp 80 ul |
| Buffer PNK NEB l0X | 1× |
| ATP | 50 nM |
| T4 PNK | 20 U (2 ul of 10 U/ul) |

The reaction is incubated at 37° C. for 1H, 15 min at 75° C. and directly precipitated by ethanol (2.5 vol)-Na-acetate (0.3M) for 15 min at −80° C.

3—Pellets were washed with 1 ml of ethanol 70% and resuspended in 50 μl of water with 100 U of RNasin.

4—The remainder is digested with 5 μl of lambda exonuclease in a final volume of 100 μl Lambda exo mix:

| | |
|---|---|
| water | qsp 50 ul |
| L-exo buffer 1 OX | 1× |
| L-exo (Fermentas 20 U/μl) | 5 ul |
| BSA | IX (l ul of l00X) |

Fermentas L-Exo Buffer

67 mM glycine-KOH (pH 9.4)

2.5 mM MgCl2

50 μg of bovine serum albumin per ml)

The reaction is incubated overnight at 37° C.

Aliquots of both the digested DNA and the undigested control were run on an 2% agarose gel.

5—the nascent strands were extracted once with phenol/chloroform/JAA and once with chloroform/JAA, and ethanol (2.5 vol)-Na-acetate (0.3M) precipitated for 15 min at −80° C.

6—Pellets were washed with 1 ml of ethanol 70% and resuspended in 20 μl of water.

7—The NS is subjected to another step of phosphorylation by T4 PNK and lamda-exo digestion (2- to 5-)

8—The final NS resuspended in 50 μl of tris 10 mM is directly quantified with Roche-LC480.

### Example 2: Nascent Strands Amplification

Purification of Nascent Strand with Cyscibe-GFX kit

Elution in 50 ul

use 10 ul and amplify with WGA-Sigma kit without the first fragmentation step.

Purify amplicons with nucleospin kit with a 1/5 dilution in NBA buffer prior to fix on column.

Elution in 50μ1.

LC480 (Light cycler 480) on 0.1 a 0.5 ul of the amplicon.

### Example 3: Genome-Wide Analysis of Replication Origins in Five Different Cell Types Reveals Several Choices but a Conserved Repeated Element

### INTRODUCTION

In metazoans, thousands of chromosomal sites are activated at each cell cycle to initiate DNA synthesis and permit total duplication of the genome. They all should be activated only once to avoid any amplification and maintain genome integrity. How these sites are defined remains elusive despite considerable efforts trying to unravel a possible replication origin code. In Saccharomyces cerevisiae, DNA replication origins are specifically identified by specific DNA elements, called Autonomous Replication Sequence elements (ARS), which have a common AT-rich 11 bp specific consensus. However, sequence specificity identifies but not determines origin selection. Indeed, of the 12,000 ACS sites present in S. cerevisiae genome only 400 are functional [Nieduszynski C A, et al. Genes Dev. 2006 Jul. 15; 20(14):1874-9]. In S. pombe, ARS elements were also identified but they do not share a specific consensus sequence like in S. cerevisiae. Here, DNA replication origins are characterized by AT-rich islands [Dai J, et al. Proe Natl Acad Sci USA. 2005 Jan. 11; 102(2):337-42; Heichinger C, et al. EMBO J. 2006 Nov. 1; 25(21):5171-9] and poly-dA/dT tracks.

In multicellular organisms, it was more difficult to identify common features of DNA replication origins. No consensus sequence element has been found, which can have predictive value, although specific sites are recognized as DNA replication origins in chromosomes of somatic cells. It was soon suspected that metazoan ORis might be linked to other genetic features of complex organisms as the requirement to coordinate DNA replication not only with cell growth but also cell differentiation, and correlations with transcription and/or chromatin status have been found [Cayrou C, et al. Chromosome Res. 201 O January; 18 (1): 13 7-45]. However, identification of replication origins has been hampered by the lack of a genetic test as the ARS test in yeast, and methods to map replication origins which were not always adapted to a robust genome-wide analysis. First recent genome-wide studies to map origins in mouse and human cells (Cadoret et al., 2008; Sequeira-Mendes et al., 2009) have observed a correlation with unmethylated CpG islands regions as well as some overlap with promoter regions [Sequeira-Mendes J, et al. PLoS Genet. 2009 April; 5(4):e1000446]. However, it is not clear whether CpG islands are here a specific mark of replication origins or of the associated transcription promoters.

The Inventors tried to reveal new features of eukaryotic origins, first by upgrading the method used to map nascent stands DNA at origins to a specificity and reproducibility compatible with a genome-wide analysis compatible with the use of tiling arrays, then in a further upgrade compatible with using Next Generation High throughput sequencing The Inventors first used this method for four kinds of cell systems: mouse embryonic stem cells (ES), mouse teratocarcinoma cells (P19), mouse differentiated fibroblasts (MEFs), and Drosophila cells (Kc cells). The aim of using mouse cells and drosophila cells was to possibly detect conserved features in evolution and the aim of using mouse cells in different cell behaviours was to analyze the contribution to differentiation as opposed to pluripotent cells.

Genome-Wide Replication Origins Maps

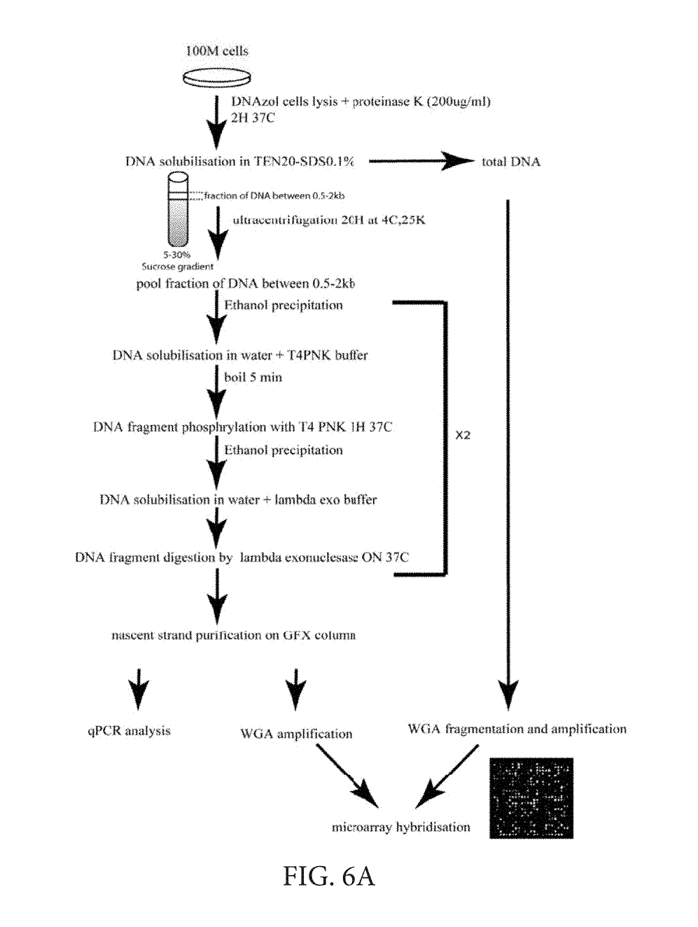

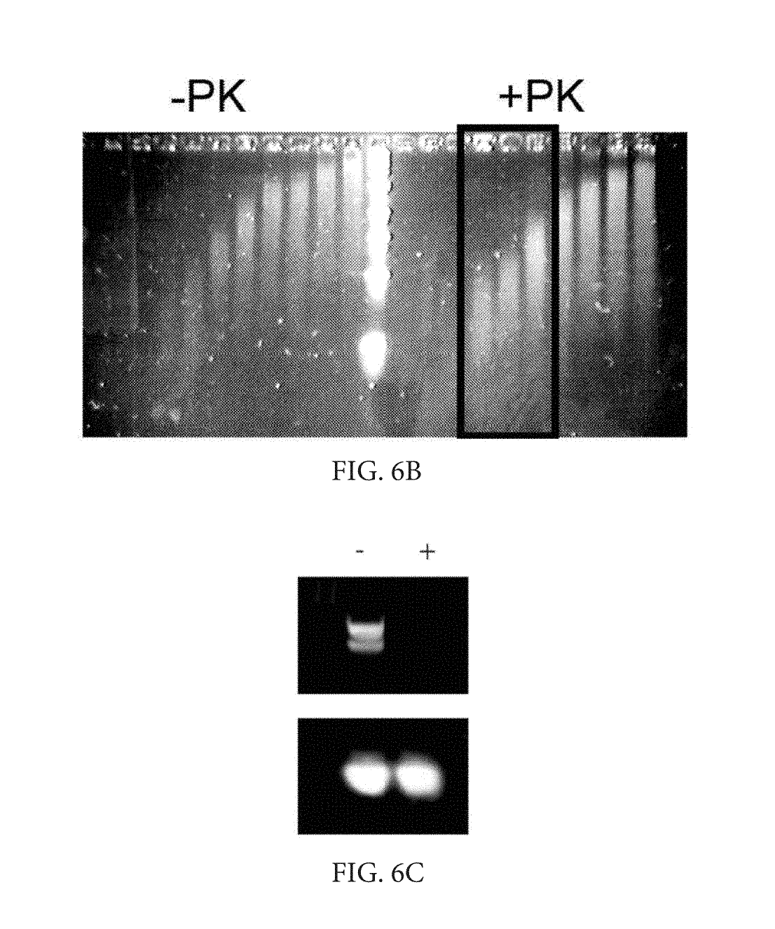

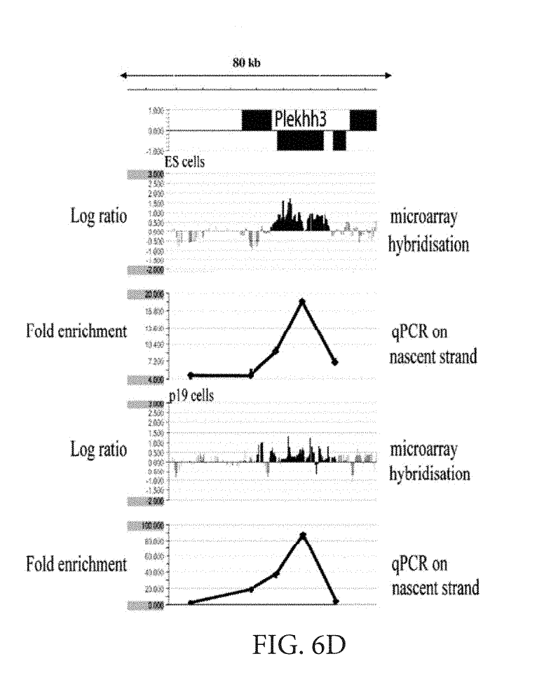

The RNA-primed nascent DNA procedure of preparation was initially improved using P 19 cells that grow in large amounts, and the method is detailed in Supplementary material and FIG. 6A-E. It was checked with up to 5 entirely different duplicates.

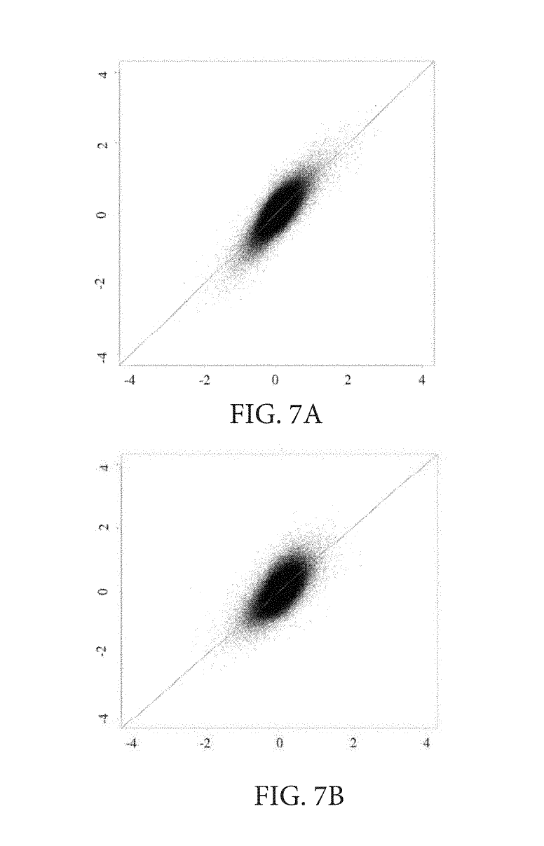

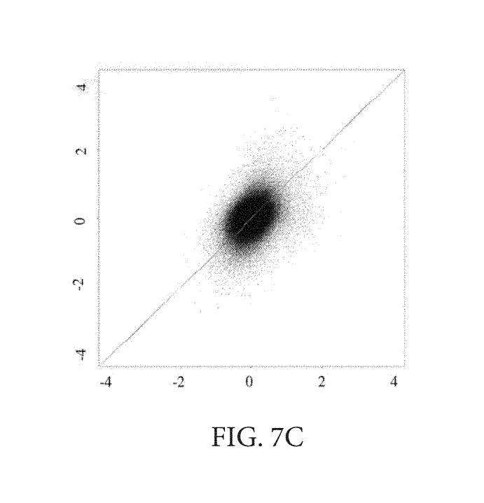

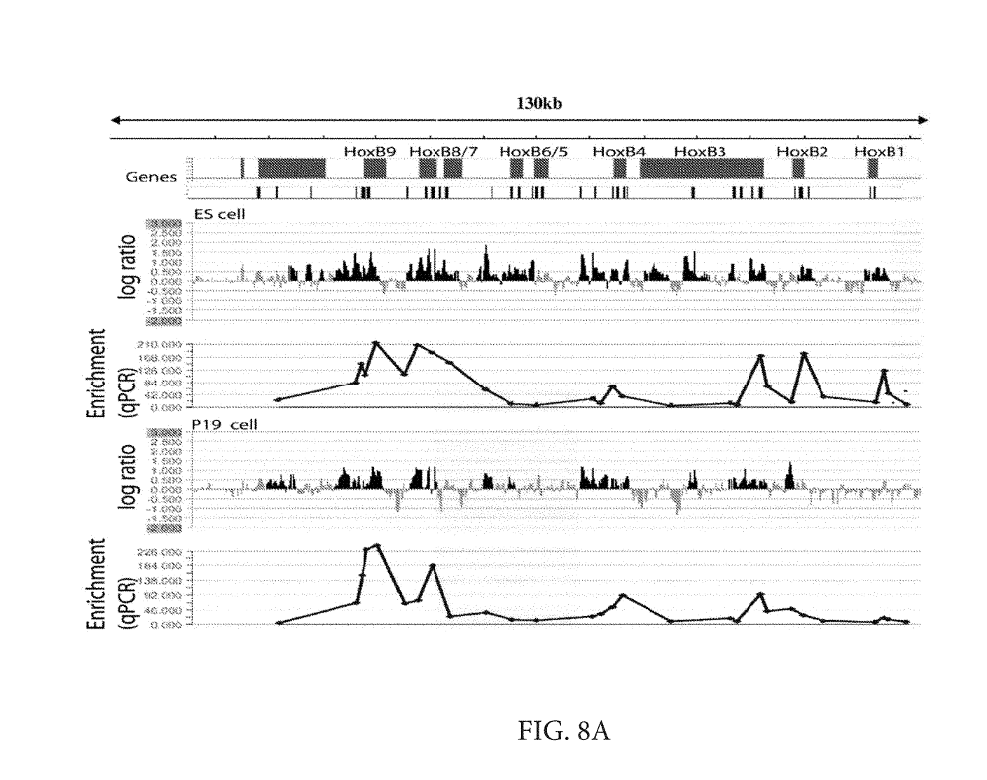

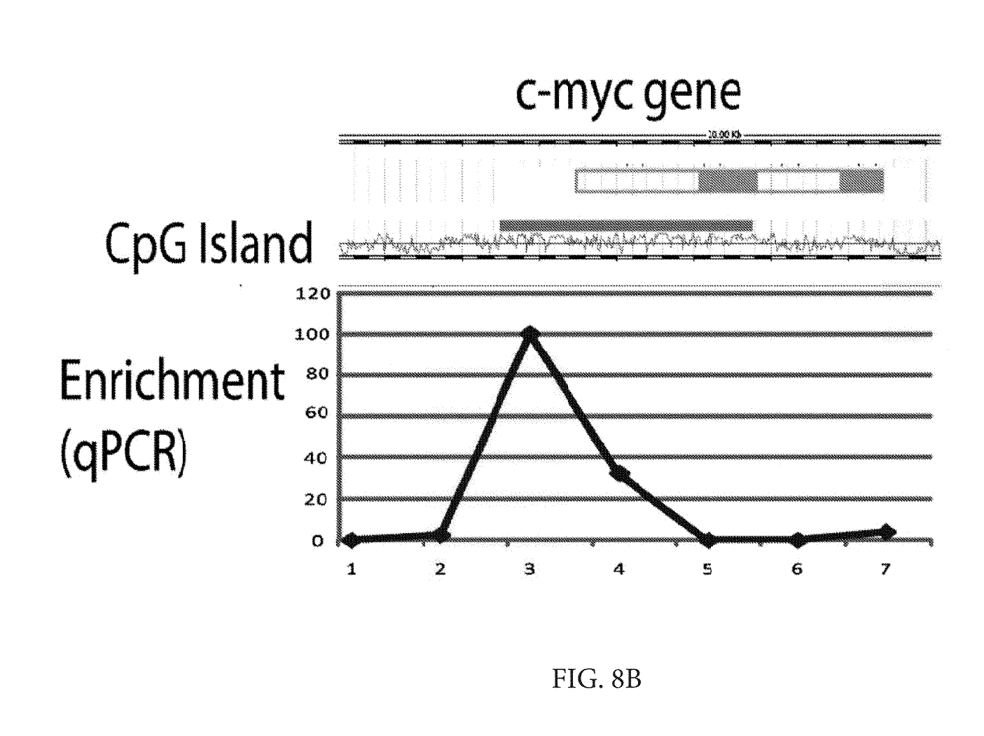

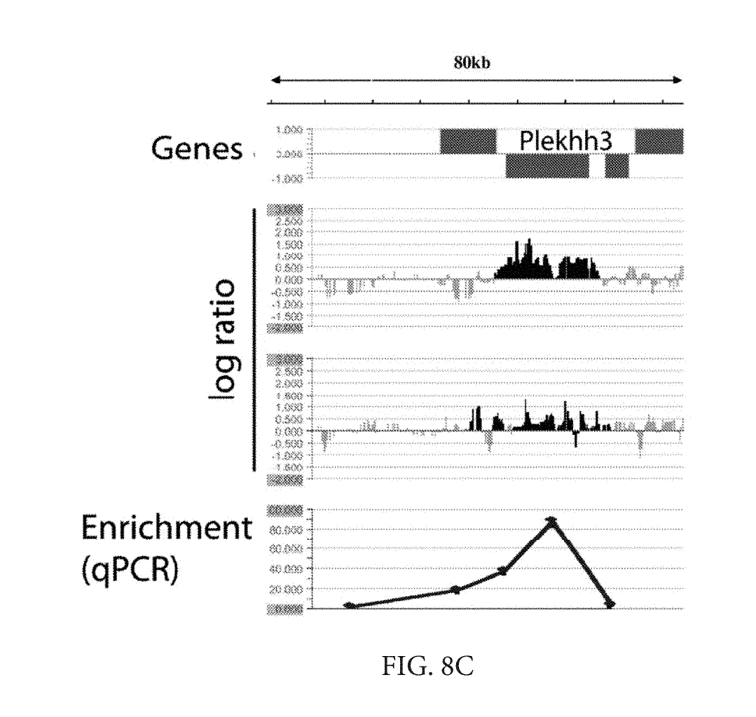

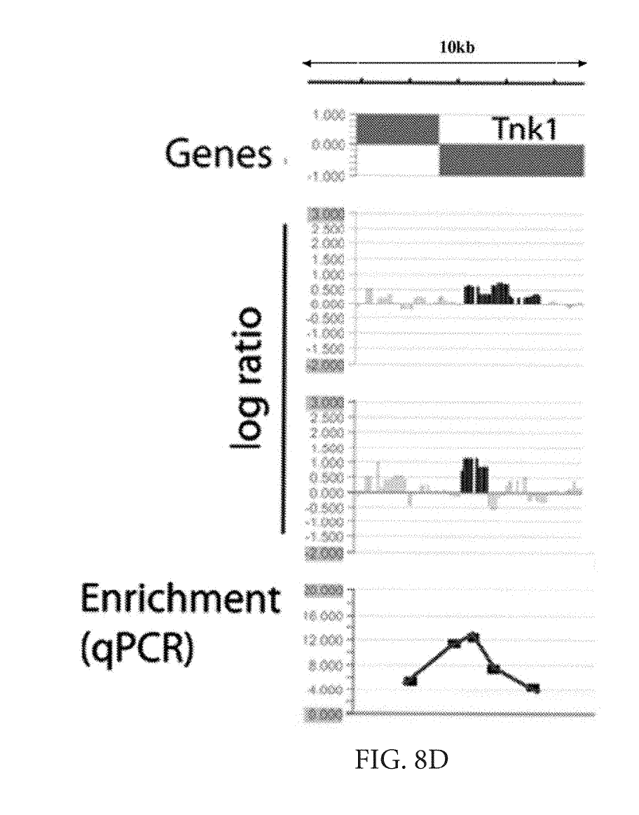

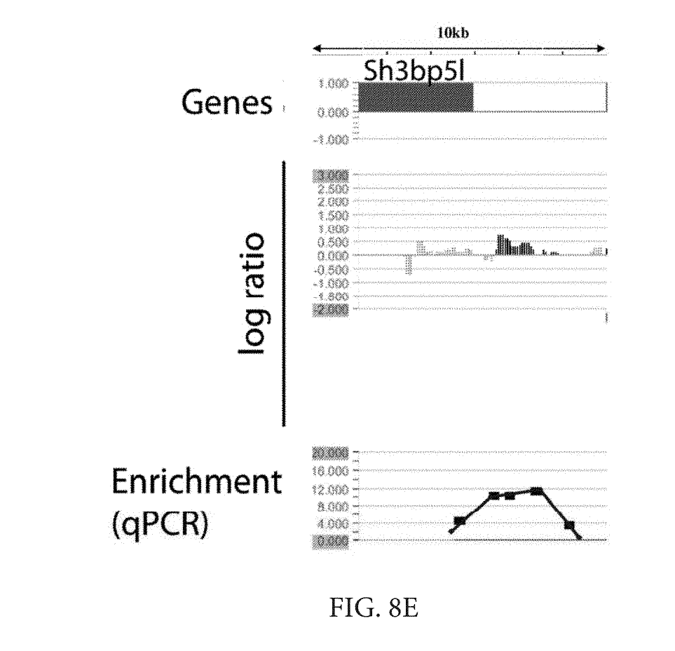

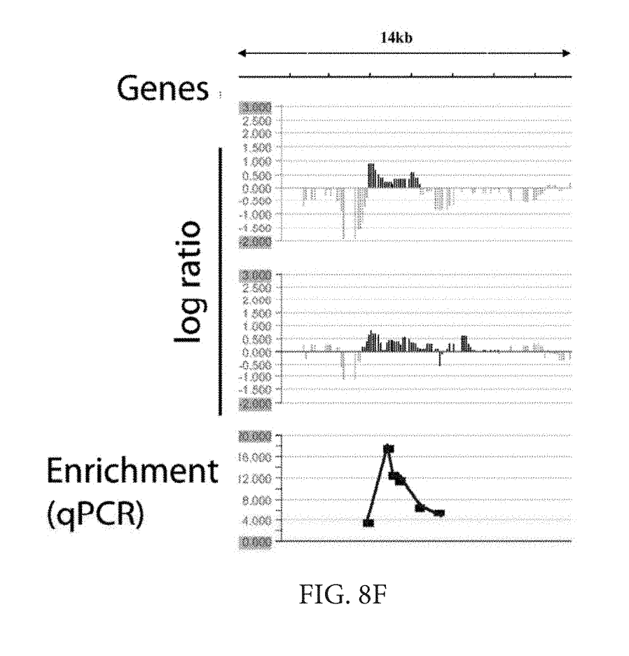

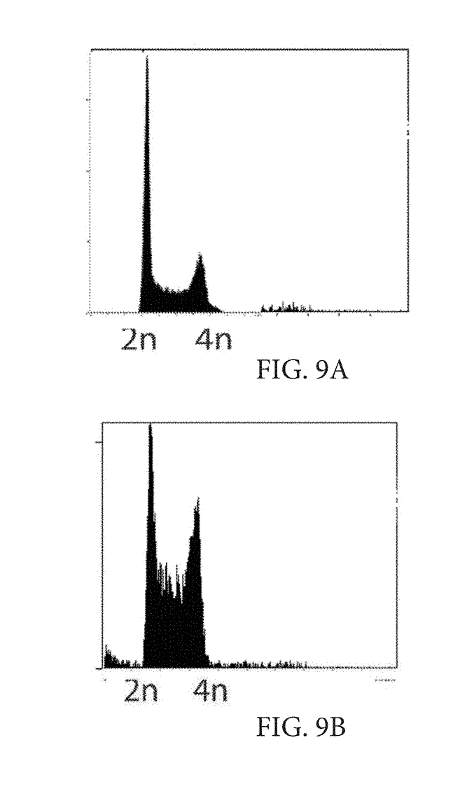

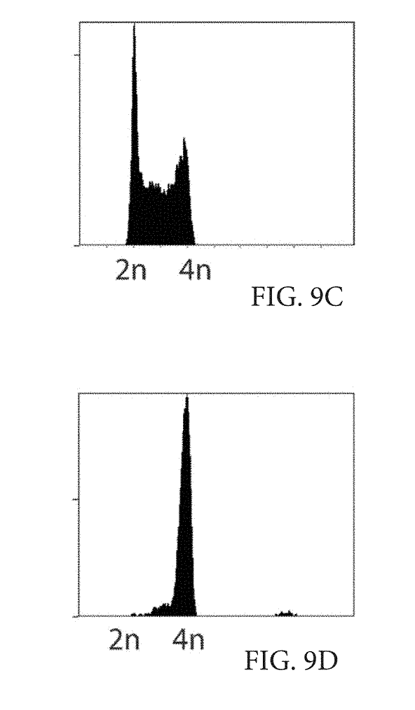

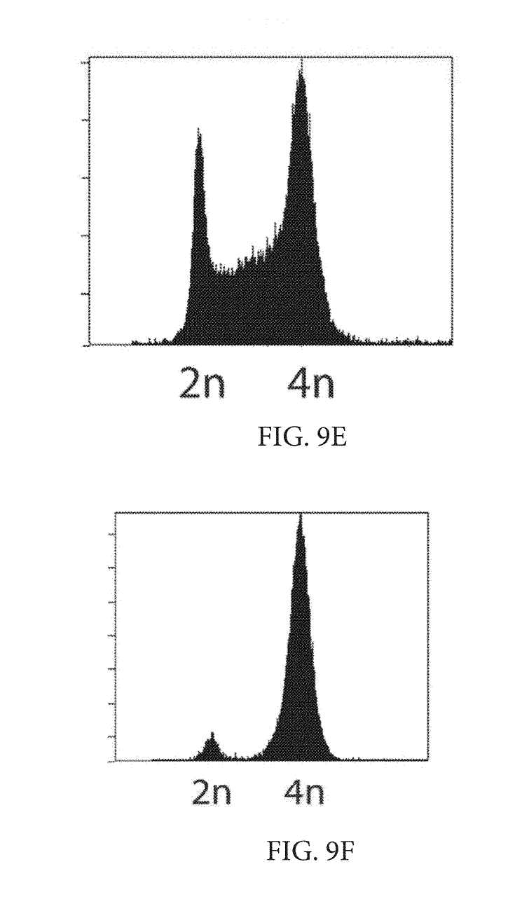

Nascent strand preparations were hybridized on tiling micro-array (Nimblegen, oligonucleotides spaced every 100 bp). The full data set consists of continuous 60.4 Mbp on mouse chromosome 11 and 118.3 Mbp of Drosophila genome. Origins maps show enrichment at specific genomic locations with a high degree of reproducibility (FIG. 1A-C and FIG. 7A-C). The Inventors validated the Ori maps of known origins by qPCR analysis of mouse e-Mye gene (FIG. 8B) and HoxB domain (FIG. 8A) as well as of randomly chosen putative Oris (FIG. 8C-F). No specific signals was observed when total DNA or ‘Nascent Strands’ from mitotic cells was used for hybridization (Fig IB, IC and FIG. 9), or when NS was RN Ase treated before exonuclease digestion (data not shown), confirming the specificity of our purification procedure. Importantly, no replication origin could be detected when using ‘nascent strands’ purified from non-cycling mitotic cells, confirming the specificity of our purification scheme.

Replication Origins Distribution

The method used allows scoring potentially all activated origins activated during the whole S-phase as exponentially growing cells were used. If there is existing variation between the origins activated in a given cell relative to another in the same growing cell population, all the potential replication initiation sites will be scored. In such conditions, the Inventors scored 146700 potential origins per genome, similar for the both mouse pluripotent cell types (FIG. 1 b, but MEF cells display less origins, 84800 potential origins per genome (Fig Ib, and this is associated with an increase in origin length. 60.2% MEF origins were also observed in the two pluripotent cell lines cell lines.

Replication origins of Drosophila cells display the same length than MEF cells but with density higher than mouse cells (see later).

With regard to genes, mouse replications origins were found to be significantly associated with genes (p<0.001; Fig ID). More particularly, origins overlap significantly (p<0.001) promoter and exonic sequences in all murine cell types (Fig IE). Drosophila origins were found associated significantly with exonic sequences (FIG. 1 G). Highly transcribed genes are enriched in replication origins, suggesting that transcription may facilitate origin specification and/or firing (Fig IH and 11).

Replication Origins are Determinated by CpG Island-Like Regions

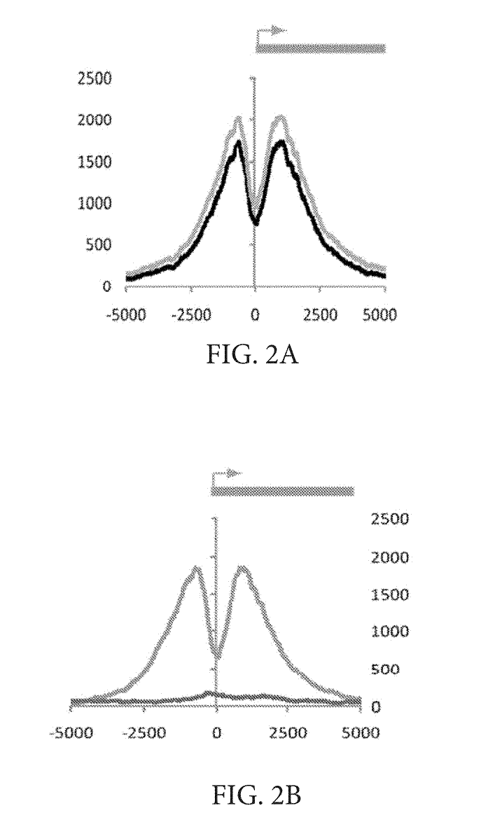

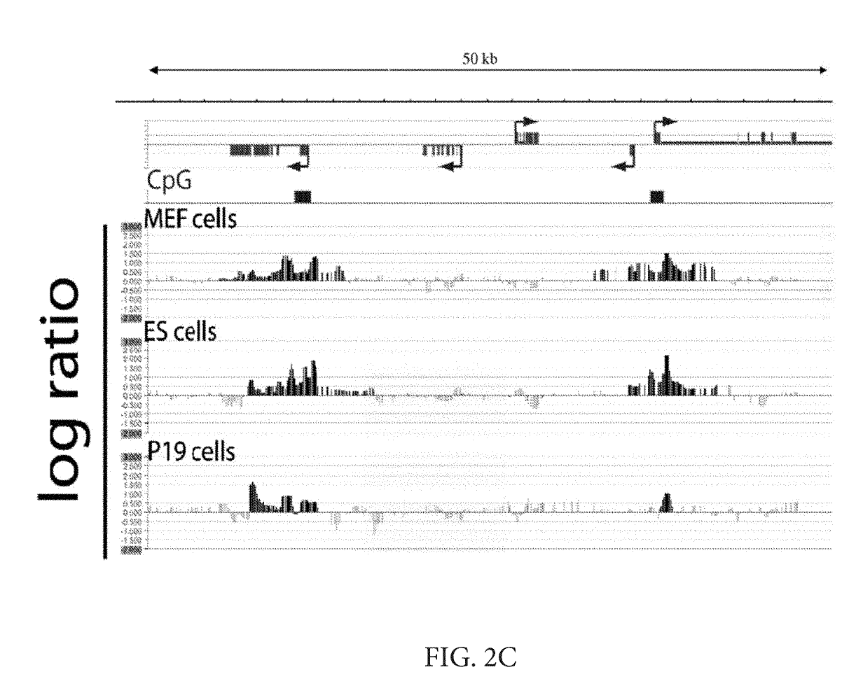

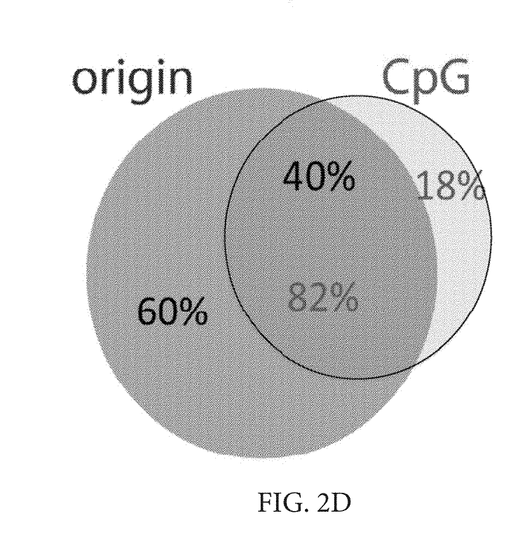

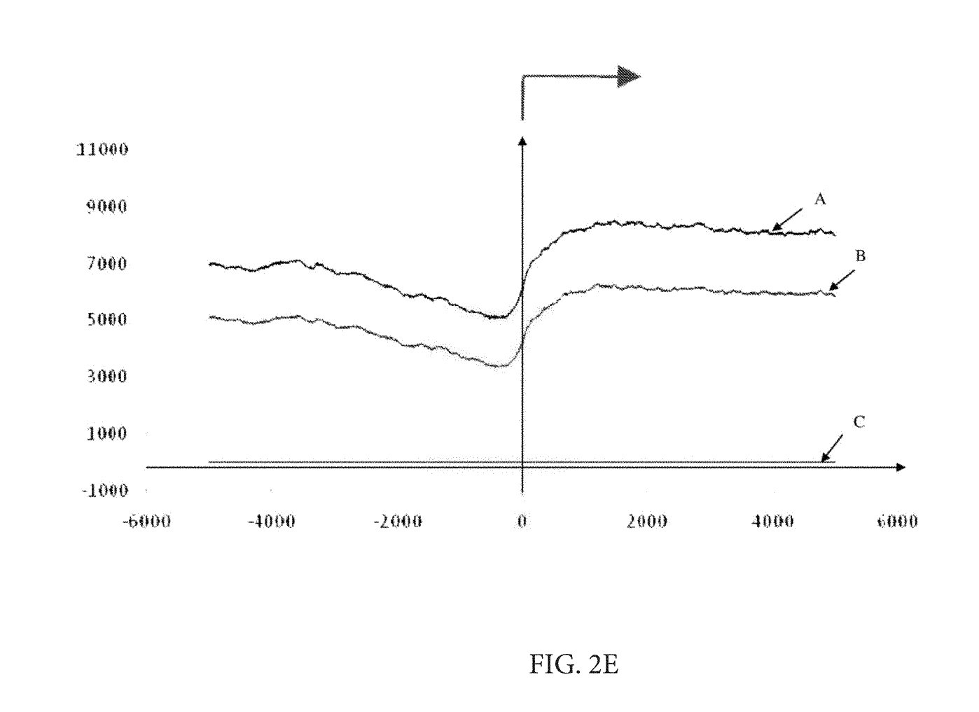

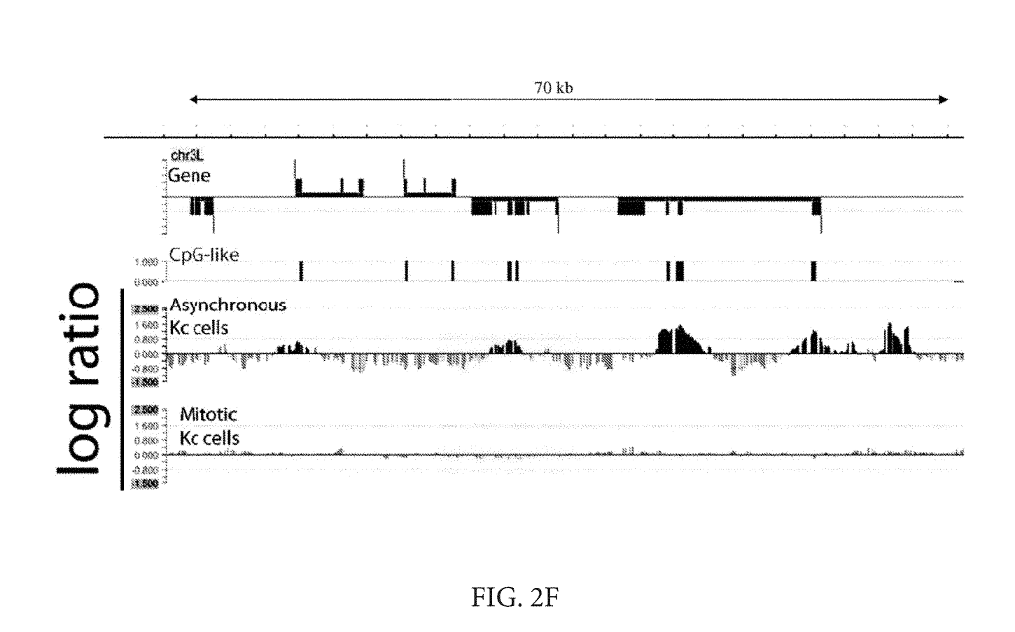

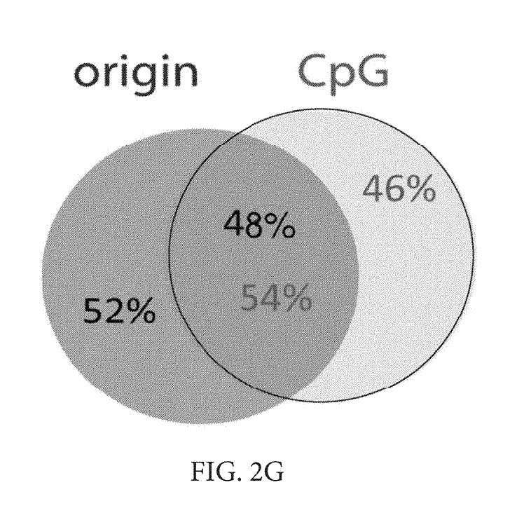

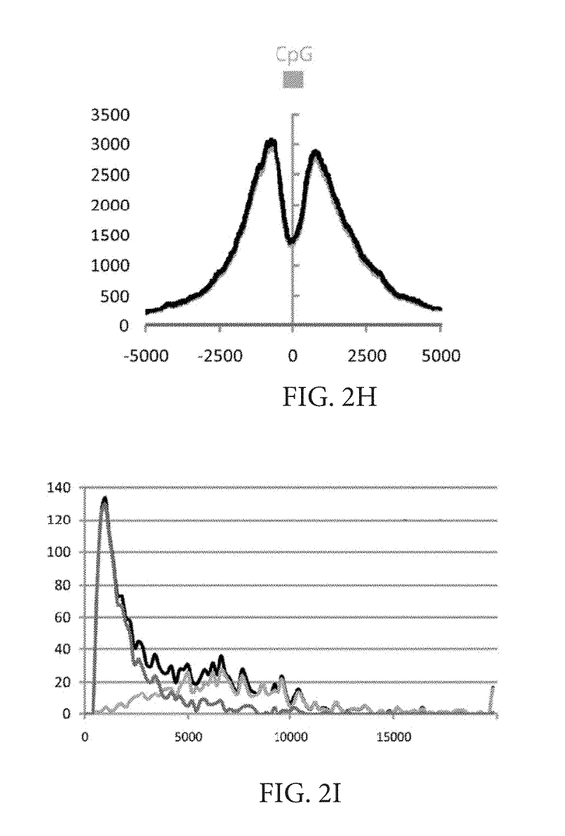

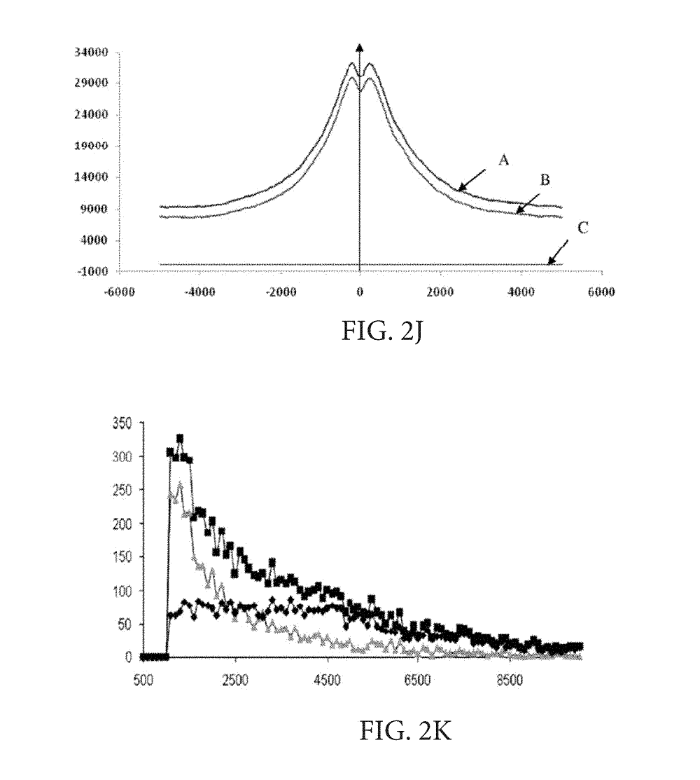

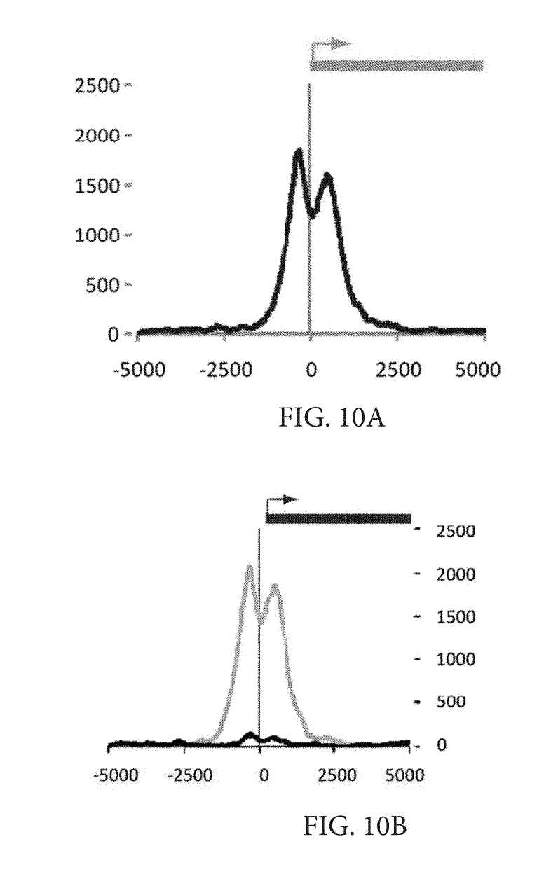

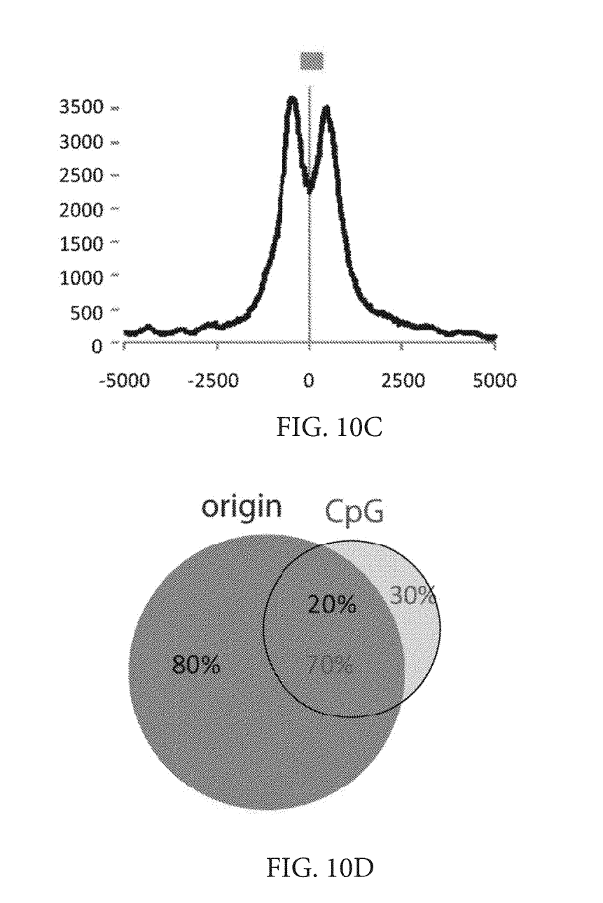

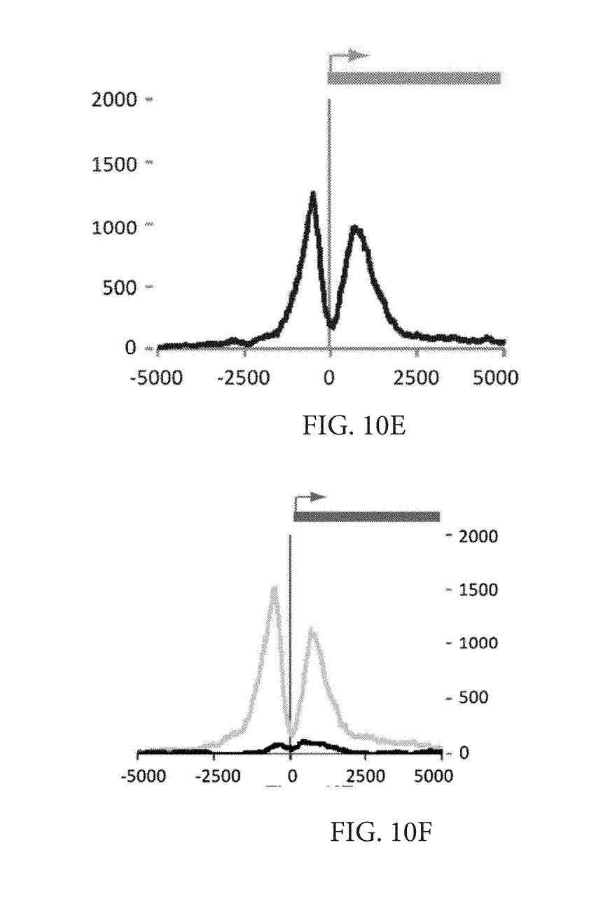

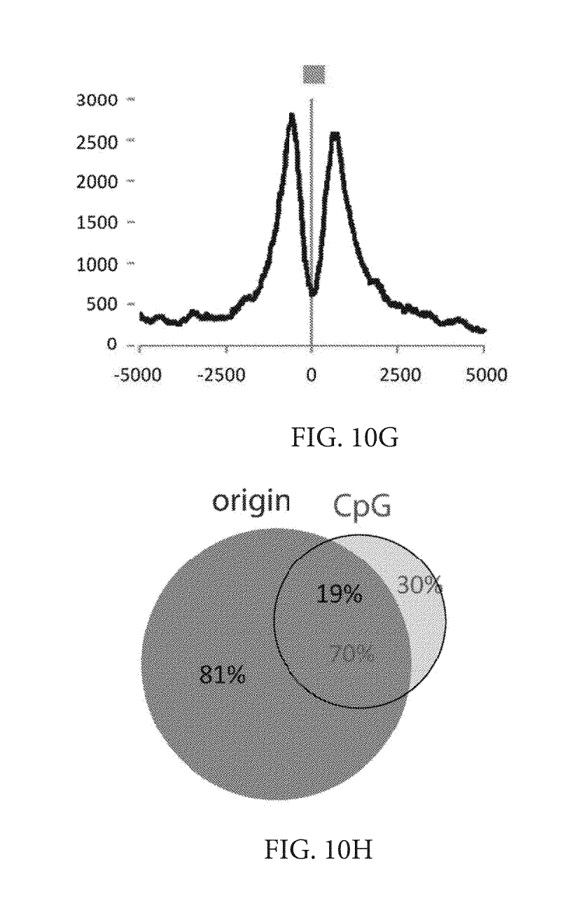

Given their association with transcriptional units and with promoter regions, the Inventors examined the distribution of replication origins around the transcription start sites (TSS) in mouse cells. Overall, TSS are highly associated with nascent strands signals (FIGS. 2A, 10A and 10E). Strikingly, the Inventors observed a strong bimodal distribution around the TSS, with a low probability to get nascent strands overlapping the TSS, whereas the two borders were enriched. This suggests that, at these DNA replication origins, two nascent strands initiation sites are used, bordering the TSS. A possible explanation was that a genetic element at the TSS was not itself used as a DNA synthesis site but driving initiation on its borders. In Drosophila cells, TSS are not enriched in origins, in contrast to mouse cells (FIG. 2E). In agreement, the Inventors did not observe the mouse bimodal distribution but detected an increase of origin density within gene as opposed to the promoter region (FIG. 2E).

Mammalian promoters and particularly from highly expressed genes are CpG-rich while genes highly regulated during development are often CpG-poor or free. CpG-rich sequences are known as CpG Islands (CGI). To better understand the bimodal distribution, the Inventors divided our analysis on TSS CpG-positive (n=820) and TSS CpG-free (n=434) separately. Notably, nascent strands specific signals are strongly associated with CGI-positive promoter while CG I-negative promoters are devoid of such signals in all three mouse cell lines (FIGS. 2B, 10B and 10F). The Inventors next looked at origins distribution around CGI. The Inventors found that replication origins were strongly associated with CGI in all mouse cell lines (FIGS. 2C, 2D, 10D and 10H). Moreover, origins distribution was also found to be bimodal around CGI (FIGS. 2H, 10C and 10G).

CGI are usually defined as regions of 200 pb min in length with 60% of CG-richness and a ratio of CpG observed/CpG>0.6. Because cytosine methylation is almost inexistent in drosophila melanogaster, there is not a genome-wide bias toward eliminating CpG dinucleotides during evolution. The drosophila genome nevertheless contains region with identical properties as mammalian CGI. The Inventors delimitated these regions as CGI-like sequences. More of the half of CGI-like regions (54%) in drosophila cells and more than 70% of these sequences in mouse cells lines are associated with replication origin. These values drop to 32% and 43% for the randomized origins dataset. Moreover, the population of origins that is longer than average is even more associated with this sequence (82% in mice, FIGS. 2I and 2K). Altogether, the strong association of replication origins with CpG Island positive and highly transcribed genes may suggest that active genes are occupied by components from the pre-replicative complex.

The Inventors concluded that sequences related to CGI are determinant for localization of origins in mice as well as drosophila, regardless of their genomic position, e. g. not only in promoter region, consistent with presence of CGI-like sequences in exonic region from drosophila genome. These results also provided a novel possible function for CGI sequences conserved both in vertebrates and invertebrate species.

Nevertheless, CpG island rich sequences do not recognize the majority of replication origins (see FIGS. 2D, 10D and 10H). The Inventors conclude that replication origins might be specified by additional mechanisms, and the primary sequence was one possibility.

The Majority of Metazoan Replication Origin Shares a Common Motif

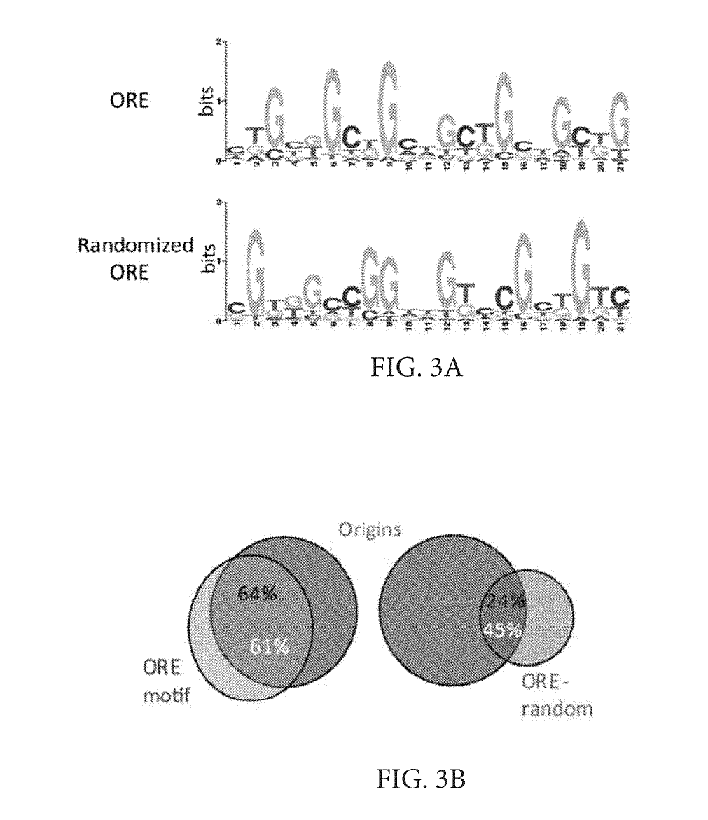

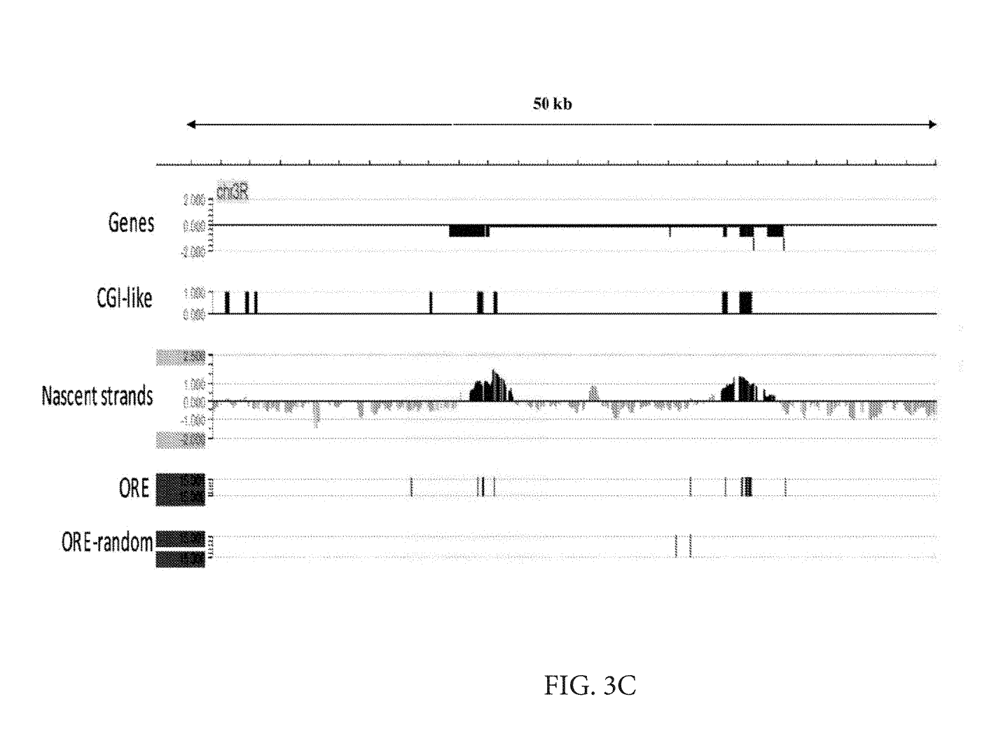

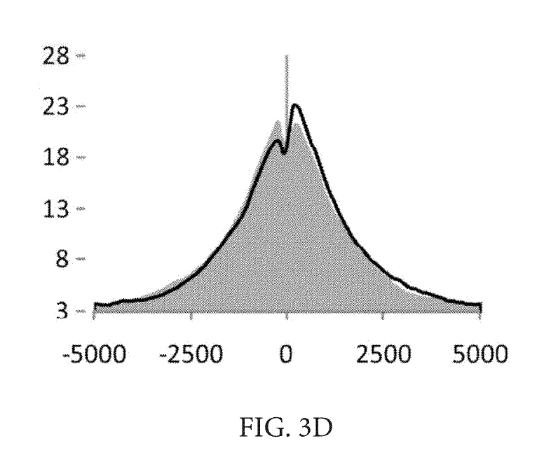

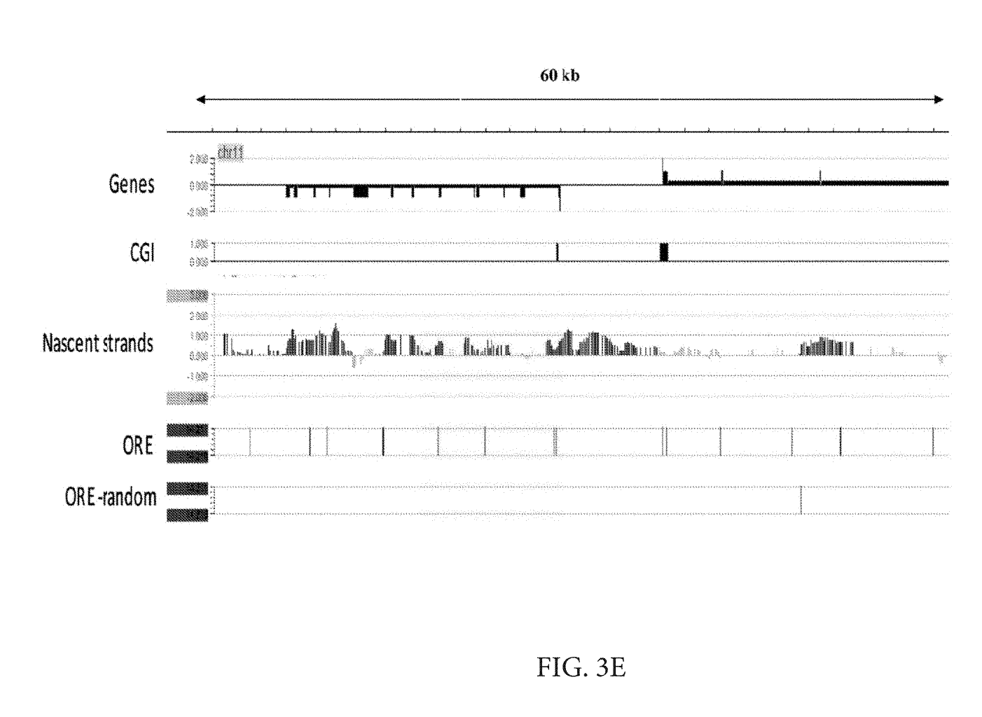

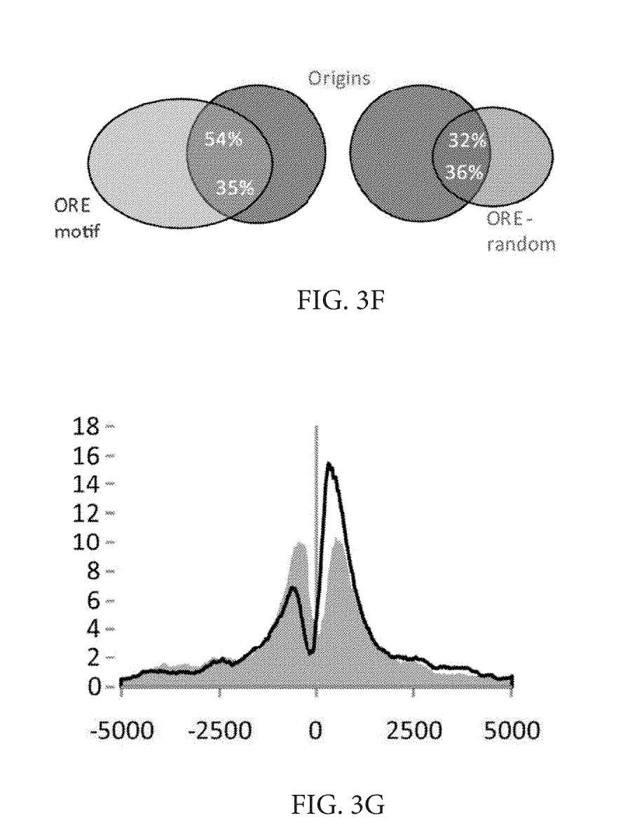

No consensus sequence is known to be associated with metazoan origins. Nevertheless, the Inventors hypothesized that such a sequence could potentially be identified in drosophila origins because of the compactness of the fly genome. As a first approach, fifteen 3 kb length origins sequence were submitted to the MEME server (http://meme.sdsc.edu/meme4_4_O/intro.html) using default settings. A repetitive G-rich motif was found. When matched on the drosophila genome, this motif detected a large (>50%) proportion of replication origins. Several rounds of optimization gave rise to a repeated G-rich sequence that contained G every three nucleotides along the repeat (FIG. 3A). Because of its unique ability to detect Oris (see below) and of its repetitive nature, this motif was dubbed OGRE for Origin G-rich Repeated Element. When the Inventors looked for the occurrence of this motif genome-wide (using FIMO server; http://meme.sdsc.edu/meme/fimo-intro.html), the Inventors found that it had good predictive value as it was associated to more than two thirds of the origins (FIG. 3B). In contrast, changing the nucleotide position in the motif results in poor origins prediction, indicating that the primary sequence, and not only GC-content, was essential (FIGS. 3B and 3C). Interestingly, the repeat number influenced Ori prediction: increasing the number of repeats in OGRE significantly improved prediction, whereas decreasing the repeat number lowered it. Cumulative origins signals associated with the motif again revealed a bimodal distribution, similarly to CGI-like domain, but the motif detects more origins than these domains (FIG. 3D). Moreover, the Inventors observed that NS signal associated with OGRE was orientated upstream of the initiation site itself (IS). The Inventors further observed that the motif found in drosophila cells was efficient for detecting the majority of replication origins mapped in MEF, ES and P19 cells (FIGS. 3E and 3F). Permuting the motif position again strongly reduced origin coverage by the motif, confirming that the primary sequence of the motif was important. Nascent Strands signals around OGRE showed an asymmetric bimodal distribution, like in drosophila cells (FIG. 3G). Finally the Inventors found that OGRE was present in the majority of the previously characterized Oris. The OGRE has a significant predictive power. Indeed, also two thirds of all OGRE occurrences mapped very close to replication origins in drosophila cells. In mammals, the OGRE is also predictive, much better than the 3.3% predictive value of the ACS element in budding yeast. Altogether, these results suggest that metazoan replication origins display a conserved element which might be involved either for origin specification and/or origin activation/firing.

Hieratical Organization of Replication Origins in Metazoan

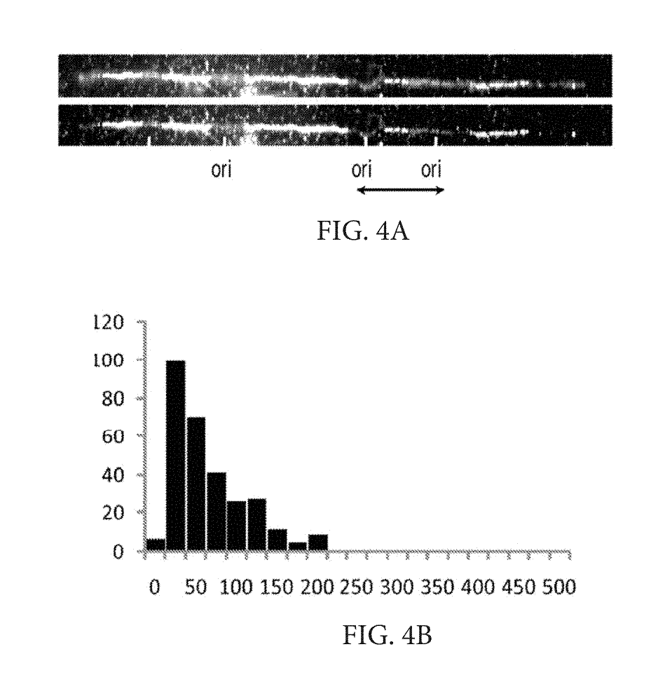

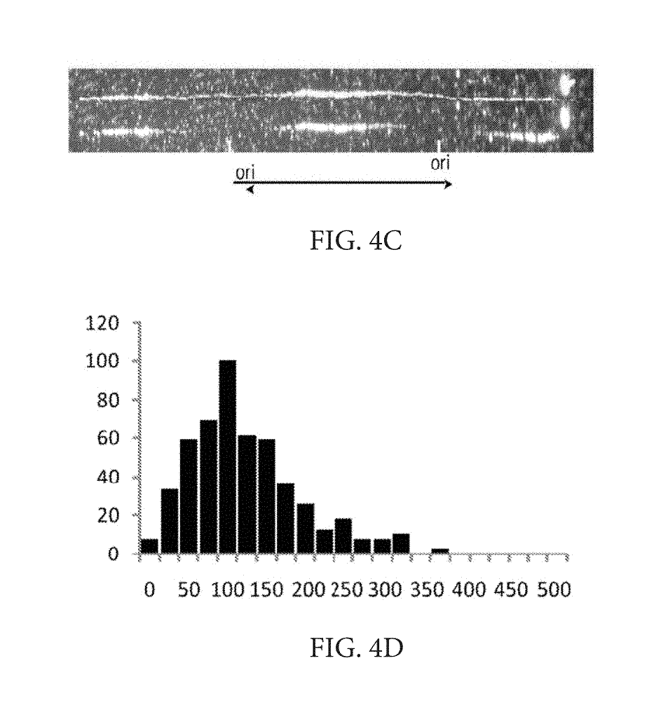

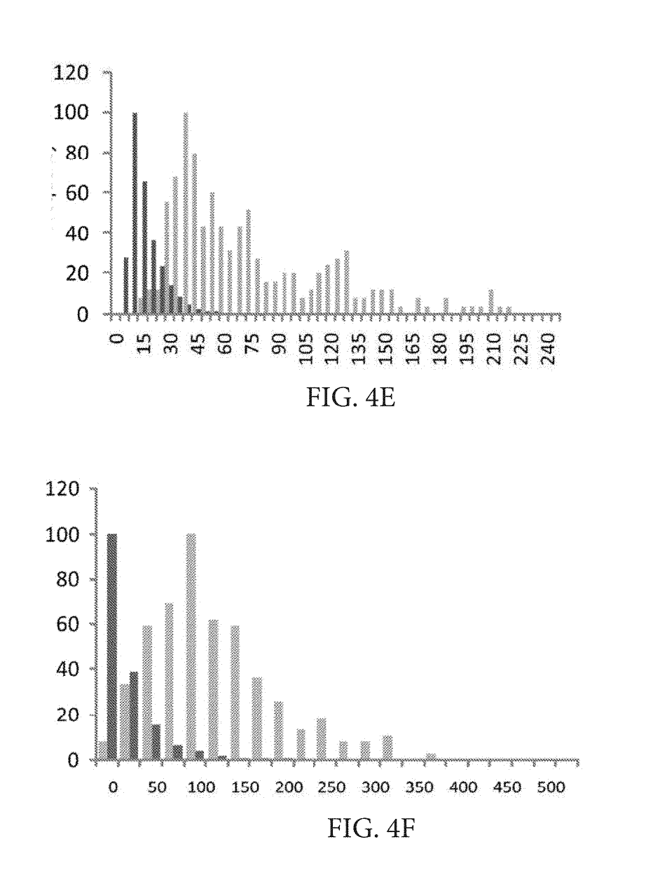

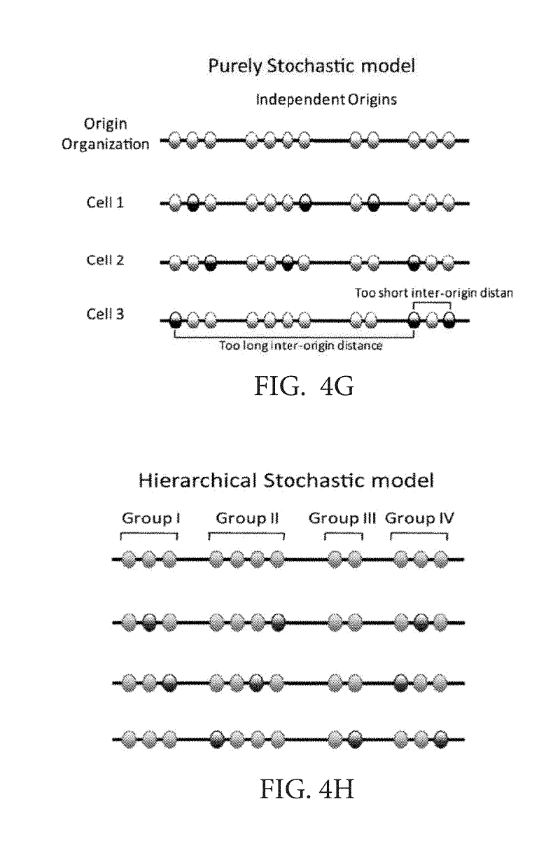

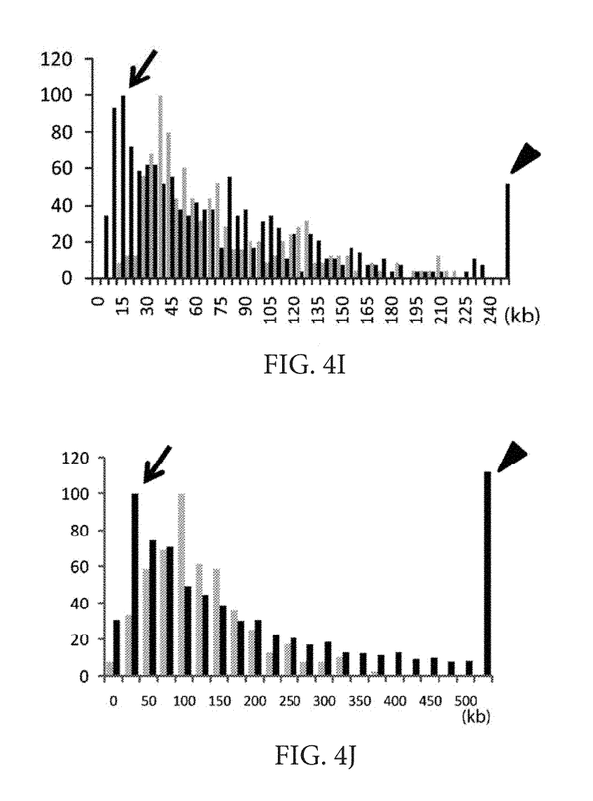

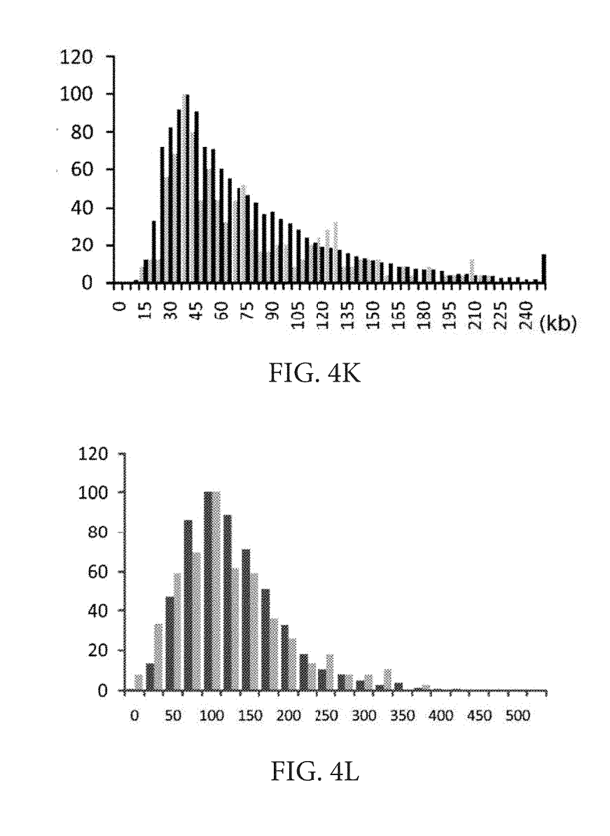

Genome-wide data permit to identify sites which can serve as DNA replication origins, but do not permit to have a view of origin usage along individual DNA molecules. Analysis at the single molecule level can be performed by DNA combing, where replicating DNA is labeled with pulses of modified nucleotide in vivo, and high molecular weight then stretched at a constant rate onto a slide. This method allows the precise determination of replication speed and inter-origin distances (FIG. 4). MEFs, ES and P 19 cells replicate their DNA with a similar fork speed of 1.5 kb/min, similar to rates observed in human cells. Drosophila cells exhibit a nearly two-fold slower fork (0.8 kb/min).

Sequential dual nucleotide labeling to determinate fork direction and bi-directional origins of replication was performed. The Inventors observed a near two-fold difference in inter-origin distances between mouse cells (139 kb) and drosophila cells (73 kb) (FIG. 4A-D). The smaller inter-origin distance in Kc cells might be a consequence of the more compact drosophila genome. Of note, pluripotent or differentiated mouse cells have similar inter-origins spacing even though they differ in cell cycle profile and origin repertoire.

If all mapped origins were activated (firing efficiency of 100%) the resulting very short inter-origin distance distribution would be significantly different from the distribution observed by DNA combing (FIGS. 4E and 4F). As exemplified by MEF cells, the origin density of all potential origins was 4.3 fold higher than the density observed by DNA combing; indicating that 1 in every 4.3 origins on average is fired in individual DNA molecules (firing efficiency of ˜23%). Our results both in Drosophila and mouse cells are consistent with the findings that metazoan origins, like yeast Oris [Heichinger C, et al. EMBO J 2006 Nov. 1; 25(21):5171-9], are redundant and that only a small proportion of them is effectively used at each cell cycle. The Inventors next wanted to model genome-wide origin usage in MEF cells to recapitulate the origins firing pattern observed in single cell. The Inventors first tested the possibility that origins were fired purely stochastically (FIG. 4G). Using a firing efficiency of 23%, the mean inter-origin distances of randomly fired Oris was identical to the value obtained by DNA combing. However, the simulated inter-origin distance distribution was significantly different from the distribution obtained in combing experiments (FIG. 4J). The purely stochastic distribution was characterized by populations of short and long inter-origin distances not observed in combing experiments (arrows in FIG. 4H). The group of large inter-origin distances was in agreement with the random gap problem, with the consequence that too large replicons could not completely replicate and that a large number of gaps of unreplicated DNA would persist at the end of S phase [Hyrien O, et al. Bioessays. 2003 February, 25(2):116-25; Laskey R A. J Embryol Exp Morphol. 1985 November,•89 Suppl:285-96]. In the second model that the Inventors called the hierarchical stochastic model, groups of adjacent origins are functionally linked together into domains over a defined distance that defines the replicon, where activation of one origin silences the others (FIG. 4H). Origins were thus grouped, considering their distribution along the genome, and one single Ori per domain was allowed to fire randomly. Strikingly, the simulated inter-origin distances were significantly similar to the combing data (FIG. 4L). Importantly, the hierarchical stochastic model was also functional in ES and in Kc cells (FIG. 4I, 4K and data not shown). This model requires optimization of the clustering parameters (the average size of the cluster). Nevertheless, the model is thus robust and can accommodate changes in origin density and firing efficiency. Overall, these data suggest that DNA replication origins are in large excess in metazoans and have a flexible use. Metazoan replicons appear constituted of groups of potential and flexible adjacent origins where activation of one origin suppresses the surrounding ones.

Density of Replication Origins in Chromosome 11

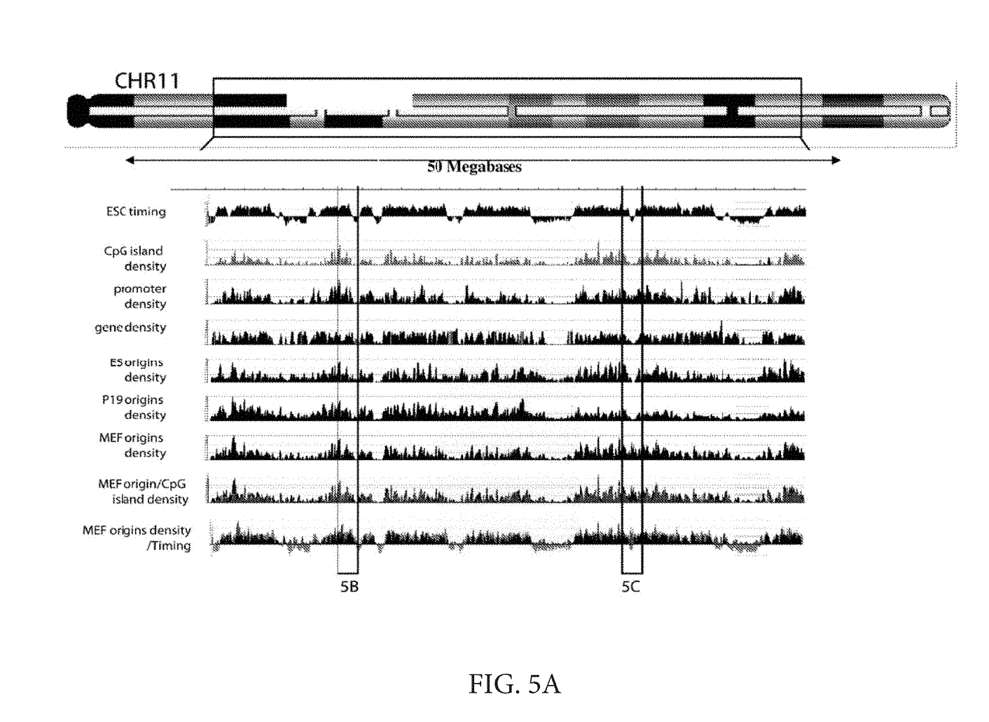

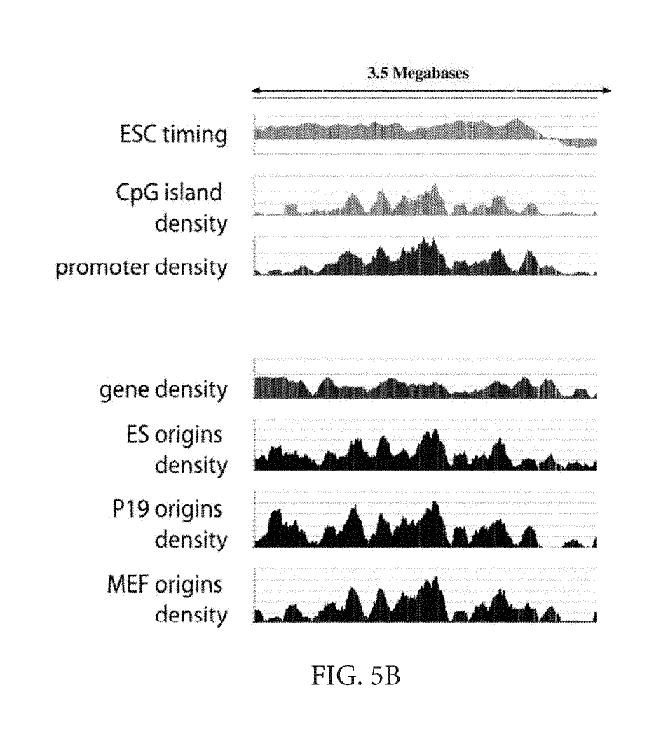

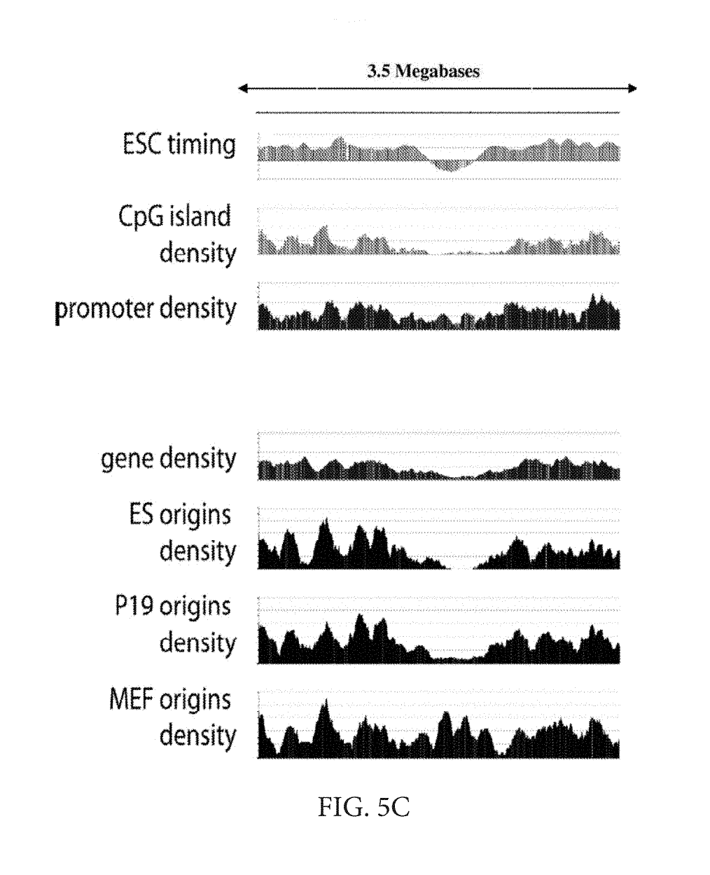

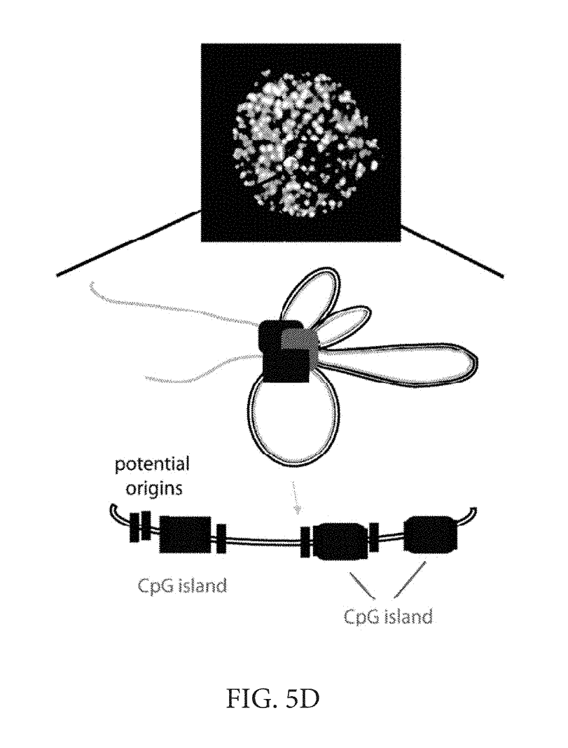

DNA replication origins are often synchronously activated in clusters. The Inventors looked at the origin density on areas of 70 Kb in mice and 50 Kb in Drosophila through a sliding window every 10 bp. First, the Inventors observed that zones of high density of origins were at similar positions along chromosome 11, for all three mouse cells lines (Fig SA). Then, the Inventors compared these areas with other genomic features such as density in genes, promoter and CpG islands. For example, the areas of density origins coincide well with areas of density of CpG islands in MEF cells (Fig SA). A similar trend was observed for ES and P19 cells (data not shown). The replication timing of different ESC cells was recently published, and showed a very high conservation profile between distantly related pluripotent cells. The Inventors observed a strong correlation between early replicated regions and areas of high origins density in ES and P19 cells (FIG. 5A). In MEFs, the Inventors also observed a strong correlation between early replicated regions and areas of high origins density (FIG. 5A). For example, a 3.5 Mb early replicating domain is enriched in replication origins in all mouse cell lines tested (FIG. 5B). This region is also rich in CpG Island, promoter and genes. In a late replicating part of chromosome 11, pluripotent cells display low density replication origins (Fig SC). However, MEP show strong origin activity, suggesting that this region could replicate early in this cell type. Similar, but albeit weaker, trends where observed for drosophila replication origins (data not shown).

The inventors thus propose that a replication cluster includes consecutive groups of adjacent flexible Oris, each set constituting a replicon, that are activated synchronously (see FIG. 4H). The selection of a given Ori within each replicon might be achieved through several mechanisms. Selection itself might depend on the cell fate or the organization of the chromatin domain. The Ori interference mechanism has been described in yeast [Brewer and Fangman, Science. 1993 Dec. 10; 262(5140):1728-31; Lebofsky R, et al. Mol Biol Cell. 2006 December; 17(12):5337-45] where firing at one Ori inhibits close-by Oris and this phenomenon could lead to the 100-120 Kbp average size of the replicon. Alternatively, control elements or chromosome organization might control firing in the cluster. For example, activation of one Ori might promote looping out of the replicon and silencing of the other potential Oris. The CpG Islands seems to be a putative control element for origin organization (FIG. 5D).

Materials and Methods

Cell Culture

The HEK-293 cell line stably expressing EBNA1 (HEK293 EBNA1+) that was cultivated in Dulbecco's modified Eagle's minimal medium containing 10% fetal calf serum and 220 μg/ml Neomycin.

Plasmid Replication Assay.

2 μg of the reporter plasmid containing the various origin variants were transfected into HEK293 EBNA1+ cell line using Lipofectamine2000 (Life technologies) according to manufacturers instructions. Transfections with comparable efficiencies were verified by visualizing GFP-positive cells. Six days post-transfection, cells were harvested according to the HIRT protocol (Hirt B. (1967) J Mol Biol. 26, 365-9). After washing with PBS, cells were first equilibrated in 5 ml TEN buffer (10 mM Tris-HCl pH 7.5, 1 mM EDTA, 150 mM NaCl). After resolubilization in 1.5 ml TEN and equal volume of 2×HIRT buffer (1.2% SDS, 20 mM Tris-HCl pH 7.5, 20 mM EDTA) was then added for cell lysis. The lysate was then incubated at 4° C. for 16 h, in the presence of 1.25 M NaCl. After centrifugation for 1 h at 20000×g at 4° C., DNA was purified by phenol-chloroform extraction and digested with 40 U DpnI (NEB) in presence of RNase (Roche). Digested DNA (300 ng) was electroporated into Electromax DH10B competent cells (Invitrogen) and ampicillin-resistant colonies, representing the number of recovered plasmids, were counted. The wild-type oriP-plasmid was always transfected in parallel and the number of resulting colonies was used for normalization.

The deletion was tested using a specific restriction enzyme MsII recognizing a motif found in close proximity to the G quadruplex.

Cell and Tissue Culture

hESC cells were maintained in an appropriated medium under 5% CO, at 37° C. CD34(+)ve hematopoietic progenitor cells were isolated from human cord blood using previously established protocols. These cells were treated with erythropoietin for 3 or 6 days (day3, day6). HMEC cells were generated as previously described (ref). HMEC cells were initially immortalized using a stably transfected shRNA against p53 gene (ImM-1). A subclones of ImM-1 cell line was later generated by stably transfecting human RAS (ImM-2) or WNT (ImM-3).

Nascent Strand Isolation

Nascent strands were purified and sequenced as previously described with the following modifications:

SNS-Seq Analysis

Illumina reads (50 bp, single-end) from each SNS-seq replicates were trimmed and aligned to hg38 using bowtie (as previously described Cayrou et al). Peaks were called using a combination of two peak calling programs, MACS2 and SICER. Peaks were called using MACS2 (default parameters plus --bw 500 -p le-5 -s 60 -m 10 30 --gsize 2.7e9), followed by peak calling by SICER (parameters: redundancy threshold=1, window size (bp)=200, fragment size=150 effective genome fraction=0.85, gap size (bp)=600, FDR=1e-3). MACS2 peaks that intersect SICER peaks from each sample were merged using bedtools intersect to generate a comprehensive list of all human DNA initiation sites (IS). Blacklisted regions as defined by ENCODE project (hg38, ENCSR636HFF) were subtracted from our final human DNA replication origins list, leaving 320,748 regions. Summits of origins were defined by calculating the highest number of SNS-seq reads in bins of 50 bp from 25 bp sliding windows. Middle point of the bin with highest number of reads was considered the summit of the IS.

Quantification and Classification of DNA Replication Origins

Regions that correspond to Quantification of DNA replication origins were done using the R-package DiffBind (TMM_minus_background), using all human/mouse origin coordinates. Following TMM normalization, we calculated average normalized SNS-seq counts across 19 samples for each origin and assigned each origin to a quantile (Q1-Q10) accordingly. Each quantile consisted of 32,074 origins. Super origins were defined as having >50 normalized SNS-seq counts in 18 or more samples. Tissue specific origins were determined by selecting origins that had >50 average normalised SNS-seq counts in the tissue of interest, which was more than 2 standard deviations further from the average normalised SNS-seq counts in other untransformed cell types.

Data Analysis

Heatmaps, boxplots and other plots were generated using ggplot2 and heatmap.2. Both Pearson's and Spearman's correlation matrices were calculated in R using (command cor( )). Comparison between genomic coordinates (quantiles, alternative origin mapping methods, histone/TF/ORC binding sites) as well as generation of randomized genomic coordinates were computed using bedtools suite (intersectBed with a minimum overlap of 1 bp, bedtools shuffle -chrom). COVERAGE (G4)—add. Principal component analysis (PCA) was carried out in R. SNS-seq read density plots and heatmaps were generated using deeptools (plotProfile, plotHeatmap). Where necessary, genome coordinates were converted between different genome assemblies using UCSC LiftOver (UCSC Toolkit).

Analysis of Base Composition in Genomic Regions

Base composition analysis was done using HOMER (REF), with 100 bp as window size taking IS summit as the peak center. The data was then visualized using Microsoft excel.

Evolutionary Conservation Analysis

Refseq exons, introns and promoter regions (−500 to 0 bp upstream of transcription start sites) and Phastcon scores (Phastcon20way) were downloaded from UCSC browser (last update December 2017). Mean cumulative phastcon scores of each set of regions were calculated using R and bedtools suite (bedtools coverage). Human origin coordinates were converted to mouse coordinates either using LiftOver (UCSC toolkit).

Prediction of DNA Replication Origins in the Human Genome

Human and mouse genome was divided into paired 500 bp windows (Watson and crick strands separately) with a sliding window size of 100 bp using bedtools (makewindows) suite. We then calculated the number of each nucleotide (A,C,G,T) in each paired window (bedtools nuc). Paired 500 bp windows were evaluated with permissive (min 0.25% G in the first window, followed by another 500 bp window in which G content drops by 8-40%, with a max A/T content 0.21 or strict (min 0.28% G in the first window, followed by another 500 bp window in which G content drops by 8-40%. In addition, we only retained window pairs, if and only if the total G content +/−the center of the window pair was >25%) criteria. The window pairs that were retained were then merged using bedtools merge to identify non-overlapping putative origin regions.

1. A method for initiating the replication of a first double stranded deoxyribonucleic acid (DNA) molecule in a pluricellular eukaryotic cell, said first molecule being devoid of self-replication capabilities in a pluricellular eukaryotic cell, said method comprising:
inserting into said first double stranded DNA molecule at least one multicellular DNA replication origin to obtain a second double stranded DNA molecule, said replication origin consisting essentially of a sequence from about 50 to about 800 nucleotides, said replication origin comprising at least a regulatory element (RE), and an initiation site (IS), wherein said IS is located downstream to the RE at about 50 to 800 nucleotides, the RE comprising at least a nine-nucleotide sequence consisting of at least three uninterrupted origin repeating elements (OGRE), each OGRE having one of the following sequences:
N1N2G, wherein N1 is a G or a A and N2 is a pyrimidine or a A;
N3GN4, wherein N3 is T or G and N4 is G or C; and
GN5N6, wherein N5 is different from N6, N5 is a G or a C and N6 is a T or a A;
introducing said second double stranded DNA molecule into a pluricellular eukaryotic cell; and then
identifying the replicated molecules and measuring their efficiency of replication,
wherein the replication of said second DNA molecule comprising said DNA replication origin is solely initiated, in a pluricellular eukaryotic cell, by the presence of said replication origin.

2. The method according to claim 1, wherein the step of consists of identifying the nascent DNA synthesized from the IS of the inserted DNA replication origin in said a second double stranded DNA molecule, the nascent DNA identifying said IS.

3. The method according to claim 1, wherein said RE forms a potential G quadruplex structure.

4. The method according to claim 1, wherein the replication origin comprises one of the sequences as set forth in SEQ ID NO: 34 to SEQ ID NO: 78.

5. The method according to claim 1, wherein said RE interacts with a preRC complex.

6. The method according to claim 1, wherein said RE controls progression of a replication loop initiated in said IS.

7. A process for preparing a recombinant non-naturally occurring double stranded DNA multicellular eukaryotic replicative vector, or replicative vector, comprising at least one multicellular DNA replication origin as the unique means for replicating the vector in a pluricellular eukaryotic cell or cell extract, said process comprising a step of
inserting into a first recombinant non-naturally occurring double stranded DNA vector at least one DNA molecule comprising at least one multicellular DNA replication origin in order to obtain a multicellular eukaryotic replicative vector, said replication origin consisting essentially of a sequence from about 50 to about 800 nucleotides, said replication origin comprising at least a regulatory element (RE), and an initiation site (IS), wherein said IS is located downstream to the RE at about 50 to 800 nucleotides, the RE comprising at least a nine-nucleotide sequence consisting of at least three uninterrupted origin repeating elements (OGRE), each OGRE having one of the following sequences:
N1N2G, wherein N1 is a G or a A and N2 is a pyrimidine or a A;
N3GN4, wherein N3 is T or G and N4 is G or C; and
GN5N6, wherein N5 is different from N6, N5 is a G or a C and N6 is a T or a A;
introducing said replicative vector into a pluricellular eukaryotic cell; and then
recovering the vectors resulting from the replication;
wherein said a first recombinant non-naturally occurring double stranded DNA vector being devoid of self-replicative capabilities in a pluricellular eukaryotic cell, and
wherein the inserted at least one multicellular DNA replication origin allows said DNA vector to self-replicate in a pluricellular eukaryotic cell or cell extract.

8. The method according to claim 7, wherein the at least one multicellular DNA replication origin comprises at least one of the sequences as set forth in SEQ ID NO: 34 to SEQ ID NO: 78.

9. The method according to claim 7, wherein said RE forms a potential G quadruplex structure.

10. The method according to claim 7, wherein said RE interacts with a preRC complex.

11. A double stranded DNA vector comprising as its unique replicative DNA replication origin a replication origin consisting essentially of a sequence from about 50 to about 800 nucleotides, said replication origin comprising at least a regulatory element (RE), and an initiation site (IS), wherein said IS is located downstream to the RE at about 50 to 800 nucleotides, the RE comprising at least a nine-nucleotide sequence consisting of at least three uninterrupted origin repeating elements (OGRE), each OGRE having one of the following sequences:
N1N2G, wherein N1 is a G or a A and N2 is a pyrimidine or a A;
N3GN4, wherein N3 is T or G and N4 is G or C; and
GN5N6, wherein N5 is different from N6, N5 is a G or a C and N6 is a T or a A;
wherein said vector is devoid of bacterial or unicellular eukaryotic replication origin.

12. The vector according to claim 11, wherein the at least one multicellular DNA replication origin comprises at least one of the sequences as set forth in SEQ ID NO: 34 to SEQ ID NO: 78.

13. The vector according to claim 11, said RE forms a potential G quadruplex structure.

14. The vector according to claim 11, wherein said RE interacts with the preRC complex.


SEQUENCE LISTING (78 sequences)

SEQ ID NO: 1 - DNA, 7 bp, Artificial sequence
  eukaryote replication origin
  misc_feature (1)..(2): n is a, c, g, or t
  misc_feature (4)..(4): N= G or A
  misc_feature (5)..(5): N= T or C
  misc_feature (6)..(7): n is a, c, g, or t
nngnnnn

SEQ ID NO: 2 - DNA, 7 bp, Artificial sequence
  eukaryote replication origin
  misc_feature (1)..(2): n is a, c, g, or t
  misc_feature (3)..(3): N= T or G
  misc_feature (5)..(5): N= G or C
  misc_feature (6)..(7): n is a, c, g, or t
nnngnnn

SEQ ID NO: 3 - DNA, 7 bp, Artificial sequence
  eukaryote replication origin
  misc_feature (1)..(2): n is a, c, g, or t
  misc_feature (3)..(3): N= G or C
  misc_feature (4)..(4): N= T or A
  misc_feature (6)..(7): n is a, c, g, or t
nnnngnn

SEQ ID NO: 4 - DNA, 21 bp, Artificial sequence
  eukaryote replication origin
shgcygsyggmgcygshgstg

SEQ ID NO: 5 - DNA, 21 bp, Artificial sequence
  eukaryote replication origin
ckgykgckgcdgckgcdgykg

SEQ ID NO: 6 - DNA, 13 bp, Artificial sequence
  eukaryote replication origin
gtcccagtcccag

SEQ ID NO: 7 - DNA, 13 bp, Artificial sequence
  eukaryote replication origin
tgctgctgctgct

SEQ ID NO: 8 - DNA, 13 bp, Artificial sequence
  eukaryote replication origin
tatatatatatat

SEQ ID NO: 9 - DNA, 13 bp, Artificial sequence
  eukaryote replication origin
agcagcagcagca

SEQ ID NO: 10 - DNA, 13 bp, Artificial sequence
  eukaryote replication origin
gttgctgctgctg

SEQ ID NO: 11 - DNA, 13 bp, Artificial sequence
  eukaryote replication origin
tcagacatcttag

SEQ ID NO: 12 - DNA, 13 bp, Artificial sequence
  eukaryote replication origin
agcagcagcaaca

SEQ ID NO: 13 - DNA, 13 bp, Artificial sequence
  eukaryote replication origin
cagacatcttagg

SEQ ID NO: 14 - DNA, 13 bp, Artificial sequence
  eukaryote replication origin
agacatcttaggc

SEQ ID NO: 15 - DNA, 13 bp, Artificial sequence
  eukaryote replication origin
cagcagcagcagc

SEQ ID NO: 16 - DNA, 13 bp, Artificial sequence
  eukaryote replication origin
taacgtgtggtga

SEQ ID NO: 17 - DNA, 13 bp, Artificial sequence
  eukaryote replication origin
tgttgctgctgct

SEQ ID NO: 18 - DNA, 13 bp, Artificial sequence
  eukaryote replication origin
cagcagcagcaac

SEQ ID NO: 19 - DNA, 9 bp, Artificial sequence
  eukaryote replication origin
tgctgctgc

SEQ ID NO: 20 - DNA, 9 bp, Artificial sequence
  eukaryote replication origin
cagcagcag

SEQ ID NO: 21 - DNA, 9 bp, Artificial sequence
  eukaryote replication origin
ctgctgctg

SEQ ID NO: 22 - DNA, 12 bp, Artificial sequence
  eukaryote replication origin
ctctctctctct

SEQ ID NO: 23 - DNA, 12 bp, Artificial sequence
  eukaryote replication origin
tctctctctctc

SEQ ID NO: 24 - DNA, 13 bp, Artificial sequence
  eukaryote replication origin
agctggggcggca

SEQ ID NO: 25 - DNA, 13 bp, Artificial sequence
  eukaryote replication origin
cagctggggcggc

SEQ ID NO: 26 - DNA, 13 bp, Artificial sequence
  eukaryote replication origin
gctggggcggcag

SEQ ID NO: 27 - DNA, 13 bp, Artificial sequence
  eukaryote replication origin
agcagctggacac

SEQ ID NO: 28 - DNA, 13 bp, Artificial sequence
  eukaryote replication origin
cagcagctggaca

SEQ ID NO: 29 - DNA, 13 bp, Artificial sequence
  eukaryote replication origin
gcagcagctggac

SEQ ID NO: 30 - DNA, 13 bp, Artificial sequence
  eukaryote replication origin
cagctggacacac

SEQ ID NO: 31 - DNA, 13 bp, Artificial sequence
  eukaryote replication origin
agcagactgggcg

SEQ ID NO: 32 - DNA, 21 bp, Artificial Sequence
  ori from from mouse cc4 gene
gggggcggggagggaaggggg

SEQ ID NO: 33 - DNA, 27 bp, Artificial Sequence
  origin from mouse cc2 gene
gggggatggggttggaatgggggcggg

SEQ ID NO: 34 - DNA, 32 bp, Artificial sequence
  eukaryote replication origin
tgggagtgggtgggtaggggagcagggcgggt

SEQ ID NO: 35 - DNA, 33 bp, Artificial sequence
  eukaryote replication origin
cggggaggggacgaaggggagagaggatgggga

SEQ ID NO: 36 - DNA, 31 bp, Artificial sequence
  eukaryote replication origin
tggtgggggtgggggtaggggtggggggaga

SEQ ID NO: 37 - DNA, 33 bp, Artificial sequence
  eukaryote replication origin
agggggagtgggtgggtaggggattgggggggt

SEQ ID NO: 38 - DNA, 36 bp, Artificial sequence
  eukaryote replication origin
aggagggggatgtggaggaggagggggagggggggt

SEQ ID NO: 39 - DNA, 32 bp, Artificial sequence
  eukaryote replication origin
tgggctgggggctggagggggggatactggga

SEQ ID NO: 40 - DNA, 22 bp, Artificial sequence
  eukaryote replication origin
aggggacaggggcaggggtggt

SEQ ID NO: 41 - DNA, 26 bp, Artificial sequence
  eukaryote replication origin
agggggaggagagagggggtagggga

SEQ ID NO: 42 - DNA, 28 bp, Artificial sequence
  eukaryote replication origin
tgggaagggtttttttgggggggcggga

SEQ ID NO: 43 - DNA, 23 bp, Artificial sequence
  eukaryote replication origin
tgactttgggggtggggtggggt

SEQ ID NO: 44 - DNA, 24 bp, Artificial sequence
  eukaryote replication origin
agggttgggggagggggatggaga

SEQ ID NO: 45 - DNA, 26 bp, Artificial sequence
  eukaryote replication origin
tggtggggagggtggtggggtgggga

SEQ ID NO: 46 - DNA, 26 bp, Artificial sequence
  eukaryote replication origin
agggggatgggagggggaatgggggc

SEQ ID NO: 47 - DNA, 51 bp, Artificial sequence
  eukaryote replication origin
agggggagtgggagtgtaggggatcgggggggggggtgtgagtatggggga

SEQ ID NO: 48 - DNA, 27 bp, Artificial sequence
  eukaryote replication origin
agggagagggaggatgggatagggggt

SEQ ID NO: 49 - DNA, 27 bp, Artificial sequence
  eukaryote replication origin
tggtaggggtggggtgggggctggggc

SEQ ID NO: 50 - DNA, 26 bp, Artificial sequence
  eukaryote replication origin
tggggatggggttgggggtaggggga

SEQ ID NO: 51 - DNA, 35 bp, Artificial sequence
  eukaryote replication origin
agggcaggcgggggtgggggggtgggggtgggggc

SEQ ID NO: 52 - DNA, 30 bp, Artificial sequence
  eukaryote replication origin
agggtgggtgggtgggtgggtgggtggggc

SEQ ID NO: 53 - DNA, 31 bp, Artificial sequence
  eukaryote replication origin
tggcggggcggggggggggtgcgggggaggt

SEQ ID NO: 54 - DNA, 24 bp, Artificial sequence
  eukaryote replication origin
aggagggagggagggagggaggga

SEQ ID NO: 55 - DNA, 23 bp, Artificial sequence
  eukaryote replication origin
tgggggtgggtgggtatggggga

SEQ ID NO: 56 - DNA, 33 bp, Artificial sequence
  eukaryote replication origin
cgggggggatggggggtcctcagggggcaggga

SEQ ID NO: 57 - DNA, 29 bp, Artificial sequence
  eukaryote replication origin
cgctgtggggtggggtggggtgggggaga

SEQ ID NO: 58 - DNA, 24 bp, Artificial sequence
  eukaryote replication origin
tggcatgggggtccgggggagggt

SEQ ID NO: 59 - DNA, 25 bp, Artificial Sequence
  human replication origin
  misc_feature (3)..(3): x = Gor C
  misc_feature (5)..(5): x = Gor C
  misc_feature (9)..(9): x = Gor C
  misc_feature (11)..(11): x = Gor C
  misc_feature (14)..(14): x = Gor C
  misc_feature (20)..(20): x = Gor C
  misc_feature (23)..(23): x = Gor C
  misc_feature (25)..(25): x = Gor C
  misc_feature (25)..(25): n = Gor C
ggngngggngnggngggggnggngn

SEQ ID NO: 60 - DNA, 30 bp, Artificial Sequence
  human replication origin
  misc_feature (3)..(3): n = Gor C
  misc_feature (4)..(4): n = Gor C
  misc_feature (9)..(9): n = Gor C
  misc_feature (10)..(10): n = Gor C
  misc_feature (15)..(15): n = A, Gor C
  misc_feature (21)..(21): n = Gor C
  misc_feature (28)..(28): n = A or G
  misc_feature (30)..(30): n = G or C
ggnnggggnnggggngggggnggggggngn

SEQ ID NO: 61 - DNA, 28 bp, Artificial Sequence
  human replication origin
  misc_feature (4)..(4): n = G or T
  misc_feature (10)..(10): n = G or C
  misc_feature (17)..(17): n = G or A
  misc_feature (28)..(28): n = G or C
gggngggggnggggggnggggggggggn

SEQ ID NO: 62 - DNA, 33 bp, Artificial Sequence
  human replication origin
  misc_feature (3)..(3): n = Aor G
  misc_feature (10)..(10): n = Aor C
  misc_feature (21)..(21): n = G or C
  misc_feature (22)..(22): n = G or C
  misc_feature (28)..(28): n = G or T
ggnggggggnggggcgggggnnggggcngggcc

SEQ ID NO: 63 - DNA, 23 bp, Artificial Sequence
  human replication origin
  misc_feature (4)..(4): n = G or C
  misc_feature (8)..(8): n = G or C
  misc_feature (10)..(10): n = G or C
  misc_feature (20)..(20): n = G or C
  misc_feature (21)..(21): n = G or C
gggngggngnggggcggggnncc

SEQ ID NO: 64 - DNA, 21 bp, Artificial Sequence
  human replication origin
  misc_feature (4)..(4): n = A or G
  misc_feature (7)..(7): n = C or G
  misc_feature (20)..(20): n = C or G
  misc_feature (21)..(21): n = C or G
gggnggnggcggggcggggnn

SEQ ID NO: 65 - DNA, 38 bp, Artificial Sequence
  human replication origin
  misc_feature (1)..(1): n = C or G
  misc_feature (3)..(3): n = C or G
  misc_feature (4)..(4): n = T or G
  misc_feature (10)..(10): n = C or G
  misc_feature (17)..(17): n = C or G
  misc_feature (23)..(23): n = T, C or G
  misc_feature (28)..(28): n =  C or G
  misc_feature (29)..(29): n =  C or G
  misc_feature (34)..(34): n =  C or G
ngnngggggnggggggngggggnggggnnggggngggg

SEQ ID NO: 66 - DNA, 23 bp, Artificial Sequence
  human replication origin
  misc_feature (3)..(3): n = C or G
  misc_feature (5)..(5): n = A or G
  misc_feature (11)..(11): n = C or G
  misc_feature (18)..(18): n = A or G
  misc_feature (23)..(23): n = C or G
ggngngggggnggggggnggggn

SEQ ID NO: 67 - DNA, 36 bp, Artificial Sequence
  human replication origin
  misc_feature (2)..(2): n = C or G
  misc_feature (3)..(3): n = T or G
  misc_feature (4)..(4): n = C or G
  misc_feature (9)..(9): n = C or G
  misc_feature (10)..(10): n = C or G
  misc_feature (16)..(16): n = C or G
  misc_feature (21)..(21): n = C or G
  misc_feature (28)..(28): n = A or G
  misc_feature (34)..(34): n = C or G
gnnnggggnngggggnggggnggggggngggggngg

SEQ ID NO: 68 - DNA, 16 bp, Artificial Sequence
  human replication origin
  misc_feature (11)..(11): n = A or G
gggggaggggngggcc

SEQ ID NO: 69 - DNA, 43 bp, Artificial Sequence
  mouse replication origin
  misc_feature (4)..(4): n = A or G
  misc_feature (8)..(8): n = A or G
  misc_feature (12)..(12): n = A or G
  misc_feature (16)..(16): n = A or G
  misc_feature (20)..(20): n = A or G
  misc_feature (24)..(24): n = A or G
  misc_feature (28)..(28): n = A or G
  misc_feature (32)..(32): n = A or G
  misc_feature (36)..(36): n = A or G
  misc_feature (40)..(40): n = A or G
gggngggngggngggngggngggngggngggngggngggnggg

SEQ ID NO: 70 - DNA, 28 bp, Artificial Sequence
  mouse replication origin
  misc_feature (12)..(12): n = A or G
  misc_feature (20)..(20): n = A or G
  misc_feature (28)..(28): n = A or G
gggggggggggngggggggngggggggn

SEQ ID NO: 71 - DNA, 31 bp, Artificial Sequence
  mouse replication origin
  misc_feature (6)..(6): n = A  or G
  misc_feature (8)..(8): n = A or G
  misc_feature (13)..(13): n = A or G
  misc_feature (18)..(18): n = A or G
  misc_feature (19)..(19): n = T or G
  misc_feature (26)..(26): n = A or G
  misc_feature (27)..(27): n = T or G
  misc_feature (28)..(28): n = A or G
  misc_feature (29)..(29): n = T or G
gggggngnggggnggggnnggggggnnnncc

SEQ ID NO: 72 - DNA, 34 bp, Artificial Sequence
  mouse replication origin
  misc_feature (2)..(2): n = A or G
  misc_feature (6)..(6): n = A or G
  misc_feature (8)..(8): n = A or G
  misc_feature (13)..(13): n = A or G
  misc_feature (18)..(18): n = A or G
  misc_feature (20)..(20): n = T, A or G
  misc_feature (24)..(24): n = A or G
  misc_feature (28)..(28): n = A or G
  misc_feature (32)..(32): n = A or G
gngggngnggganggggngngggngggngggngg

SEQ ID NO: 73 - DNA, 29 bp, Artificial Sequence
  mouse replication origin
  misc_feature (2)..(2): n = A or G
  misc_feature (6)..(6): n = A or G
  misc_feature (10)..(10): n = A or G
  misc_feature (14)..(14): n = A or G
  misc_feature (18)..(18): n = A or G
  misc_feature (21)..(21): n = A or G
  misc_feature (22)..(22): n = A or G
  misc_feature (26)..(26): n = A or G
gngggngggngggngggnggnngggnggg

SEQ ID NO: 74 - DNA, 27 bp, Artificial Sequence
  mouse replication origin
  misc_feature (1)..(1): n = A or G
  misc_feature (5)..(5): n = A or G
  misc_feature (17)..(17): n = A or G
  misc_feature (19)..(19): n = T or G
  misc_feature (25)..(25): n = A or G
ngggngggggggagggngngggggncc

SEQ ID NO: 75 - DNA, 25 bp, Artificial Sequence
  mouse replication origin
  misc_feature (4)..(4): n = A or G
  misc_feature (7)..(7): n = A or G
  misc_feature (8)..(8): n = A or G
  misc_feature (15)..(15): n = A or G
  misc_feature (20)..(20): n = A or G
gggnggnnggggggnggggngggcc

SEQ ID NO: 76 - DNA, 24 bp, Artificial Sequence
  mouse replication origin
  misc_feature (4)..(4): n = A or G
  misc_feature (18)..(18): n = T or G
  misc_feature (24)..(24): n = A or G
gggngggggggagggggngggggn

SEQ ID NO: 77 - DNA, 29 bp, Artificial Sequence
  mouse replication origin
  misc_feature (3)..(3): n = G or T
  misc_feature (7)..(7): n = G or T
  misc_feature (8)..(8): n = G or A
  misc_feature (13)..(13): n = G or A
  misc_feature (19)..(19): n = G or T
  misc_feature (26)..(26): n = G or A
  misc_feature (27)..(27): n = G or T
  misc_feature (28)..(28): n = G or A
  misc_feature (29)..(29): n = G or T
ggngggnnggggngggggnggggggnnnn

SEQ ID NO: 78 - DNA, 17 bp, Artificial Sequence
  mouse replication origin
  misc_feature (3)..(3): n = G or T
  misc_feature (8)..(8): n = G or T
  misc_feature (15)..(15): n = G or T
ggnggggnggggggngg